(19) United States
(12) Patent Application Publication
(10) Pub. No.: US 2010/0284915 A1
(43) Pub. Date: Nov. 11, 2010

(54) GENES ASSOCIATED WITH CHEMOTHERAPY RESPONSE AND USES THEREOF

(76) Inventors: Hongyue Dai, Chestnut Hill, MA (US); Chunsheng Zhang, West Roxbury, MA (US); Andrey Loboda, Philadelphia, PA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/307,114

(22) PCT Filed: Jun. 28, 2007
(86) PCT No.: PCT/US07/15025

§ 371 Date: Mar. 15, 2010

Related U.S. Application Data
(60) Provisional application No. 60/818,262, filed on Jun. 30, 2006.

(51) Int. Cl. A61K 49/00; C12Q 1/68; C40B 30/00; A61K 31/713; A61K 31/7105; A61K 38/02; A61K 39/395; A61K 31/513; A61K 31/675; A61K 31/337; A61K 31/7048; A61K 31/282; C12N 5/07; C12N 5/071; C40B 40/08; A61K 31/7088; A61P 35/00

(52) U.S. Cl. 424/9.1; 435/6; 506/7; 514/44 A; 514/44 R; 514/19.3; 424/174.1; 514/274; 514/90; 514/449; 514/27; 514/492; 435/375; 435/366; 506/17

(57) ABSTRACT

The invention provides molecular markers that are associated with responsiveness of a cancer patient to a chemotherapy treatment, and methods and computer systems for determining such responsiveness based on measurements of these molecular markers. The present invention also provides methods and compositions for enhancing the efficacy of chemotherapies in patients by modulating the expression or activity of genes encoding these molecular markers and/or their encoded proteins.

This application claims the benefit under 35 U.S.C. §119(e) of U.S. Provisional Patent Application No. 60/818,262, filed on Jun. 30, 2006, which is incorporated by reference herein in its entirety.

### 1. FIELD OF THE INVENTION

The invention relates to molecular markers that are associated with responses to chemotherapies in a patient, and methods and computer systems for determining such responses based on measurements of these molecular markers. The present invention also relates to methods and compositions for enhancing the efficacy of chemotherapies in patients by modulating the expression or activity of genes encoding these molecular markers and/or their encoded proteins.

### 2. BACKGROUND OF THE INVENTION

Chemotherapy is an important modality in treating many types of cancers. A large and growing variety of potent chemotherapeutic agents targeting cancer cells by various mechanisms have been developed and can be used individually or in combination. With the help of such a growing menu of chemotherapeutic agents, the disease-free survival and overall survival of cancer patients have been significantly improved for many types of cancers. However, not all cancer patients are responsive to all available chemotherapy treatments. Most chemotherapeutic agents cause severe side effects, such as anemia, infections and sepsis (sometimes lethal) due to immune suppression, hemorrhage, and hepatotoxicity. Chemotherapy treatments generally are also physically exhausting for patients, and are often associated with high costs. Therefore, determining whether a cancer patient should receive chemotherapy and choosing the appropriate chemotherapy are often important parts of medical intervention. Traditionally, chemotherapy is prescribed to cancer patients based on their disease prognosis and risk of side effects. For example, in cases of breast cancer, such prognostic and predictive factors as age, tumor size, axillary lymph node status, histological tumor type, pathological grade and hormone receptor status have been used to evaluate whether a patient may benefit from chemotherapy treatments.

In the past several years, gene expression signatures of cancer cells have been found to provide more accurate disease prognosis and/or prediction of chemotherapy responsiveness than traditional clinical factors. Gene markers that are informative for predicting breast cancer outcome have been disclosed (see, e.g., United States Patent Publication 20030224374; United States Patent Publication 20040058340; van't Veer et al., 2001, Nature 415:530; van de Vijver et al., 2002, N. Engl. J. Med. 347:1999). Expression profiles of such gene markers, e.g., a 70-gene set, was found to be capable of predicting the likelihood of the occurrence of metastases within five years of initial diagnosis in breast cancer patients. It was found that a prognosis based on expression profiles of the gene markers outperforms that based on traditional clinical factors. The 70-gene set was also found to be capable of predicting whether a patient should be treated with systemic therapies such as chemotherapy and hormonal therapy (see United States Patent Publication 20040058340).

The 70-gene marker set has also been found to be useful for predicting the responsiveness of a breast cancer patient to chemotherapy in certain patient subgroups (see, e.g., Dai et al., US 2004-0058340, published Mar. 25, 2004). Among patients whose gene expression profile indicates poor prognosis, a patient's responsiveness to chemotherapy depends not only on the patient's ER level, but also on the change of the ER level with age. It discloses that patients who show high ER level at an earlier age (thus a high ER/AGE) show little response to chemotherapy, whereas patients who show high ER level at later age (thus a low ER/AGE) show increased response to chemotherapy.

Pawitan et al. (Pawitan et al., 2005, Breast Cancer Res. 7:R953-964) reported identification of gene expression signatures that are associated with prognosis and response to adjuvant therapies. Gene expression profiles of tumor samples from 159 population-derived breast cancer patients were analyzed using hierarchical clustering, and a set of 64 genes was identified. The 64-gene set was found to be able to distinguish three subclasses of patients: patient who did well with therapy, patients who did well without therapy, and patients who failed to benefit from given therapy.

Wang et al. investigated the gene expression patterns of chemoresistance to thymidylate synthase (TS) inhibitors Raltitrexed (TDX) and 5-fluorouracil (5-FU) in a panel of 5 matched cancer cell lines (Wang et al., 2001, Cancer Res. 61:5505-10). By comparing the expression profiles of resistant cell lines and their respective chemosensitive parent cell lines, Wang et al. have found 28 genes whose expression levels were altered >1.5-fold among resistant cells, with 2 genes (TS and YES1) consistently higher in the panel.

Duan et al. disclosed identification of genes involved in a paclitaxel resistance phenotype (Duan et al., 2005, Cancer Chemotherapy and Pharmacology 55:277-285). Affymetrix HG-U95Av2 microarrays were used to quantify gene expression differences between the resistant and sensitive cell lines. Three paclitaxel-resistant human ovarian and breast cancer cell lines were established from drug-sensitive patental cell lines. Eight genes were identified to be significantly over-expressed in the three drug-resistant cell lines, including multi-drug resistant gene 1 (MDR1), and three genes were identified to be significantly under-expressed in the three drug-resistant cell lines.

Chang et al. disclosed evaluating tumor response to neoadjuvant docetaxel treatment in breast cancer patient based on expression profiles (Chang et al., 2003, Lancet. 362:362-369). Differential patterns of expression of 92 genes were found to correlate with docetaxel response. Among these genes, a higher expression of genes involved in cell cycle, cytoskeleton, adhesion, protein transport, protein modification, transcription, and stress or apoptosis was found to be associated with sensitive tumors, whereas increased expression of some transcriptional and signal transduction genes was found to be associated with resistant tumors. Chang et al. disclosed that the molecular patterns of the residual cancers after three months of docetaxel treatment were found to be strikingly similar, independent of initial sensitivity or resistance (Chang et al., 2005, J Clin Oncol 23:1169-1177). They concluded that this may indicate selection of a residual and resistant subpopulation of cells. The gene expression pattern was populated by genes involved in cell cycle arrest at G2M (e.g., mitotic cyclins and cdc2) and survival pathways involving the mammalian target of rapamycin. The authors state that these genes may be therapeutic targets that could lead to improved treatment.

Luker et al. (Luker et al., 2001, Cancer Res. 61:6540-6547) reported identification of interferon regulatory factor 9 (IRF9) as a positive regulator of resistance to anti-microtubule agents such as paclitaxel in breast cancer cells. Luker et al. showed that several proteins in the type I IFN regulated pathway were over-expressed in paclitaxel-resistant breast tumor cell lines derived from the MCF-7 cell line and in untreated breast tumor samples and uterine tumor samples.

Einav et al. (Einav et al., 2005, Oncogen 24:6367-75) reported an analysis of gene expression data of various cancers, including gene expression data of childhood acute lymphoblastic leukemia (ALL) samples (Yeoh et al., 2002, Cancer Cell 1:133-143), gene expression data of breast cancer samples (van't Veer et al., 2002, Nature 415:530-536), and gene expression data of ovarian cancer samples (Welsh et al., 2001, Proc. Natl. Acad. USA 98:1176-81), among others. They discovered that a group of about 30 correlated genes, containing mainly genes in the interferon response pathway, are over-expressed in certain subclasses of ALL samples, breast cancer samples and ovarian cancer samples.

Spentzos et al. reported a 93-gene signature that can be used to prognose chemotherapy responsiveness in epithelial ovarian cancer patients (Spentzos et al., 2005, J. Clinic. Oncology 23:7911-7918).

WO 2005/100606 disclosed gene sets useful in predicting the response of cancer, e.g. breast cancer patients to chemotherapy. WO 2005/100606 also disclosed a multi-gene RNA analysis based cancer test which can be used for predicting patient response to chemotherapy.

Discussion or citation of a reference herein shall not be construed as an admission that such reference is prior art to the present invention.

### 3. SUMMARY OF THE INVENTION

The invention provides a method for predicting the responsiveness of a mammalian patient having a cancer to a chemotherapy regimen, comprising predicting said patient as (a) responsive to said chemotherapy regimen, if expression and/or activity of one or more gene products in a cell sample taken from said patient is not up-regulated relative to a reference population of individuals of the same species as said patient; or (b) non-responsive to said chemotherapy regimen, if expression and/or activity of said one or more gene products is up-regulated relative to said reference population, wherein said one or more gene products comprise respectively products of one or more different genes selected from the group consisting of genes corresponding to SEQ ID NOsNOs:1-39 or respective functional equivalents thereof. In one embodiment, the method further comprises prior to said step of predicting a step of determining whether expression and/or activity of said one or more gene products is up-regulated as relative to said reference population of individuals.

In some embodiments, said step of determining is carried out by a method comprising determining one or more chemotherapy response scores (CR scores) based on measurements of at least said one or more gene products in said cell sample, wherein said one or more CR scores indicate whether expression and/or activity of said one or more first gene products is up-regulated as compared to individuals in said reference population.

In one embodiment, said step of determining one or more CR scores is carried out by a method comprising determining a CR score that is an average of said measurements of said one or more gene products, wherein said patient is predicted as responsive if said average is less or equal to a predetermined threshold value or as non-responsive if said average is greater than said predetermined threshold value.

In another embodiment, said step of determining one or more CR scores is carried out by a method comprising determining a CR score that is a measurement of a gene product of a gene having the greatest expressive range among said different genes selected from the group consisting of genes having SEQ ID NOsNOs:1-19 or among said different genes selected from the group consisting of genes having SEQ ID NOsNOs:20-39, wherein said patient is predicted as responsive if said measurement is less or equal to a predetermined threshold value or as non-responsive if said measurement is greater than said predetermined threshold value.

In still another embodiment, said step of determining one or more CR scores is carried out by a method comprising (a1) comparing a marker profile comprising said measurements of said one or more gene products with a responsive template and/or a non-responsive template, said responsive template comprising measurements of said one or more gene products representative of measurements of said one or more genes products in a plurality of patients being responsive to said chemotherapy regimen, and said non-responsive template comprising measurements of said one or more gene products representative of measurements of said plurality of genes products in a plurality of patients being non-responsive to said chemotherapy regimen; and (a2) determining a first degree of similarity between said marker profile and said responsive template and/or a second degree of similarity between said marker profile and said non-responsive template, wherein said first and second degrees of similarity are said one or more CR scores, and wherein said patient is (b1) predicted to be responsive if said first degree of similarity is greater than said second degree of similarity or if said first degree of similarity is greater than a predetermined threshold or (b2) predicted to be non-responsive if said first degree of similarity is no greater than said second degree of similarity or if said second degree of similarity is no greater than said predetermined threshold. In one embodiment, each said degree of similarity is represented by a correlation coefficient between said marker profile and said respective template. In one embodiment, the measurement of each gene product in said responsive template is an average of the measurements of said gene product in a plurality of responsive patients, and wherein the measurement of each gene product in said non-responsive template is an average of the measurements of said gene product in a plurality of non-responsive patients.

In another embodiment, said step of determining one or more CR scores is carried out by a method comprising using a chemotherapy response classifier selected from the group consisting of an artificial neural network (ANN) classifier and a support vector machine (SVM) classifier, wherein said chemotherapy response classifier receives an input comprising a marker profile comprising said measurements of said one or more gene products and provides an output comprising said one or more CR scores. In one embodiment, said chemotherapy response classifier is trained with training data from a plurality of training cancer patients, wherein said training data comprise for each patient of said plurality of training cancer patients (i) a training marker profile comprising measurements of said plurality of gene products in a cell sample taken from said training patient; and (ii) data indicating whether said training patient is responsive to said treatment regimen.

In still another embodiment, the method comprises determining one or more CR scores that indicates in which percentile said measurements of said one or more gene products fall in the said reference population, wherein said patient is predicted to be non-responsive if said one or more CR scores indicate that said measurements of said one or more gene products fall in the Y1 percentile in said reference population, wherein Y1 percentile=60 percentile, 70 percentile, 80 percentile, or 90 percentile, or is predicted to be responsive if said one or more CR scores indicate that said measurements of said one or more gene products fall in the Y2 percentile in said reference population, wherein Y2 percentile=10 percentile, 20 percentile, 30 percentile, or 40 percentile.

In one embodiment, said measurements of one or more gene products are measurements of abundance levels of gene transcripts.

In another embodiment, said measurements of one or more gene products are measurements of abundance levels of proteins.

In a specific embodiment, said chemotherapy regimen comprises administration of a chemotherapy drug selected from the group consisting of 5-fluorouracil, CMF combination consisting of cyclophosphamide, methotrexate, and 5-fluorouracil, paclitaxel, etoposide, and carboplatin.

In one embodiment, said one or more gene products are respectively products of the genes selected from the group consisting of genes having SEQ ID NOsNOs:1-39. In another embodiment, said one or more gene products are of at least N or are all of the different genes selected from the group consisting of genes having SEQ ID NOsNOs:1-39, wherein N=2, 3, 4, 5, 10, 15, 20, 25, 30, or 35. In still another embodiment, said one or more gene products are of at least N, or are all of the different genes selected from the group consisting of genes having SEQ ID NOsNOs:1-19, wherein N=2, 3, 4, 5, 10, or 15. In still another embodiment, said one or more gene products are of at least N, or are all of the different genes selected from the group consisting of genes having SEQ ID NOs:20-39, wherein N=2, 3, 4, 5, 10, or 15. In still another embodiment, said one or more gene products comprises gene products of (i) at least N, or are all of the different genes selected from the group consisting of genes having SEQ ID NOs:1-19, wherein N=2, 3, 4, 5, 10, or 15 and (ii) at least M, or are all of the different genes selected from the group consisting of genes having SEQ ID NOs:20-39, wherein M=2, 3, 4, 5, 10, or 15.

In one embodiment, the chemotherapy regimen is an adjuvant chemotherapy regimen, and wherein a prediction of a patient as responsive to said chemotherapy regimen indicates non-occurrence of metastases or survival within a first predetermined period of time after initial diagnosis in said patient treated with said chemotherapy regimen, and wherein a prediction of a patient as non-responsive to said chemotherapy regimen indicates occurrence of metastases or non-survival within a second predetermined period of time in said patient treated with said chemotherapy regimen. In another embodiment, said chemotherapy regimen is a primary chemotherapy regimen, and a prediction of a patient as responsive to said chemotherapy regimen indicates (i) a reduction in tumor size or number of cancer cells and/or (ii) non-occurrence of metastases or survival within a first predetermined period of time after initial diagnosis in said patient treated with said chemotherapy regimen, and wherein a prediction as responsive to said chemotherapy regimen indicates (iii) a lack of reduction in tumor size or number of cancer cells and/or (iv) occurrence of metastases or non-survival within a second predetermined period of time in said patient treated with said chemotherapy regimen. The first period of time and said second periods of time can be the same, e.g., each 3, 5, 7, 10, or 12 years.

In another embodiment, said patient has been determined to have a poor prognosis, wherein a poor prognosis indicates occurrence of metastases or non-survival within a third predetermined period of time (e.g., 3, 5, 7 or 10 years) in said patient untreated with any chemotherapy for said cancer.

In one embodiment, said measurement of each said gene product is a relative level of said gene product in said cell sample versus level of said gene product in a reference sample, represented as a log ratio.

In one embodiment, said reference sample is selected from the group consisting of a sample comprising a pool of cancer cells obtained from a plurality of patients having said cancer, a sample of cells of a non-cancerous cell line of cells of the same type of tissue as said cancer, and a sample of cells of a cell line of said cancer.

In a preferred embodiment, said patient is a human patient.

In one embodiment, said cancer is breast cancer. In another embodiment, said cancer is ovarian cancer.

The invention also provides a method for assigning a treatment regimen for a patient having a cancer, comprising (i) predicting whether said patient is responsive or non-responsive to a chemotherapy regimen using the method described above; and (ii) if said patient is determined to be responsive to said chemotherapy regimen, assigning said patient a treatment regimen that comprises said chemotherapy regimen; or if said patient is determined to be non-responsive to said chemotherapy regimen, assigning said patient (ii1) a treatment regimen that does not comprise said chemotherapy regimen or (ii2) a treatment regiment comprising (A) said chemotherapy regimen and (B) one or more agents that reduce the expression and/or activity level of said one more gene products.

The invention also provides a method for enrolling a plurality of cancer patients for a clinical trial of a chemotherapy regimen, comprising (i) determining whether each patient in said plurality is responsive or non-responsive to said chemotherapy regimen using the method described above; and (ii) assigning each patient who is predicted to be responsive to one patient group and each patient who is predicted to be non-responsive to another patient group, at least one of said patient group being enrolled in said clinical trial.

In a preferred embodiment, the above described methods are computer-implemented methods.

The methods of the invention can further comprise obtaining said measurements of said one or more gene products by a method comprising measuring said plurality of gene products of said cell sample taken from said patient.

In one embodiment, the method further comprises obtaining measurement of abundance level of each said gene transcript by a method comprising contacting a positionally-addressable microarray with nucleic acids from said cell sample or nucleic acids derived therefrom under hybridization conditions, and detecting the amount of hybridization that occurs, said microarray comprising one or more polynucleotide probes complementary to a hybridizable sequence of each said gene transcript or a nucleic acid derived thereof. In one embodiment, measurement of abundance level of each said gene transcript by a method comprising measuring the transcript level of said gene using quantitative reverse transcriptase PCT (qRT-PCR).

The invention also provides a computer system comprising a processor, and a memory coupled to said processor and encoding one or more programs, wherein said one or more programs cause the processor to carry out any one of the method of described above. The invention also provides a computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out any one of the method of described above.

The invention also provides a method for treating a patient having a cancer, comprising administering to said patient (a) one or more agents that is capable of reducing the expression and/or activity of one or more different genes selected from the group consisting of genes having SEQ ID NOs:1-39 or respective functional equivalents thereof and/or their encoded proteins, and (b) a chemotherapy regimen, wherein said patient is predicted to be non-responsive to said chemotherapy regimen as a result of over-expression of said one or more different genes. In one embodiment, said one or more different genes consist of at least N or all of the different genes selected from the group consisting of genes having SEQ ID NOs:1-39, wherein N=2, 3, 4, 5, 10, 15, 20, 25, 30, or 35. In another embodiment, said one or more different genes consist of at least N or all of the different genes selected from the group consisting of genes having SEQ ID NOs:1-19, wherein N=2, 3, 4, 5, 10, or 15. In still another embodiment, said one or more different genes consist of at least N or all of the different genes selected from the group consisting of genes having SEQ ID NOs:20-39, wherein N=2, 3, 4, 5, 10, or 15. In still another embodiment, said one or more different gene are of (i) at least N or all of the different genes selected from the group consisting of genes having SEQ ID NOs:1-19, wherein N=2, 3, 4, 5, 10, or 15; and (ii) at least K or all of the different genes selected from the group consisting of genes having SEQ ID NOs:20-39, wherein K=2, 3, 4, 5, 10, or 15.

In some embodiments, said one or more agents comprise a substance selected from the group consisting of siRNA, antisense nucleic acid, ribozyme, and triple helix forming nucleic acid, each being capable of reducing the expression of one or more of said one or more different genes. In a preferred embodiment, said one or more agents comprise an siRNA targeting said one or more different genes. In one embodiment, said one or more different genes consist of at least L different genes, wherein L=2, 3, 4, 5, 10, or 15.

In some other embodiments, said one or more agents comprise a substance selected from the group consisting of antibody, peptide, and small molecule, each is capable of reducing the activity of one or more of proteins encoded by said one or more different genes.

In some embodiments, the method further comprises determining a transcript level of each of said one or more different genes. In one embodiment, said determining each said transcript level is carried out by a method comprising measuring the transcript level of said gene using one or more polynucleotide probes, each of said one or more polynucleotide probes comprising a nucleotide sequence complementary to a hybridizable sequence in said transcript of said gene or a nucleic acid derived thereof. In one embodiment, said one or more polynucleotide probes are polynucleotide probes on a microarray. In another embodiment, said determining each said transcript level is carried out by a method comprising measuring the transcript level of said gene using quantitative reverse transcriptase PCT (qRT-PCR).

In a specific embodiment, said chemotherapy regimen comprises administering a chemotherapy drug selected from the group consisting of 5-fluorouracil, CMF combination consisting of cyclophosphamide, methotrexate, and 5-fluorouracil, paclitaxel, etoposide, carboplatin.

In the methods of treating a cancer patient, the patient can be a human patient. The cancer can be breast cancer or ovarian.

The invention also provides a method for modulating sensitivity of a cell to a chemotherapeutic drug, comprising contacting said cell with one or more agents, said one or more agents being capable of reducing the expression and/or activity of one or more different genes selected from the group consisting of genes having SEQ ID NOs:1-39 or respective functional equivalents thereof and/or the their encoded proteins. The invention also provides a method for modulating growth of a cell, comprising contacting said cell with (a) one or more agents, said one or more agents being capable of reducing the expression and/or activity of one or more different genes selected from the group consisting of genes having SEQ ID NOs:1-39 or respective functional equivalents thereof and/or the their encoded proteins; and (b) a sufficient amount of a chemotherapeutic drug. The method can be carried out in vivo. The method can also be carried out in vitro.

In one embodiment, said one or more different genes consist of at least N or all of the different genes selected from the group consisting of genes having SEQ ID NOs:1-39, wherein N=2, 3, 4, 5, 10, 15, 20, 25, 30, or 35. In another embodiment, said one or more different genes consist of at least N or all of the different genes selected from the group consisting of genes having SEQ ID NOs:1-19, wherein N=2, 3, 4, 5, 10, or 15. In still another embodiment, said one or more different genes consist of at least N or all of the different genes selected from the group consisting of genes having SEQ ID NOs:20-39, wherein N=2, 3, 4, 5, 10, or 15. In still another embodiment, said one or more different gene are of (i) at least N or all of the different genes selected from the group consisting of genes having SEQ ID NOs:1-19, wherein N=2, 3, 4, 5, 10, or 15; and (ii) at least K or all of the different genes selected from the group consisting of genes having SEQ ID NOs:20-39, wherein K=2, 3, 4, 5, 10, or 15.

In one embodiment, said one or more agents comprise a substance selected from the group consisting of siRNA, antisense nucleic acid, ribozyme, and triple helix forming nucleic acid, each is capable of reducing the expression of one or more of said one or more different genes. In one embodiment, said one or more agents comprise an siRNA targeting said one or more different genes. In one embodiment, said one or more different genes consist of at least L different genes, wherein L=2, 3, 4, 5, 10, or 15.

In another embodiment, said one or more agents comprise a substance selected from the group consisting of antibody, peptide, and small molecule, each is capable of reducing the activity of one or more of proteins encoded by said one or more different genes.

In some embodiments, the method further comprises determining a transcript level of each of said one or more different genes. In one embodiment, said determining each said transcript level is carried out by a method comprising measuring the transcript level of said gene using one or more polynucleotide probes, each of said one or more polynucleotide probes comprising a nucleotide sequence complementary to a hybridizable sequence in said transcript of said gene or a nucleic acid derived thereof. In one embodiment, said one or more polynucleotide probes are polynucleotide probes on a microarray. In another embodiment, said determining each said transcript level is carried out by a method comprising measuring the transcript level of said gene using quantitative reverse transcriptase PCT (qRT-PCR).

In a specific embodiment, said chemotherapy regimen comprises administering a chemotherapy drug selected from the group consisting of 5-fluorouracil, CMF combination consisting of cyclophosphamide, methotrexate, and 5-fluorouracil, paclitaxel, etoposide, carboplatin.

In the methods of for modulating sensitivity of a cell to a chemotherapeutic drug, the cell is a human cell. The cell can be a breast cancer cell or an ovarian cancer cell.

The invention also provides a method of identifying an agent that is capable of modulating sensitivity of a cell to the growth inhibitory effect of a chemotherapeutic drug, said method comprising comparing a first growth inhibitory effect of said chemotherapeutic drug on cells expressing said gene in the presence of a candidate agent with a second growth inhibitory effect of said chemotherapeutic drug on cells expressing said gene in the absence of said agent, wherein said agent is capable of reducing the expression and/or activity of a gene selected from the group consisting of genes having SEQ ID NOs:1-39 or respective functional equivalents thereof and/or its encoded protein, wherein a difference in said first inhibitory effect and said second growth inhibitory effect identifies said agent as capable of modulating sensitivity of said cell to the growth inhibitory effect of said chemotherapeutic drug. In one embodiment, the method further comprises (a) contacting a first cell expressing said gene with said chemotherapeutic drug in the presence of said agent and measuring said first growth inhibitory effect; and (b) contacting a second cell expressing said gene with said chemotherapeutic drug in the absence of said agent and measuring said second growth inhibitory effect. The method can be carried out in vivo, e.g., on human or non-human patients. The method can also be carried out in vitro, e.g., on cells of a cell culture.

In one embodiment, said agent comprises a substance selected from the group consisting of siRNA, antisense nucleic acid, ribozyme, and triple helix forming nucleic acid, each reducing the expression of said genes.

In another embodiment, said one or more agents comprise a substance selected from the group consisting of antibody, peptide, and small molecule, each reducing the activity of one or more of proteins encoded by said one or more different genes in said patient.

In a specific embodiment, said chemotherapy regimen comprises administering a chemotherapy drug selected from the group consisting of 5-fluorouracil, CMF combination consisting of cyclophosphamide, methotrexate, and 5-fluorouracil, paclitaxel, etoposide, carboplatin.

In the methods, said cell can a breast cancer cell or an ovarian cancer cell.

The invention also provides a microarray comprising for each of one or more different genes selected from the group consisting of genes having SEQ ID NOs:1-39 or respective functional equivalents thereof, one or more polynucleotide probes complementary and hybridizable to a sequence in said gene, wherein polynucleotide probes complementary and hybridizable to said genes constitute at least 50%, 60%, 70%, 80% or 90% of the probes on said microarray. In one embodiment, said one or more different genes consist of at least N or all of the different genes selected from the group consisting of genes having SEQ ID NOs:1-39, wherein N=2, 3, 4, 5, 10, 15, 20, 25, 30, or 35. In another embodiment, said one or more different genes consist of at least Nor all of the genes selected from the group consisting of genes having SEQ ID NOs:1-19, wherein N=2, 3, 4, 5, 10, 15. In still another embodiment, said one or more different genes consist of at least Nor all of the different genes selected from the group consisting of genes having SEQ ID NOs:20-39, wherein N=2, 3, 4, 5, 10, or 15. In still another embodiment, said one or more different gene are of (i) at least Nor all of the different genes selected from the group consisting of genes having SEQ ID NOs:1-19, wherein N=2, 3, 4, 5, 10, or 15; and (ii) at least N or all of the different genes selected from the group consisting of genes having SEQ ID NOs:20-39, wherein N=2, 3, 4, 5, 10, or 15.

### 4. BRIEF DESCRIPTION OF THE DRAWINGS

FIG. 1. (a) A network (hub #34) enriched for interferon stimulated genes (ISG). (b) The hub genes are highly co-regulated in breast cancer data where the network is derived from. Each row represents a sample, each column represents one gene. A darker shade, which was magenta in the original depiction of FIG. 1b, represents up-regulation, and a lighter shade, which was cyan in the original depiction of FIG. 1b, represents down regulation.

FIG. 2. The expression level of interferon stimulated genes (ISGs) is related to chemotherapy (CMF) sensitivity in breast cancer patients. (a) Patients with low expression of ISGs showed great chemotherapy sensitivity as indicated by the Kaplan-Meier plot of metastasis-free probability between patients who received the treatment (lighter shade, which was red in the original depiction of FIG. 2) vs. no treatment (darker shade, which was blue in the original depiction of FIG. 2). At 10 years after diagnosis of cancer, the treatment boosted the metastasis-free probability from 60% to -95% (log-rank-test P-value 0.3%). (b) Patients with high expression of ISGs showed no chemo-therapy sensitivity. There was essentially no difference in metastasis-free probability between patients with and without chemotherapy (P=75%).

FIG. 3. Exemplary bar chart of number of genes in each P-value bin for 9 hubs. P-value is based on the correlation coefficient between gene expression level and 5-FU drug resistance category in ovarian ex-vivo experiment. Three hubs (#20, 34 and 88) have a significant fraction of members whose base-line expression level correlated with the drug resistance (with P-value of correlation<5%). Two of the 3 hubs (#34 and 88) belong to an ISG pathway.

FIG. 4. Expression of ISGs and their relation with drug resistance in ex-vivo ovarian samples. Left panel: category of 5-FU drug resistance measured by growth inhibition. EDR stands for extreme drug resistance, LDR stands for low drug resistance. The remaining category stands for intermediate. Heatmap: expression of ISGs from hub 34 and hub 88. Each row represents a sample, each column represents one gene. A darker shade, which was magenta in the original heatmap of FIG. 4, represents up-regulation; and a lighter shade, which was cyan in the original heatmap of FIG. 4, represents down regulation. For LDR samples, ISGs are mostly under-expressed compared to the average, whereas for EDR samples, the ISG levels are relatively higher. Top panel: correlation of expression level to drug resistance.

FIG. 5. Fraction of interferon-stimulated-genes (ISGs) correlated with drug resistance in ex-vivo ovarian cancer samples treated with a panel of anti-cancer drugs. The ISGs are relatively specific in reporting the 5-FU drug sensitivity. Results for the following drugs or drug combinations are shown: Taxol; Taxotere; cisplatin (CPLAT); carboplatin (CARBPLT); cisplatin+gemcitabine (CPG); cyclophosphamide (FOURHC); Doxil (DOXILR); etoposide (ETOP); gemcitabine (GMCB); Topotecan (TOPOR); carboplatin+taxol (CARTXn); cisplatin+cyclosporin A (CPCSAn); cisplatin+verapamil (CPVERn); Doxil (DOXILPCI); doxil+cyclosporin A (DXLCAn); 5-FU (FIVEFUn); hexamethylmelamine (PMMn); taxol+cyclosporin (TAXCAn); TOPOTECAN (TOPOPn).

FIG. 6 illustrates an exemplary embodiment of a computer system for implementing the methods of this invention.

### 5. DETAILED DESCRIPTION OF THE INVENTION

The invention provides molecular markers, i.e., genes, the expression levels of which can be used for evaluating the responsiveness of a cancer patient to chemotherapy. The identities of these markers and the measurements of their respective gene products, e.g., measurements of levels (abundances) of their encoded mRNAs or proteins, can be used to develop a chemotherapy responsiveness classifier that discriminates sensitivity from resistance to one or more chemotherapeutic agents based on measurements of such gene products in a sample from a patient. As used herein, the term “gene product” includes mRNA transcribed from the gene and protein encoded by the gene.

As used herein, chemotherapy in the context of a cancer patient refers to the treatment, preferably systemic, of the cancer patient with one or more anticancer drugs. Depending on the type and stage of the cancer, the chemotherapy can be adjuvant chemotherapy or primary chemotherapy. Adjuvant chemotherapy of a cancer patient refers to chemotherapy of a patient whose primary tumor has been surgically removed and who exhibits no evidence that cancer remains. Primary chemotherapy, also called neoadjuvant chemotherapy or induction chemotherapy, refers to chemotherapy prior to a definitive surgical and/or other local therapeutic (e.g. radiotherapeutic) procedure. Primary chemotherapy can be used either prior to surgery or radiation to reduce the tumor size or as the main treatment, e.g., for treating patients whose cancer is inoperable and/or has become metastatic. Primary chemotherapy is used in treating some patients with certain cancers, such as specific types of lymphomas, some small cell lung cancers, and locally advanced breast cancer. The appropriate dose and/or schedule of chemotherapy treatment of a cancer patient can be determined by a person skilled in the art. In preferred embodiments, the chemotherapy treatment is carried out according to standard medical practice for treating the particular cancer. Chemotherapy treatment of a patient can begin at any time after the initial diagnosis.

A patient is said to be responsive or sensitive to a chemotherapy treatment (“responsive patient” or “responder”) if the chemotherapy treatment confers benefit to the patient, whereas a patient is said to be non-responsive or resistant to a chemotherapy treatment (“non-responsive patient” or “non-responder”) if the chemotherapy treatment fails to confer benefit to the patient. Whether a patient is benefited can be determined clinically by a person skilled in the art. For example, benefits to a cancer patient include but are not limited to one or more of the following: reduction of the size of the tumor and/or quantity of tumor cells in the patient, metastasis-free survival within a predetermined period of time after initial diagnosis, e.g., a period of 1, 2, 3, 4, 5 or 10 years, or overall survival within a predetermined period of time after initial diagnosis, e.g., a period of 1, 2, 3, 4, 5 or 10 years. Thus, in cases of adjuvant chemotherapy, in one embodiment, a patient treated by an adjuvant chemotherapy regimen is said to be responsive if no metastases occurs within a predetermined period of time after initial diagnosis, e.g., a period of 1, 2, 3, 4, 5 or 10 years, whereas the patient is said to be non-responsive if metastases occurs within a predetermined period of time, e.g., a period of 1, 2, 3, 4, 5 or 10 years. In another embodiment, a patient treated by an adjuvant chemotherapy regimen is said to be responsive if the patient survives within a predetermined period of time after initial diagnosis, e.g., a period of 1, 2, 3, 4, 5 or 10 years, whereas the patient is said to be non-responsive if the patient does not survive within a predetermined period of time, e.g., a period of 1, 2, 3, 4, 5 or 10 years. In cases of primary chemotherapy, in one embodiment, a patient treated by a primary chemotherapy regimen is said to be responsive if a reduction in tumor size or number of cancer cells occurs and/or no metastases occurs or the patient survives within a predetermined period of time after initial diagnosis, e.g., a period of 1, 2, 3, 4, 5 or 10 years, whereas the patient is said to be non-responsive if no reduction in tumor size or number of cancer cells occurs and/or metastases occurs or the patient does not survive within a predetermined period of time after initial diagnosis, e.g., a period of 1, 2, 3, 4, 5 or 10 years. For primary chemotherapy, local surgical or radiation treatment of the primary tumor may also be performed after the chemotherapy treatment.

The invention provides a list of genes that discriminates between responsive patients and non-responsive patients (Table 1, infra). This set of genes is called the chemotherapy response genes. Measurements of gene products of one or more of these genes, as well as of their functional equivalents, can be used for predicting whether a patient having a cancer will be responsive or non-responsive to a treatment regimen of one or more chemotherapeutic agents. A functional equivalent with respect to a gene, designated as gene A, refers to a gene that encodes a protein or mRNA that at least partially overlaps in physiological function in the cell to that of the protein or mRNA encoded by gene A. In particular, prediction of chemotherapy responsiveness in a patient can be carried out by a method comprising determining whether expression and/or activity of the gene product of one or more different genes listed in Table 1, or functional equivalents of such genes, in an appropriate cell sample from the patient, e.g., a tumor sample obtained from the patient, is up-regulated, i.e., increased, relative to a reference population of individuals. The reference population can be a plurality of individuals of the same species as the patient. In a preferred embodiment, the patient is a human patient. In another preferred embodiment, the reference population comprises a plurality of patients having the same type of cancer. Preferably, the reference population comprises both responsive patients and non-responsive patients. The reference population can comprise at least 10, 50, 100, 200, or 300 patients. In one embodiment, the expression or activity of a gene product of the patient is determined to be up-regulated if measurement of the expression or activity of the gene product is above a first threshold value. In another embodiment, the expression or activity of a gene product of the patient is determined to be not up-regulated if measurement of the expression or activity of the gene product is not greater than a second threshold value. The first and second threshold value can be the same threshold. In one embodiment, the threshold value is an average value of measurements of the expression or activity of the gene product in the reference population. The first and second threshold value can also be different. In another embodiment, the expression or activity of a gene product of the patient is determined to be up-regulated if the measurement of the expression or activity of the gene product falls in the Y1 percentile in the reference population, i.e., the measurement of the expression or activity of the gene product is greater than Y1% of the individuals in the reference population, where Y1 percentile=60 percentile, 70 percentile, 80 percentile, or 90 percentile. In another embodiment, the expression or activity of a gene product of the patient is determined to be not up-regulated if the measurement of the expression or activity of the gene product falls in the Y2 percentile in the reference population, i.e., the measurement of the expression or activity of the gene product is greater than Y2% of the individuals in the reference population, where Y2 percentile=10 percentile, 20 percentile, 30 percentile, or 40 percentile. In another embodiment, when the one or more genes comprises more than one gene, the above described methods can be adapted by using the sum or average of the measurements of the expression or activity of the gene products.

In some embodiments, a profile of one or more measurements of the expression and/or activity of one or more genes, e.g., at least Nor all, where N=1, 2, 3, 4, 5, 10, 15, 20, 25, 30, or 35; or at least X % of the different genes, where X %=3%, 5%, 10%, 20%, 30%, 40%, 50%, 60%, 70%, 80%, or 90%, in Table 1 is used. Such a profile of measurements is also referred to herein as an “expression profile” or a “marker profile.” In one embodiment, one or more chemotherapy responsiveness scores or indices (“CR scores” or “CR indices”) are determined for a patient based on such an expression profile. The CR scores indicate whether the one or more genes in the marker profile of the patient is increased relative to the reference population. The responsiveness of the patient to the chemotherapy regimen is then determined based on the score or scores.

The invention also provides methods and computer systems for evaluating chemotherapy responsiveness to a chemotherapy regimen in a patient based on a measured marker profile comprising measurements of one or more markers of the present invention, e.g., an expression profile comprising measurements of transcripts of one or more of the genes listed in Table 1, e.g., 1 or at least Nor all different genes, where N=2, 3, 5, 10, 15, 20, 25, 30, or 35, listed in Table 1 or functional equivalents of such genes. The methods and systems of the invention can use a chemotherapy responsiveness classifier for evaluating the responsiveness. The chemotherapy responsiveness classifier can be based on an appropriate pattern recognition method (such as those described in Section 5.2) that receives an input comprising a marker profile and provides an output comprising data, e.g., one or more CR scores, indicating whether the patient is sensitive or resistant to chemotherapy. The chemotherapy response classifier can be constructed with training data from a plurality of cancer patients for whom marker profiles and chemotherapy responsiveness are known. The plurality of patients used for training the chemotherapy response classifier is also referred to herein as the training population. The training data comprise for each patient in the training population (a) a marker profile comprising measurements of gene products of a plurality of genes, respectively, in an appropriate cell sample, e.g., a tumor sample, taken from the patient; and (b) information regarding the patient's responsiveness to chemotherapy (e.g., metastasis free duration under the chemotherapy). Various chemotherapy response classifiers that can be used in conjunction with the present invention are described in Section 5.2., infra. In some embodiments, additional patients having known marker profiles and chemotherapy responsiveness can be used to test the accuracy of the chemotherapy responsiveness classifier obtained using the training population. Such additional patients are also called “the testing population.”

The markers in the marker sets are selected based on their ability to discriminate patients who are responsive to a chemotherapy regimen from patients who are non-responsive to the chemotherapy regimen in a plurality of cancer patients whose chemotherapy responsiveness is known, e.g., the training population. Various methods can be used to evaluate the correlation between marker levels and chemotherapy responsiveness. For example, genes whose expression levels are significantly different across responders and non-responders can be identified using an appropriate method known in the art.

The measurements in the profiles of the gene products that are used can be any suitable measured values representative of the expression levels of the respective genes. The measurement of the expression level of a gene can be direct or indirect, e.g., directly of abundance levels of RNAs or proteins or indirectly, by measuring abundance levels of cDNAs, amplified RNAs or DNAs, proteins, or activity levels of RNAs or proteins, or other molecules (e.g., a metabolite) that are indicative of the foregoing. In one embodiment, the profile comprises measurements of abundances of the transcripts of the marker genes. The measurement of abundance can be a measurement of the absolute abundance of a gene product. The measurement of abundance can also be a value representative of the absolute abundance, e.g., a normalized abundance value (e.g., an abundance normalized against the abundance of a reference gene product) or an averaged abundance value (e.g., average of abundances obtained at different time points or from different tumor cell samples from the patients, or average of abundances obtained using different probes, etc.), or a combination of both. As an example, the measurement of abundance of a gene transcript can be a value obtained using an Affymetrix® GeneChip® to measure hybridization to the transcript.

In another embodiment, the expression profile is a differential expression profile comprising differential measurements of a plurality of transcripts in a sample derived from the patient versus measurements of the plurality of transcripts in a reference sample, e.g., a cell sample of normal cells. Each differential measurement in the profile can be but is not limited to an arithmetic difference, a ratio, or a log(ratio). As an example, the measurement of abundance of a gene transcript can be a value for the transcript obtained using an ink-jet array or a cDNA array in a two-color measurement. In a preferred embodiment, the reference sample comprises target polynucleotide molecules from normal cell samples, e.g., samples of non-cancerous cells. In one embodiment, the non-cancerous cells are from the same kind of biological tissue as the cancerous cells. A biological tissue refers to a collection of interconnected cells that perform a similar function within an organism. In another preferred embodiment, the reference sample comprises target polynucleotide molecules from cell samples from a population of cancer patients.

The invention also provides methods and compositions for enhancing the efficacy of a chemotherapy regimen by modulating the expression and/or activity of one or more of the chemotherapy response genes listed in Table 1 and/or their gene products, and/or by modulating interactions of these genes and/or their gene products with other proteins or molecules, e.g., substrates, in combination of with the chemotherapy regimen. In one embodiment, the expression of one or more of the chemotherapy response genes is reduced to treat a cancer patient in combination with the chemotherapy regimen. Such modulation can be achieved by, e.g., using an siRNA, antisense nucleic acid, ribozyme, and/or triple helix forming nucleic acid that target the chemotherapy response genes. In another embodiment, the activity of one or more chemotherapy response proteins is reduced to enhance the effects of the chemotherapy regimen. Such modulation can be achieved by, e.g., using antibodies, peptide molecules, and/or small molecules that target chemotherapy response proteins. The inventors have discovered that the chemotherapy response genes listed in Table 1 are highly expressed in non-responders as compared to responders, and that reducing the expression levels of these genes enhances the responsiveness of a patient.

The invention also provides methods and compositions for utilizing the chemotherapy response genes, and/or their products for screening for agents that modulate their expression and/or activity and/or modulating their interactions with other proteins or molecules. Agents that modulate expression and/or activity of the chemotherapy response genes can be used in combination with the chemotherapy treatment for treating a non-responsive cancer patient. Such agents include but not limited to siRNA, antisense nucleic acid, ribozyme, triple helix forming nucleic acid, antibody, peptide or polypeptide molecules, and small organic or inorganic molecules.

The present invention also provides methods and compositions for identifying other extra- or intra-cellular molecules, e.g., genes and proteins, which interacts with the chemotherapy response genes, and/or their gene products. The present invention also provides methods and compositions for treating cancer by modulating such extra- or intra-cellular molecules.

A “patient” as used herein is an animal. The patient can be but is not limited to a human, or, in a veterinary context, a non-human animal such as a ruminant, horse, swine, sheep, or a domestic companion animal such as a feline or canine. In a preferred embodiment, the patient is a human patient. Suitable samples that can be used in conjunction with the methods of the present invention include but are not limited to tumor samples, e.g., tumor samples obtained from biopsies. In this application, certain genes (for example, those correspond to SEQ ID NOs 1-39) for human patients are disclosed. A person skilled in the art will be able to determine the corresponding homologs for a non-human animal and use such corresponding homologs to practice the invention in such a non-human animal.

The invention also provides a computer system comprising a processor, and a memory coupled to said processor and encoding one or more programs, wherein said one or more programs cause the processor to carry out a method described herein.

The invention also provides a computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out a method described herein.

### 5.1. Genes Associated with Chemotherapy Response

The invention provides molecular marker sets (of genes) that can be used for evaluating chemotherapy response in a cancer patient. The marker sets comprise one or more markers listed in Table 1. Table 1 lists genes whose gene product can be measured and used to distinguish cancer patients who are sensitive to a chemotherapeutic agent from cancer patients who are resistant to the chemotherapeutic agent. The inventors have discovered that up-regulation of the expression and/or activity of one or more of these genes correlates with resistance to chemotherapy. The genes listed in Table 1 include genes clustered into two different clusters. Genes corresponding to SEQ ID NOs:1-19 belong to one cluster, and genes corresponding to SEQ ID NOs:20-39 belong to another cluster. The genes listed in Table 1 are called the chemotherapy response genes (“CR genes”). The genes listed in Table 1 are particularly useful for evaluating responsiveness of breast cancer or ovarian cancer patients to respective standard chemotherapy regimen, e.g., the CMF combination (consisting of cyclophosphamide, methotrexate, and 5-fluorouracil). For those genes listed in Table 1 that have a GenBank® accession number, the GenBank® accession number is listed. For those genes in Table 1 that do not have a GenBank® Accession No, the Contig ID numbers of the transcript sequences in the Phil Green assembly (Nat Genet 2000 June; 25(2):232-4) is listed. Phil Green's group at the University of Washington assembled ESTs from the Washington University-Merck Human EST Project and CGAP archives. Analysis of expressed sequence tags indicates 35,000 human genes (Nat Genet 2000 June; 25(2):232-4). This assembly, dated Mar. 17, 2000, resulted in 62,064 contigs representing 795,000 ESTs (see web address: www.phrap.org/est_assembly/human/gene_number_methods.html; and wwvv.phrap.org/est_assembly/human/gene_number_methods.html). These contigs have the word “contig” included in their identifiers.

TABLE 1
chemotherapy response genes

| Transcript ID | Gene Symbol | Gene Name | SEQ ID No |
|---|---|---|---|
| NM_002346 | LY6E | lymphocyte antigen 6 complex, locus E | 1 |
| NM_003113 | SP100 | nuclear antigen Sp100 | 2 |
| Contig43645_RC | LOC129607 | hypothetical protein LOC129607 | 3 |
| NM_002462 | MX1 | myxovirus (influenza virus) resistance | 4 |
| | | 1, interferon-inducible protein p78 | |
| | | (mouse) | |
| NM_002759 | EIF2AK2 | eukaryotic translation initiation factor | 5 |
| | | 2-alpha kinase 2 | |
| NM_004223 | UBE2L6 | ubiquitin-conjugating enzyme E2L 6 | 6 |
| NM_004335 | BST2 | bone marrow stromal cell antigen 2 | 7 |
| NM_005101 | G1P2 | interferon, alpha-inducible protein | 8 |
| | | (clone IFI-15K) | |
| NM_004585 | RARRES3 | retinoic acid receptor responder | 9 |
| | | (tazarotene induced) 3 | |
| Contig25595_RC | KIAA1618 | KIAA1618 | 10 |
| NM_005567 | LGALS3BP | lectin, galactoside-binding, soluble, 3 | 11 |
| | | binding protein | |
| NM_007267 | EVER1 | epidermodysplasia verruciformis 1 | 12 |
| AB037825 | KIAA1404 | KIAA1404 protein | 13 |
| NM_017414 | USP18 | ubiquitin specific protease 18 | 14 |
| M30818 | MX2 | myxovirus (influenza virus) resistance | 15 |
| | | 2 (mouse) | |
| NM_016817 | OAS2 | 2′-5′-oligoadenylate synthetase 2, | 16 |
| | | 69/71 kDa | |
| NM_000308 | PPGB | protective protein for beta- | 17 |
| | | galactosidase (galactosialidosis) | |
| NM_002038 | G1P3 | interferon, alpha-inducible protein | 18 |
| | | (clone IFI-6-16) | |
| AB006746 | PLSCR1 | phospholipid scramblase 1 | 19 |
| AB025254 | TDRD7 | tudor domain containing 7 | 20 |
| Contig1063_RC | | | 21 |
| U72882 | IFI35 | interferon-induced protein 35 | 22 |
| NM_004509 | SP110 | SP110 nuclear body protein | 23 |
| AF026941 | RSAD2 | radical S-adenosyl methionine domain | 24 |
| | | containing 2 | |
| NM_005532 | IFI27 | interferon, alpha-inducible protein 27 | 25 |
| NM_014314 | DDX58 | DEAD (Asp-Glu-Ala-Asp) box | 26 |
| | | polypeptide 58 | |
| NM_006417 | IFI44 | interferon-induced protein 44 | 27 |
| AL137255 | ZC3HDC1 | zinc finger CCCH-type domain | 28 |
| | | containing 1 | |
| NM_006820 | IFI44L | interferon-induced protein 44-like | 29 |
| Contig51660_RC | IFRG28 | 28 kD interferon responsive protein | 30 |
| Contig63102_RC | LGP2 | likely ortholog of mouse D11lgp2 | 31 |
| NM_017523 | BIRC4BP | XIAP associated factor-1 | 32 |
| NM_016816 | OAS1 | 2′,5′-oligoadenylate synthetase 1, | 33 |
| | | 40/46 kDa | |
| NM_017631 | FLJ20035 | hypothetical protein FLJ20035 | 34 |
| Contig47563_RC | FLJ31033 | hypothetical protein FLJ31033 | 35 |
| Contig41538_RC | IFIT3 | interferon-induced protein with | 36 |
| | | tetratricopeptide repeats 3 | |
| NM_017912 | HERC6 | hect domain and RLD 6 | 37 |
| NM_001548 | IFIT1 | interferon-induced protein with | 38 |
| | | tetratricopeptide repeats 1 | |
| NM_001549 | IFIT3 | interferon-induced protein with | 39 |
| | | tetratricopeptide repeats 3 | |

Genes that are not listed in Table 1 but which are functional equivalents of any gene listed in Table 1 can also be used with or in place of the gene listed in the table. A functional equivalent of a gene A refers to a gene that encodes a protein or mRNA that at least partially overlaps in physiological function in the cell to that of the protein or mRNA of gene A.

In various specific embodiments, different numbers and subcombinations of the genes listed in Table 1 are selected as the marker set, whose profile is used in the methods of the invention, as described in Section 5.2., infra. In one embodiment, at least N different genes listed in Table 1 are used, where N=1, 2, 3, 4, 5, 10, 15, 20, 25, 30, or 35. In another embodiment, at least Nor all of the different genes selected from the group consisting of genes having SEQ ID NOs:1-19 are used, where N=1, 2, 3; 4, 5, 10, or 15. In still another embodiment, at least Nor all of the different genes selected from the group consisting of genes having SEQ ID NOs:20-39 are used, where N=1, 2, 3, 4, 5, 10, or 15. In still another embodiment, at least N or all of the different genes selected from the group consisting of genes having SEQ ID NOs:1-19, where N=1, 2, 3, 4, 5, 10, or 15, and at least Mor all of the different genes selected from the group consisting of genes having SEQ ID NOs:20-39, where M=1, 2, 3, 4, 5, 10, or 15, are used. In still another embodiment, one or more of the interferon stimulated genes (ISGs) listed in Table 1 are used. In one embodiment, at least N or all different ISGs listed in Table 1 are used, where N=1, 2, 3, 4, 5, 10.

The invention also provides methods for identifying a set of genes that can be used for evaluating chemotherapy responsiveness in cancer patients. The methods make use of measured expression profiles of a plurality of genes (e.g., measurements of abundance levels of the corresponding gene products) in suitable tumor samples, e.g., tumor cell line or tumor samples from a plurality of patients whose responsiveness to chemotherapy is known. Chemotherapy response markers can be obtained by identifying genes whose expression levels are correlated with responsiveness to chemotherapy. In preferred embodiments, sets of genes co-varying among a population of cancer patients are evaluated to identify those sets whose expression levels correlate with chemotherapy responsiveness in the patients. In other preferred embodiments, sets of genes co-varying among cells of a tumor cell line are evaluated to identify those sets whose expression levels correlate with responsiveness of the tumor cells to chemotherapy treatment.

In one embodiment, co-varying gene sets (also identified as gene networks or hubs in this application) are determined from expression profiles of a plurality of genes (e.g., measurements of abundance levels of the corresponding gene products) in tumor samples from a plurality of patients whose responsiveness to a chemotherapy regimen is known. The plurality of patients comprises both responsive patients and non-responsive patients. Each co-varying gene set is evaluated to determine its association with responsiveness to the chemotherapy. In one embodiment, the plurality of patients is divided into two populations according to the expression level of one or more genes in the co-varying gene set. Patients having high expression level of the one or more genes are assigned to one population (the “high expression population”), and patients having low expression level of the one or more genes are assigned to the other population (the “low expression population”). In one embodiment, the average expression level of all genes in the set is used such that patients having the average expression level above a predetermined threshold level are assigned to the high expression population, and patients having the average expression level below or equal to the predetermined threshold level are assigned to the low expression population. The predetermined threshold level can be a level that best separates the patients according to treatment effect. The effect of the chemotherapy treatment is examined for each patient population. In one embodiment, the metastasis rate is examined to determine whether it is affected by the chemotherapy treatment. In one embodiment, a log-rank-test is performed on one or more suitable clinical parameters that indicate responsiveness or non-responsiveness, e.g., the metastasis free probability as a function of time for patients, with treatment vs. no treatment. The co-varying set is identified as a chemotherapy responsive set if the set has a log-rank-test p-value below a predetermined threshold value in one patient population but not in another patient population, where the populations were stratified based on the level, e.g., average expression level or a representative level, of co-varying genes.

In another preferred embodiment, cell samples of a cancer cell line or from tumor cells grown ex-vivo are used to identify the markers. A plurality of cell samples treated with different doses of a chemotherapeutic agent can be used. The growth inhibitory effect of each drug on the tumor cell is measured. Samples can be categorized into 3 classes for each drug: EDR (extreme drug resistance), MDR (moderate drug resistance), and LDR (low drug resistance). Pairwise correlation coeffients of different genes are calculated. Genes having magnitudes of correlation coefficients above a selected threshold value, e.g., 0.5, are grouped in a co-varying set. Genesets that exhibit significant difference in expression levels in the EDR samples and LDR samples are identified as genesets that can be used to evaluate chemotherapy responsiveness in patients. In one embodiment, genesets containing genes whose expression levels in EDR samples are higher, e.g., at least 1.5 fold higher, than those in LDR samples are identified as genesets that can be used to evaluate chemotherapy responsiveness in patients. In another embodiment, genesets containing genes whose expression levels correlate with low drug resistance, e.g., having a correlation above 0.3, 0.4, or 0.5, are identified as genesets that can be used to evaluate chemotherapy responsiveness in patients.

Methods for grouping genes into co-varying sets are known in the art. See, e.g., U.S. Pat. No. 6,203,987 and U.S. Pat. No. 6,801,859, both of which are incorporated herein by reference in their entireties. The co-varying sets of the present invention can be identified by means of a clustering algorithm (i.e., by means of “clustering analysis”).

The clustering methods and algorithms that can be employed in the present invention include both “hierarchical” or “fixed-number-of groups” algorithms (see, e.g., S-Plus Guide to Statistical and Mathematical Analysis v.3.3, 1995, MathSoft, Inc.: StatSci. Division, Seattle, Wash.). Such algorithms are well known in the art (see, e.g., Fukunaga, 1990, Statistical Pattern Recognition, 2nd Ed., San Diego: Academic Press; Everitt, 1974, Cluster Analysis, London: Heinemann Educ. Books; Hartigan, 1975, Clustering Algorithms, New York: Wiley; Sneath and Sokal, 1973, Numerical Taxonomy, Freeman; Anderberg, 1973, Cluster Analysis for Applications, New York: Academic Press), and include, e.g., hierarchical agglomerative clustering algorithms, the “k-means” algorithm of Hartigan, and model-based clustering algorithms such as mclust by MathSoft, Inc. Preferably, hierarchical clustering methods and/or algorithms are employed in the methods of this invention. In one embodiment, the clustering analysis of the present invention is done using the hclust routine or algorithm (see, e.g., ‘hclust’ routine from the software package S-Plus, MathSoft, Inc., Cambridge, Mass.).

The clustering algorithms used in the present invention operate on a table of data containing gene expression measurements. Specifically, the data table analyzed by the clustering methods comprises an m×k array or matrix wherein m is the total number of conditions or perturbations, i.e., total number of different siRNAs, and k is the number of cellular constituents, e.g., transcripts of genes, measured and/or analyzed.

The clustering algorithms analyze such arrays or matrices to determine dissimilarities between cellular constituents. Mathematically, dissimilarities between cellular constituents i and j are expressed as “distances” I_{i,j}. For example, in one embodiment, the Euclidian distance is determined according to the formula

$${I}_{i , j} = {( {\sum }_{n} {\vert {v}_{i}^{( n )} - {v}_{j}^{( n )} \vert}^{2} )}^{1 / 2} \qquad ( 4 )$$

where vᵢ⁽ⁿ⁾ and vⱼ⁽ⁿ⁾ are the response of cellular constituents i and j respectively to the perturbation n. In other embodiments, the Euclidian distance in Equation 4 above is squared to place progressively greater weight on cellular constituents that are further apart. In alternative embodiments, the distance measure l_{i,j}is the Manhattan distance provide by

$${I}_{i , j} = {\sum }_{n} \vert {v}_{i}^{( n )} - {v}_{j}^{( n )} \vert \qquad ( 5 )$$

In another embodiment, the distance is defined as I_{i,j}=1−r_{, ij}where r_{i,j} is the “correlation coefficient” or normalized “dot product” between the response vectors vᵢ and vⱼ. For example, r_{i,j} is defined by

$${r}_{i , j} = \frac{{v}_{i} \cdot {v}_{j}}{\vert {v}_{i} \vert \vert {v}_{j} \vert} \qquad ( 6 )$$

wherein the dot product vᵢ·vⱼ is defined by

$${v}_{i} \cdot {v}_{j} = {\sum }_{n} {v}_{i}^{( n )} \cdot {v}_{j}^{n} \\ and \vert {v}_{i} \vert = {( {v}_{i} \cdot {v}_{i} )}^{1 / 2} ; and \vert {v}_{j} \vert = {( {v}_{j} \cdot {v}_{j} )}^{1 / 2} \qquad ( 7 )$$

In still other embodiments, the distance measure may be the Chebychev distance, the power distance, and percent disagreement, all of which are well known in the art. In another embodiment, the distance measure is I_{i,j}=1−r_{i,j} with the correlation coefficient which comprises a weighted dot product of the response vector vᵢ and vⱼ. Specifically, in this embodiment, rᵢⱼ is preferably defined by the equation

$${r}_{i , j} = \frac{{\sum }_{n} \frac{{v}_{i}^{( n )} \cdot {v}_{j}^{( n )}}{{σ}_{i}^{( n )} \cdot {σ}_{j}^{( n )}}}{{[ {\sum }_{n} {( \frac{{v}_{i}^{( n )}}{{σ}_{i}^{( n )}} )}^{2} \cdot {\sum }_{n} {( \frac{{v}_{j}^{( n )}}{{σ}_{j}^{( n )}} )}^{2} ]}^{1 / 2}} \qquad ( 8 )$$

where Φᵢ⁽ⁿ⁾ and Φⱼ⁽ⁿ⁾ are the standard errors associated with the measurement of the i'th and j'th cellular constituents, respectively, in experiment n.

The correlation coefficients of Equations 6 and 8 are bounded between values of +1, which indicates that the two response vectors are perfectly correlated and essentially identical, and −1, which indicates that the two response vectors are “anti-correlated” or “anti-sense” (i.e., are opposites). These correlation coefficients are particularly preferable in embodiments of the invention where cellular constituent sets or clusters are sought of constituents which have responses of the same sign.

In other embodiments, it is preferable to identify cellular constituent sets or clusters which are co-regulated or involved in the same biological responses or pathways, but which comprise similar and anti-correlated responses. In such embodiments, it is preferable to use the absolute value of Equation 6 or 8, i.e., |r_{i,j}|, as the correlation coefficient.

In still other embodiments, the relationships between co-regulated and/or co-varying cellular constituents may be even more complex, such as in instances wherein multiple biological pathways (e.g., signaling pathways) converge on the same cellular constituent to produce different outcomes. In such embodiments, it is preferable to use a correlation coefficient rᵢⱼ=rᵢⱼ^{(change)} which is capable of identifying co-varying and/or co-regulated cellular constituents irrespective of the sign. The correlation coefficient specified by Equation 9 below is particularly useful in such embodiments.

$${r}_{i , j}^{change} = \frac{{\sum }_{n} \vert \frac{{v}_{i}^{( n )}}{{σ}_{i}^{( n )}} \vert \vert \frac{{v}_{j}^{( n )}}{{σ}_{j}^{( n )}} \vert}{{[ {\sum }_{n} {( \frac{{v}_{i}^{( n )}}{{σ}_{i}^{( n )}} )}^{2} \cdot {\sum }_{n} {( \frac{{v}_{j}^{( n )}}{{σ}_{j}^{( n )}} )}^{2} ]}^{1 / 2}} \qquad ( 9 )$$

Generally, the clustering algorithms used in the methods of the invention also use one or more linkage rules to group cellular constituents into one or more sets or “clusters.” For example, single linkage or the nearest neighbor method determines the distance between the two closest objects (i.e., between the two closest cellular constituents) in a data table. By contrast, complete linkage methods determine the greatest distance between any two objects (i.e., cellular constituents) in different clusters or sets. Alternatively, the unweighted pair-group average evaluates the “distance” between two clusters or sets by determining the average distance between all pairs of objects (i.e., cellular constituents) in the two clusters. Alternatively, the weighted pair-group average evaluates the distance between two clusters or sets by determining the weighted average distance between all pairs of objects in the two clusters, wherein the weighing factor is proportional to the size of the respective clusters. Other linkage rules, such as the unweighted and weighted pair-group centroid and Ward's method, are also useful for certain embodiments of the present invention (see, e.g., Ward, 1963, J. Am. Stat. Assn 58:236; Hartigan, 1975, Clustering Algorithms, New York: Wiley).

Once a clustering algorithm has grouped the cellular constituents from the data table into sets or cluster, e.g., by application of linkage rules such as those described supra, a clustering “tree” may be generated to illustrate the clusters of cellular constituents so determined.

In a preferred embodiment, tumor samples from a population of M cancer patients are used to identify the markers. Preferably, M is at least 100, 200, or 300. Expression profile of each tumor sample is obtained. Preferably, the population contains both responsive and non-responsive patients. In another preferred embodiment, cell samples of a cancer cell line are used to identify the markers. A plurality of cell samples treated with different doses of a chemotherapeutic agent can be used. The growth inhibitory effect of each drug on the tumor cell is measured. Samples can be categorized into 3 classes for each drug: EDR (extreme drug resistance), MDR (moderate drug resistance), and LDR (low drug resistance). Pairwise correlation coefficients of different genes are calculated. Genes having magnitudes of correlation coefficients above a selected threshold value, e.g., 0.5, are grouped in a co-varying set.

In a specific embodiment, tumor samples from a population of K cancer patients are used, among which N patients received chemotherapy. Microarrays are used for expression profiling. Pairwise correlation coefficients of different genes in the expression profiles are calculated. Genes having magnitudes of correlation coefficients above a predetermined threshold level, e.g., 0.5, are grouped in a co-varying set. A total of S co-varying sets (or hubs) are obtained. The hub expression level of each hub in each cancer sample is then obtained by averaging over genes in the hub. The population of K cancer patients is divided into two subpopulations according to the hub expression level. A threshold that best separate the patients according to treatment effect is found. For example, the threshold can be 20 percentile, 30 percentile 50 percentile, or 80 percentile, which best separates the patients according to treatment effect. Within each subpopulation, the treatment effect is examined by determining whether the metastasis or survival rate is affected by the chemotherapy. In one embodiment, a log-rank-test is performed on the metastasis free probability or probability of survival as a function of time for patients with treatment vs. no treatment. When this search is performed over all K samples, one or more hubs with log-rank-test p-value<0.01 are identified. Among the identified hubs, one or more hubs can be selected.

The selected hubs can also be examined in ex-vivo cancer data sets. Cancer cell line samples are plated ex-vivo and treated by a panel of anticancer drugs. The tumor cell growth inhibition for each drug treatment is measured and samples are categorized into 3 classes for each drug: EDR (extreme drug resistance), MDR (moderate drug resistance), and LDR (low drug resistance). The cancer cell line samples pre-dose of drugs are profiled against the pool of all samples. The expression levels of hub genes are tested by their correlation to the drug resistance categories. The hubs that exhibit significant fraction of members correlated (p-value of correlation<5%) to the growth inhibition by each drug are identified.

The specificity of identified hubs for reporting on the responsiveness to a drug can also be checked. In one embodiment, the correlation between expression level and drug resistance for all tested drugs is calculated. The number (or percentage) of genes in a hub correlated with resistance to a drug can be used as a measure of the specificity of the hub for the drug. In preferred embodiments, a hub for which such a number or percentage for a drug is above a predetermined threshold, e.g., 0.3, 0.4 or 0.5, are identified as specific for the drug.

### 5.2. Methods of Evaluating Responsiveness to Chemotherapy

The invention provides methods for determining the responsiveness of a cancer patient to a chemotherapy regimen using a measured marker profile comprising measurements of one or more of the gene products of genes, e.g., the sets of genes described in Section 5.1., supra. In particular, prediction of chemotherapy responsiveness in a patient can be carried out by a method comprising determining whether expression and/or activity of the gene product of one or more different genes listed in Table 1, or functional equivalents of such genes, in an appropriate cell sample from the patient, e.g., a tumor sample obtained from the patient is up-regulated, i.e., increased, relative to a reference population of individuals, e.g., a plurality of patients having the same type of cancer.

In one embodiment, one or more CR scores or indices are determined for a patient based on the expression levels of one or more of such markers. The CR scores indicate whether the one or more genes in the marker profile of the patient is increased relative to the reference population. The responsiveness of the patient to the chemotherapy, e.g., nonoccurrence of metastases or survival within a predetermined period of time when undergoing a chemotherapy, is then determined based on the score or scores.

In preferred embodiments, the methods of the invention use a chemotherapy response classifier, also called a classifier, for predicting chemotherapy responsiveness to in a patient. The chemotherapy response classifier can be based on an appropriate pattern recognition method that receives an input comprising a marker profile and provides an output comprising data indicating which phase the patient belongs. The chemotherapy response classifier can be trained with training data from a training population of cancer patients. Typically, the training data comprise for each of the cancer patients in the training population a training marker profile comprising measurements of respective gene products of a plurality of genes in a suitable sample taken from the patient and chemotherapy responsiveness information. In a preferred embodiment, the training population comprises both responsive and non-responsive patients.

In preferred embodiments, the chemotherapy response classifier can be based on a classification (pattern recognition) method described below, e.g., profile similarity (Section 5.2.1.1., infra); artificial neural network (Section 5.2.1.2., infra); support vector machine (SVM, Section 5.2.1.3., infra); logic regression (Section 5.2.1.4., infra), linear or quadratic discriminant analysis (Section 5.2.1.5., infra), decision trees (Section 5.2.1.6., infra), clustering (Section 5.2.1.7., infra), principal component analysis (Section 5.2.1.8., infra), nearest neighbor classifier analysis (Section 5.2.1.9., infra). Such chemotherapy response classifiers can be trained with the training population using methods described in the relevant sections, infra.

Various known statistical pattern recognition methods can be used in conjunction with the present invention. A chemotherapy response classifier based on any of such methods can be constructed using the marker profiles and responsiveness data of training patients. Such a chemotherapy response classifier can then be used to evaluate the responsiveness of a cancer patient based on the patient's marker profile. The methods can also be used to identify markers that discriminate between responders and non-responders using such markers. In a preferred embodiment, the methods are used to predict responsiveness of a breast cancer or ovarian cancer patient to a chemotherapy regimen selected from the following: CMF combination (combination of cyclophosphamide, methotrexate, and 5-fluorouracil), 5-FU, paclitaxel (Taxol), etoposide, and carboplatin.

### 5.2.1. Profile Matching

The responsiveness of a cancer patient to a chemotherapy regimen can be evaluated by comparing a marker profile obtained in a suitable sample from the patient with a marker profile that is representative of marker profiles in responsive patients and/or a marker profile that is representative of marker profiles in non-responsive patients. As used herein, a marker profile is said to be representative of marker profiles in a given patient population if the marker profile contains the level of expression and/or activity of one or more genes or gene products that is characteristic of the patients in the population. In preferred embodiments, the marker profile is an average of marker profiles of a plurality of patients in the given patient population. Such a marker profile is also termed a “template profile” or a “template.” A marker profile that is representative of marker profiles in responsive patients is also called a “responsive template”, and a marker profile that is representative of marker profiles in non-responsive patients is also called a “non-responsive template.” The degree of similarity to such a template profile provides an evaluation of the patient's responsiveness to chemotherapy. If the degree of similarity of the patient marker profile and a template profile is above a predetermined threshold, the marker profile of the patient is classified as a marker profile of the class of patients represented by the template, and the patient is predicted to belong to the class of patients.

In one embodiment, the similarity is represented by a correlation coefficient between the patient's profile and the template. In one embodiment, a correlation coefficient above a correlation threshold indicates a high similarity, whereas a correlation coefficient below the threshold indicates a low similarity. Thus, the correlation coefficient can be used as a CR score.

In a specific embodiment, Pᵢ measures the similarity between the patient's profile \vec{y} and a template profile, e.g., the responsive template profile \vec{z}_{R} or the non-responsive template profile \vec{z}_{NR}. Such a coefficient, Pᵢ, can be calculated using the following equation:

Pᵢ=(\vec{z}ᵢ·\vec{y})/(∥\vec{z}ᵢ∥·∥\vec{y}∥)

where i designates the ith template. For example, i is R for the responsive template. Thus, in one embodiment, \vec{y} is classified as a responsive profile, and thus the patient is classified as a responsive patient, if P_{R} is greater than a selected correlation threshold. In another embodiment, \vec{y} is classified as a non-responsive profile, and thus the patient is classified as a non-responsive patient, if P_{NR} is greater than a selected correlation threshold. In preferred embodiments, the correlation threshold is set as 0.3, 0.4, 0.5 or 0.6. In another embodiment, \vec{y} is classified as a responsive profile if P_{R} is greater than P_{NR}, whereas \vec{y} is classified as a non-responsive profile if P_{R} is less than P_{NR}.

In another embodiment, the correlation coefficient is a weighted dot product of the patient's profile \vec{y} and a template profile, in which measurements of each different marker is assigned a weight.

In another embodiment, similarity between a patient's profile and a template is represented by a distance between the patient's profile and the template. In one embodiment, a distance below a given value indicates high similarity, whereas a distance equal to or greater than the given value indicates low similarity.

In one embodiment, the Euclidian distance according to the formula

Dᵢ=∥\vec{y}−\vec{z}ᵢ∥

is used, where Dᵢ measures the distance between the patient's profile \vec{y} and a template profile. In other embodiments, the Euclidian distance is squared to place progressively greater weight on cellular constituents that are further apart. In alternative embodiments, the distance measure Dᵢ is the Manhattan distance provide by

$${D}_{i} = {\sum }_{n} \vert y ( n ) - {z}_{i} ( n ) \vert$$

where y(n) and zᵢ(n) are respectively measurements of the nth marker gene product in the patient's profile \vec{y} and a template profile.

In another embodiment, the distance is defined as Dᵢ=1−Pᵢ, where Pᵢ is the correlation coefficient or normalized dot product as described above.

In still other embodiments, the distance measure may be the Chebychev distance, the power distance, and percent disagreement, all of which are well known in the art.

In one embodiment, the average expression level of the genes in a marker set, e.g., the marker set containing genes having SEQ ID NOs:1-39, or the marker set containing genes having SEQ ID NOs:1-19 or the marker set containing genes having SEQ ID NOs:20-39, is used as the CR score. If the value of the average in a patient sample is above a predetermined threshold value, the patient is classified as a non-responsive patient to chemotherapy treatment using 5-FU, the CMF combination, Paclitaxel, etoposide, or carboplatin, whereas if the value of the average in a patient sample is not greater than the predetermined threshold value, the patient is classified as a responsive patient to such chemotherapy treatment. In another embodiment, the set value of a marker set (see, e.g., U.S. Pat. No. 6, 203,987), e.g., the marker set containing genes having SEQ ID NOs:1-19 or the marker set containing genes having SEQ ID NOs:20-39, is used as the CR score. If the set value in a patient sample is above a predetermined threshold value, the patient is classified as a non-responsive patient to chemotherapy treatment using 5-FU, the CMF combination, Paclitaxel, etoposide, or carboplatin, whereas if the set value in a patient sample is not greater than the predetermined threshold value, the patient is classified as a responsive patient to such chemotherapy treatment. In still another embodiment, the expression level of the gene having the greatest expressive value in a marker set (see, e.g., WO99/58720), e.g., the marker set containing genes having SEQ ID NOs:1-19 or the marker set containing genes having SEQ ID NOs:20-39, is used as the CR score. If the expression level of such gene or genes in a patient sample is above a predetermined threshold value, the patient is classified as a non-responsive patient to chemotherapy treatment using 5-FU, the CMF combination, Paclitaxel, etoposide, or carboplatin, whereas if the expression level of such a gene in a patient sample is not greater than the predetermined threshold value, the patient is classified as a responsive patient to such chemotherapy treatment. In still another embodiment, the average expression level of a subset of genes in a marker set, e.g., at least N or all markers in the marker set containing genes having SEQ ID NOs:1-39, where N=5,10, 20, 30, or at least Mor all markers in the marker set containing genes having SEQ ID NOs:1-19 or in the marker set containing genes having SEQ ID NOs:20-39, where M=5, 10, 15, is used as the CR score. If the average in a patient sample is above a predetermined threshold value, the patient is classified as a non-responsive patient to chemotherapy treatment using 5-FU, the CMF combination, Paclitaxel, etoposide, or carboplatin, whereas if the value of the average in a patient sample is not greater than the predetermined threshold value, the patient is classified as a responsive patient to such chemotherapy treatment. In preferred embodiments, the predetermined threshold value for any one of the above embodiments is an average of the respective measurements in a plurality of training patients. Preferably, the plurality of training patients comprises both responders and non-responders. Thus, the predetermined threshold can be the value of the relevant measurement in the general patient population.

### 5.2.2. Artificial Neural Network

In some embodiments, a neural network is used to classify a patient marker profile. The neural network takes the patient marker profile as an input and generates an output comprising data indicating whether the patient is predicted to be a responsive or a non-responsive patient, e.g., a CR score. A neural network can be constructed for a selected set of molecular markers of the invention. A neural network is a two-stage regression or classification model. A neural network has a layered structure that includes a layer of input units (and the bias) connected by a layer of weights to a layer of output units. For regression, the layer of output units typically includes just one output unit. However, neural networks can handle multiple quantitative responses in a seamless fashion.

In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley & Sons, Inc., New York; and Hastie et al., 2001, The Elements of Statistical Learning, Springer-Verlag, New York.

The basic approach to the use of neural networks is to start with an untrained network, present a training pattern, e.g., marker profiles from training patients, to the input layer, and to pass signals through the net and determine the output, e.g., one or more CR scores indicating chemotherapy responsiveness in the training patients, at the output layer. These outputs are then compared to the target values; any difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, New York.

Three commonly used training protocols are stochastic, batch, and on-line. In stochastic training, patterns are chosen randomly from the training set and the network weights are updated for each pattern presentation. Multilayer nonlinear networks trained by gradient descent methods such as stochastic back-propagation perform a maximum-likelihood estimation of the weight values in the model defined by the network topology. In batch training, all patterns are presented to the network before learning takes place. Typically, in batch training, several passes are made through the training data. In online training, each pattern is presented once and only once to the net.

In some embodiments, consideration is given to starting values for weights. If the weights are near zero, then the operative part of the sigmoid commonly used in the hidden layer of a neural network (see, e.g., Hastie et al., 2001, The Elements of Statistical Learning, Springer-Verlag, New York) is roughly linear, and hence the neural network collapses into an approximately linear model. In some embodiments, starting values for weights are chosen to be random values near zero. Hence the model starts out nearly linear, and becomes nonlinear as the weights increase. Individual units localize to directions and introduce nonlinearities where needed. Use of exact zero weights leads to zero derivatives and perfect symmetry, and the algorithm never moves. Alternatively, starting with large weights often leads to poor solutions.

Since the scaling of inputs determines the effective scaling of weights in the bottom layer, it can have a large effect on the quality of the final solution. Thus, in some embodiments, at the outset all expression values are standardized to have mean zero and a standard deviation of one. This ensures all inputs are treated equally in the regularization process, and allows one to choose a meaningful range for the random starting weights. With standardization inputs, it is typical to take random uniform weights over the range [−0.7, +0.7].

A recurrent problem in the use of networks having a hidden layer is the optimal number of hidden units to use in the network. The number of inputs and outputs of a network are determined by the problem to be solved. In the present invention, the number of inputs for a given neural network can be the number of molecular markers in the selected set of molecular markers of the invention. The number of output for the neural network will typically be just one. However, in some embodiment more than one output is used so that more than just two states can be defined by the network. If too many hidden units are used in a neural network, the network will have too many degrees of freedom and is trained too long, there is a danger that the network will over-fit the data. If there are too few hidden units, the training set cannot be learned. Generally speaking, however, it is better to have too many hidden units than too few. With too few hidden units, the model might not have enough flexibility to capture the nonlinearities in the data; with too many hidden units, the extra weight can be shrunk towards zero if appropriate regularization or pruning, as described below, is used. In typical embodiments, the number of hidden units is somewhere in the range of 5 to 100, with the number increasing with the number of inputs and number of training cases.

One general approach to determining the number of hidden units to use is to apply a regularization approach. In the regularization approach, a new criterion function is constructed that depends not only on the classical training error, but also on classifier complexity. Specifically, the new criterion function penalizes highly complex models; searching for the minimum in this criterion is to balance error on the training set with error on the training set plus a regularization term, which expresses constraints or desirable properties of solutions:

J+Jₚₐₜ+λJ_{reg}.

The parameter λ is adjusted to impose the regularization more or less strongly. In other words, larger values for λ will tend to shrink weights towards zero: typically cross-validation with a validation set is used to estimate λ. This validation set can be obtained by setting aside a random subset of the training population. Other forms of penalty can also be used, for example the weight elimination penalty (see, e.g., Hastie et al., 2001, The Elements of Statistical Learning, Springer-Verlag, New York).

Another approach to determine the number of hidden units to use is to eliminate—prune—weights that are least needed. In one approach, the weights with the smallest magnitude are eliminated (set to zero). Such magnitude-based pruning can work, but is non-optimal; sometimes weights with small magnitudes are important for learning and training data. In some embodiments, rather than using a magnitude-based pruning approach, Wald statistics are computed. The fundamental idea in Wald Statistics is that they can be used to estimate the importance of a hidden unit (weight) in a model. Then, hidden units having the least importance are eliminated (by setting their input and output weights to zero). Two algorithms in this regard are the Optimal Brain Damage (OBD) and the Optimal Brain Surgeon (OBS) algorithms that use second-order approximation to predict how the training error depends upon a weight, and eliminate the weight that leads to the smallest increase in training error.

Optimal Brain Damage and Optimal Brain Surgeon share the same basic approach of training a network to local minimum error at weight w, and then pruning a weight that leads to the smallest increase in the training error. The predicted functional increase in the error for a change in full weight vector δw is:

$$δ J = {( \frac{\partial J}{\partial w} )}^{t} \cdot δ w + \frac{1}{2} δ {w}^{t} \cdot \frac{{\partial }^{2} J}{\partial {w}^{2}} \cdot δ w + O ( {‖ δ w ‖}^{3} )$$

$$where \frac{{\partial }^{2} J}{\partial {w}^{2}}$$

is the Hessian matrix. The first term vanishes because we are at a local minimum in error; third and higher order terms are ignored. The general solution for minimizing this function given the constraint of deleting one weight is:

$$δ w = - \frac{{w}_{q}}{{[ {H}^{- 1} ]}_{qq}} {H}^{- 1} \cdot {u}_{q} and {L}_{q} = \frac{1}{2} - \frac{{w}_{q}^{2}}{{[ {H}^{- 1} ]}_{qq}}$$

Here, u_{q} is the unit vector along the qth direction in weight space and L_{q} is approximation to the saliency of the weight q—the increase in training error if weight q is pruned and the other weights updated δw. These equations require the inverse of H. One method to calculate this inverse matrix is to start with a small value, H₀⁻¹=α⁻¹I, where α is a small parameter—effectively a weight constant. Next the matrix is updated with each pattern according to

$${H}_{m + 1}^{- 1} = {H}_{m}^{- 1} - \frac{{H}_{m}^{- 1} {X}_{m + 1} {X}_{m + 1}^{T} {H}_{m}^{- 1}}{\frac{n}{{a}_{m}} + {X}_{m + 1}^{T} {H}_{m}^{- 1} {X}_{m + 1}}$$

where the subscripts correspond to the pattern being presented and αₘ decreases with m. After the full training set has been presented, the inverse Hessian matrix is given by H⁻¹=Hₙ⁻¹. In algorithmic form, the Optimal Brain Surgeon method is:

```
begin initialize n_{H}, w, θ
  train a reasonably large network to minimum error
  do compute H⁻¹ by Equation 1
    ${q}^{*} ← arg {min}_{q} {w}_{q}^{2} / ( {2 [ {H}^{- 1} ]}_{qq} ) ( saliency {L}_{q} )$
    $w ← w - \frac{{w}_{{q}^{*}}}{{[ {H}^{- 1} ]}_{{q}^{*} {q}^{*}}} {H}^{- 1} {e}_{{q}^{*}} ( saliency {L}_{q} )$
  until J(w) > θ
return w
end
```

The Optimal Brain Damage method is computationally simpler because the calculation of the inverse Hessian matrix in line 3 is particularly simple for a diagonal matrix. The above algorithm terminates when the error is greater than a criterion initialized to be θ. Another approach is to change line 6 to terminate when the change in J(w) due to elimination of a weight is greater than some criterion value.

In some embodiments, a back-propagation neural network (see, for example Abdi, 1994, “A neural network primer”, J. Biol System. 2, 247-283) containing a single hidden layer of ten neurons (ten hidden units) found in EasyNN-Plus version 4.0 g software package (Neural Planner Software Inc.) is used. In a specific example, parameter values within the EasyNN-Plus program are set as follows: a learning rate of 0.05, and a momentum of 0.2. In some embodiments in which the EasyNN-Plus version 4.0 g software package is used, “outlier” samples are identified by performing twenty independently-seeded trials involving 20,000 learning cycles each.

### 5.2.3. Support Vector Machine

In some embodiments of the present invention, support vector machines (SVMs) are used to classify subjects using expression profiles of marker genes described in the present invention. The SVM takes the patient marker profile as an input and generates an output comprising data indicating whether the patient is predicted to be a responsive or a non-responsive patient, e.g., a CR score. General description of SVM can be found in, for example, Cristianini and Shawe-Taylor, 2000, An Introduction to Support Vector Machines, Cambridge University Press, Cambridge, Boser et al., 1992, “A training algorithm for optimal margin classifiers, in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Duda, Pattern Classification, Second Edition, 2001, John Wiley & Sons, Inc.; Hastie, 2001, The Elements of Statistical Learning, Springer, New York; and Furey et al., 2000, Bioinformatics 16, 906-914. Applications of SVM in biological applications are described in Jaakkola et al., Proceedings of the 7^{th} International

Conference on Intelligent Systems for Molecular Biology, AAAI Press, Menlo Park, Calif. (1999); Brown et al., Proc. Natl. Acad. Sci. 97(1):262-67 (2000); Zien et al., Bioinformatics, 16(9):799-807 (2000); Furey et al., Bioinformatics, 16(10):906-914 (2000)

In one approach, when a SVM is used, the gene expression data is standardized to have mean zero and unit variance and the members of a training population are randomly divided into a training set and a test set. For example, in one embodiment, two thirds of the members of the training population are placed in the training set and one third of the members of the training population are placed in the test set. The expression values for a selected set of genes of the present invention are used to train the SVM. Then the ability for the trained SVM to correctly classify members in the test set is determined. In some embodiments, this computation is performed several times for a given selected set of molecular markers. In each iteration of computation, the members of the training population are randomly assigned to the training set and the test set. Then, the quality of the combination of molecular markers is taken as the average of each such iteration of the SVM computation.

Support vector machines map a given set of binary labeled training data to a high-dimensional feature space and separate the two classes of data with a maximum margin hyperplane. In general, this hyperplane corresponds to a nonlinear decision boundary in the input space. Let X ∈ R₀ ⊂ [symbol] ⁿ be the input vectors, y ∈ {−1,+1} be the labels, and φ: R₀→F be the mapping from input space to feature space. Then the SVM learning algorithm finds a hyperplane (w, b) such that the quantity

$$γ = {min}_{i} {y}_{i} \{ 〈 w , φ ( {X}_{i} ) 〉 - b \}$$

is maximized, where the vector w has the same dimensionality as F, b is a real number, and γ is called the margin. The corresponding decision function is then

ƒ(X)=sign( [symbol] w,φ(X) [symbol] −b)

This minimum occurs when

$$w = {\sum }_{i} {α}_{i} {y}_{i} φ ( {X}_{i} )$$

where {αᵢ} are positive real numbers that maximize

$${\sum }_{i} {α}_{i} - {\sum }_{ij} {α}_{i} {α}_{j} {y}_{i} {y}_{j} 〈 φ ( {X}_{i} ) , φ ( {X}_{j} ) 〉$$

subject to

$${\sum }_{i} {α}_{i} {y}_{i} = 0 , {α}_{i} > 0.$$

The decision function can equivalently be expressed as

$$f ( X ) = sign ( {\sum }_{i} {α}_{i} {y}_{i} 〈 φ ( {X}_{i} , φ ( X ) 〉 - b )$$

From this equation it can be seen that the αᵢ associated with the training point Xᵢ expresses the strength with which that point is embedded in the final decision function. A remarkable property of this alternative representation is that only a subset of the points will be associated with a non-zero αᵢ. These points are called support vectors and are the points that lie closest to the separating hyperplane. The sparseness of the α vector has several computational and learning theoretic consequences. It is important to note that neither the learning algorithm nor the decision function needs to represent explicitly the image of points in the feature space, φ(Xᵢ), since both use only the dot products between such images, [symbol] φ(Xᵢ),φ(Xⱼ) [symbol] . Hence, if one were given a function K(X,Y)= [symbol] φ(X),φ(X) [symbol] , one could learn and use the maximum margin hyperplane in the feature space without ever explicitly performing the mapping. For each continuous positive definite function K(X,Y) there exists a mapping φ such that K(X,Y)= [symbol] φ(X),φ(X) [symbol] for all X,Y ∈ R₀ (Mercer's Theorem). The function K(X,Y) is called the kernel function. The use of a kernel function allows the support vector machine to operate efficiently in a nonlinear high-dimensional feature spaces without being adversely affected by the dimensionality of that space. Indeed, it is possible to work with feature spaces of infinite dimension. Moreover, Mercer's theorem makes it possible to learn in the feature space without even knowing φ and F. The matrix Kᵢⱼ= [symbol] φ(Xᵢ),φ(Xⱼ) [symbol] is called the kernel matrix. Finally, note that the learning algorithm is a quadratic optimization problem that has only a global optimum. The absence of local minima is a significant difference from standard pattern recognition techniques such as neural networks. For moderate sample sizes, the optimization problem can be solved with simple gradient descent techniques. In the presence of noise, the standard maximum margin algorithm described above can be subject to over-fitting, and more sophisticated techniques should be used. This problem arises because the maximum margin algorithm always finds a perfectly consistent hypothesis and does not tolerate training error. Sometimes, however, it is necessary to trade some training accuracy for better predictive power. The need for tolerating training error has led to the development the soft-margin and the margin-distribution classifiers. One of these techniques replaces the kernel matrix in the training phase as follows:

K←K+λI

while still using the standard kernel function in the decision phase. By tuning λ, one can control the training error, and it is possible to prove that the risk of misclassifying unseen points can be decreased with a suitable choice of λ.

If instead of controlling the overall training error one wants to control the trade-off between false positives and false negatives, it is possible to modify K as follows:

K←K+λD

where D is a diagonal matrix whose entries are either d⁺ or d⁻, in locations corresponding to positive and negative examples. It is possible to prove that this technique is equivalent to controlling the size of the αᵢ in a way that depends on the size of the class, introducing a bias for larger αᵢ in the class with smaller d. This in turn corresponds to an asymmetric margin;

i.e., the class with smaller d will be kept further away from the decision boundary. In some cases, the extreme imbalance of the two classes, along with the presence of noise, creates a situation in which points from the minority class can be easily mistaken for mislabeled points. Enforcing a strong bias against training errors in the minority class provides protection against such errors and forces the SVM to make the positive examples support vectors. Thus, choosing

$${d}^{+} = \frac{1}{{n}^{+}} and {d}^{-} = \frac{1}{{n}^{-}}$$

provides a heuristic way to automatically adjust the relative importance of the two classes, based on their respective cardinalities. This technique effectively controls the trade-off between sensitivity and specificity.

In the present invention, a linear kernel can be used. The similarity between two marker profiles X and Y can be the dot product X·Y. In one embodiment, the kernel is

K(X,Y)=X·Y+1

In another embodiment, a kernel of degree d is used

K(X,Y)=(X·Y+1)^{d}, where d can be either 2, 3, . . .

In still another embodiment, a Gaussian kernel is used

$$K ( X , Y ) = exp ( \frac{- {\vert X - Y \vert}^{2}}{2 {σ}^{2}} )$$

where σ is the width of the Gaussian.

### 5.2.4. Logistic Regression

In some embodiments, the chemotherapy response classifier is based on a regression model, preferably a logistic regression model. Such a regression model includes a coefficient for each of the molecular markers in a selected set of molecular markers of the invention. In such embodiments, the coefficients for the regression model are computed using, for example, a maximum likelihood approach. In particular embodiments, molecular marker data from two different clinical groups, e.g., responsive and non-responsive, is used and the dependent variable is the clinical status of the patient for which molecular marker characteristic data are from.

Some embodiments of the present invention provide generalizations of the logistic regression model that handle multicategory (polychotomous) responses. Such embodiments can be used to discriminate an organism into one or three or more clinical groups, e.g., chronic phase, accelerated phase, and blast phase. Such regression models use multicategory logit models that simultaneously refer to all pairs of categories, and describe the odds of response in one category instead of another. Once the model specifies logits for a certain (J-1) pairs of categories, the rest are redundant. See, for example, Agresti, An Introduction to Categorical Data Analysis, John Wiley & Sons, Inc., 1996, New York, Chapter 8, which is hereby incorporated by reference.

### 5.2.5. Discriminant Analysis

Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. In the present invention, the expression values for the selected set of molecular markers of the invention across a subset of the training population serve as the requisite continuous independent variables. The clinical group classification of each of the members of the training population serves as the dichotomous categorical dependent variable.

LDA seeks the linear combination of variables that maximizes the ratio of between-group variance and within-group variance by using the grouping information. Implicitly, the linear weights used by LDA depend on how the expression of a molecular marker across the training set separates in the two groups (e.g., a responsive group and a non-responsive group) and how this gene expression correlates with the expression of other genes. In some embodiments, LDA is applied to the data matrix of the N members in the training sample by K genes in a combination of genes described in the present invention. Then, the linear discriminant of each member of the training population is plotted. Ideally, those members of the training population representing a first subgroup (e.g. responsive subjects) will cluster into one range of linear discriminant values (e.g., negative) and those member of the training population representing a second subgroup (e.g. non-responsive subjects) will cluster into a second range of linear discriminant values (e.g., positive). The LDA is considered more successful when the separation between the clusters of discriminant values is larger. For more information on linear discriminant analysis, see Duda, Pattern Classification, Second Edition, 2001, John Wiley & Sons, Inc; and Hastie, 2001, The Elements of Statistical Learning, Springer, New York; Venables & Ripley, 1997, Modern Applied Statistics with s-plus, Springer, New York.

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

### 5.2.6. Decision Trees

In some embodiments of the present invention, decision trees are used to classify patients using expression data for a selected set of molecular markers of the invention. Decision tree algorithms belong to the class of supervised learning algorithms. The aim of a decision tree is to induce a classifier (a tree) from real-world example data. This tree can be used to classify unseen examples which have not been used to derive the decision tree.

A decision tree is derived from training data. An example contains values for the different attributes and what class the example belongs. In one embodiment, the training data is expression data for a combination of genes described in the present invention across the training population.

The following algorithm describes a decision tree derivation:

```
Tree(Examples,Class,Attributes)
    Create a root node
    If all Examples have the same Class value, give the root this label
    Else if Attributes is empty label the root according to the most
    common value
    Else begin
        Calculate the information gain for each attribute
        Select the attribute A with highest information gain and make
this the root attribute
        For each possible value, v, of this attribute
            Add a new branch below the root, corresponding to A = v
            Let Examples(v) be those examples with A = v
            If Examples(v) is empty, make the new branch a leaf
node labeled with the  most common value among Examples
            Else let the new branch be the tree created by
                Tree(Examples(v),Class,Attributes - {A})
    end
```

A more detailed description of the calculation of information gain is shown in the following. If the possible classes vᵢ of the examples have probabilities P(vᵢ) then the information content I of the actual answer is given by:

$$I ( P ( {v}_{i} ) , … , P ( {v}_{n} ) ) = {\sum }_{i = 1}^{n} - P ( {v}_{i} ) {log}_{2} P ( {v}_{i} )$$

The I-value shows how much information we need in order to be able to describe the outcome of a classification for the specific dataset used. Supposing that the dataset contains p positive (e.g. responsive) and n negative (e.g. non-responsive) examples (e.g. individuals), the information contained in a correct answer is:

$$I ( \frac{p}{p + n} , \frac{n}{p + n} ) = - \frac{p}{p \div n} {log}_{2} \frac{p}{p + n} - \frac{n}{p + n} {log}_{2} \frac{n}{p + n}$$

where log₂ is the logarithm using base two. By testing single attributes the amount of information needed to make a correct classification can be reduced. The remainder for a specific attribute A (e.g. a gene) shows how much the information that is needed can be reduced.

$$Remainder ( A ) = {\sum }_{i = 1}^{v} \frac{{p}_{i} + {n}_{i}}{p + n} I ( \frac{{p}_{i}}{{p}_{i} + {n}_{i}} , \frac{{n}_{i}}{{p}_{i} \div {n}_{i}} )$$

“v” is the number of unique attribute values for attribute A in a certain dataset, “i” is a certain attribute value, “pᵢ” is the number of examples for attribute A where the classification is positive (e.g. cancer), “nᵢ” is the number of examples for attribute A where the classification is negative (e.g. healthy).

The information gain of a specific attribute A is calculated as the difference between the information content for the classes and the remainder of attribute A:

$$Gain ( A ) = I ( \frac{p}{p + n} , \frac{n}{p \div n} ) - Remainder ( A )$$

The information gain is used to evaluate how important the different attributes are for the classification (how well they split up the examples), and the attribute with the highest information.

In general there are a number of different decision tree algorithms, many of which are described in Duda, Pattern Classification, Second Edition, 2001, John Wile_{y} & Sons, Inc.

Decision tree algorithms often require consideration of feature processing, impurity measure, stopping criterion, and pruning. Specific decision tree algorithms include, cut are not limited to classification and regression trees (CART), multivariate decision trees, ID3, and C4.5.

In one approach, when an exemplary embodiment of a decision tree is used, the gene expression data for a selected set of molecular markers of the invention across a training population is standardized to have mean zero and unit variance. The members of the training population are randomly divided into a training set and a test set. For example, in one embodiment, two thirds of the members of the training population are placed in the training set and one third of the members of the training population are placed in the test set. The expression values for a select combination of genes described in the present invention is used to construct the decision tree. Then, the ability for the decision tree to correctly classify members in the test set is determined. In some embodiments, this computation is performed several times for a given combination of molecular markers. In each iteration of the computation, the members of the training population are randomly assigned to the training set and the test set. Then, the quality of the combination of molecular markers is taken as the average of each such iteration of the decision tree computation.

### 5.2.7. Clustering

In some embodiments, the expression values for a selected set of molecular markers of the invention are used to cluster a training set. For example, consider the case in which ten genes described in the present invention are used. Each member m of the training population will have expression values for each of the ten genes. Such values from a member m in the training population define the vector:

| | | | | | | | | | |
|---|---|---|---|---|---|---|---|---|---|
| X₁ₘ | X₂ₘ | X₃ₘ | X₄ₘ | X₅ₘ | X₆ₘ | X₇ₘ | X₈ₘ | X₉ₘ | X₁₀ₘ |

where Xᵢₘ is the expression level of the i^{th} gene in organism m. If there are m organisms in the training set, selection of i genes will define m vectors. Note that the methods of the present invention do not require that each the expression value of every single gene used in the vectors be represented in every single vector m. In other words, data from a subject in which one of the i^{th} genes is not found can still be used for clustering. In such instances, the missing expression value is assigned either a “zero” or some other normalized value. In some embodiments, prior to clustering, the gene expression values are normalized to have a mean value of zero and unit variance.

Those members of the training population that exhibit similar expression patterns across the training group will tend to cluster together. A particular combination of genes of the present invention is considered to be a good classifier in this aspect of the invention when the vectors cluster into the trait groups found in the training population. For instance, if the training population includes responsive patients and non-responsive patient, a clustering classifier will cluster the population into two groups, with each group uniquely representing either the responsive group or the non-responsive group.

Clustering is described on pages 211-256 of Duda and Hart, Pattern Classification and Scene Analysis, 1973, John Wiley & Sons, Inc., New York. As described in Section 6.7 of Duda, the clustering problem is described as one of finding natural groupings in a dataset.

To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda, clustering does not require the use of a distance metric. For example, a nonmetric similarity function s(x, x′) can be used to compare two vectors x and x′. Conventionally, s(x, x′) is a symmetric function whose value is large when x and x′ are somehow “similar”. An example of a nonmetric similarity function s(x, x′) is provided on page 216 of Duda.

Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda. Criterion functions are discussed in Section 6.8 of Duda.

More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

### 5.2.8. Principal Component Analysis

Principal component analysis (PCA) has been proposed to analyze gene expression data. Principal component analysis is a classical technique to reduce the dimensionality of a data set by transforming the data to a new set of variable (principal components) that summarize the features of the data. See, for example, Jolliffe, 1986, Principal Component Analysis, Springer, N.Y. Principal components (PCs) are uncorrelate and are ordered such that the k^{th} PC has the kth largest variance among PCs. The k^{th} PC can be interpreted as the direction that maximizes the variation of the projections of the data points such that it is orthogonal to the first k-1 PCs. The first few PCs capture most of the variation in the data set. In contrast, the last few PCs are often assumed to capture only the residual ‘noise’ in the data.

PCA can also be used to create a chemotherapy response classifier in accordance with the present invention. In such an approach, vectors for a selected set of molecular markers of the invention can be constructed in the same manner described for clustering above. In fact, the set of vectors, where each vector represents the expression values for the select genes from a particular member of the training population, can be considered a matrix. In some embodiments, this matrix is represented in a Free-Wilson method of qualitative binary description of monomers (Kubinyi, 1990, 3D QSAR in drug design theory methods and applications, Pergamon Press, Oxford, pp 589-638), and distributed in a maximally compressed space using PCA so that the first principal component (PC) captures the largest amount of variance information possible, the second principal component (PC) captures the second largest amount of all variance information, and so forth until all variance information in the matrix has been accounted for.

Then, each of the vectors (where each vector represents a member of the training population) is plotted. Many different types of plots are possible. In some embodiments, a one-dimensional plot is made. In this one-dimensional plot, the value for the first principal component from each of the members of the training population is plotted. In this form of plot, the expectation is that members of a first group (e.g. chronic phase patients) will cluster in one range of first principal component values and members of a second group (e.g., advance phase patients) will cluster in a second range of first principal component values.

In one example, the training population comprises two groups: a responder group and a non-responder group. The first principal component is computed using the molecular marker expression values for the select genes of the present invention across the entire training population data set. Then, each member of the training set is plotted as a function of the value for the first principal component. In this example, those members of the training population in which the first principal component is positive are the responders and those members of the training population in which the first principal component is negative are the non-responders.

In some embodiments, the members of the training population are plotted against more than one principal component. For example, in some embodiments, the members of the training population are plotted on a two-dimensional plot in which the first dimension is the first principal component and the second dimension is the second principal component. In such a two-dimensional plot, the expectation is that members of each subgroup represented in the training population will cluster into discrete groups. For example, a first cluster of members in the two-dimensional plot will represent responsive subjects, a second cluster of members in the two-dimensional plot will represent non-responsive subjects, and so forth.

In some embodiments, the members of the training population are plotted against more than two principal components and a determination is made as to whether the members of the training population are clustering into groups that each uniquely represents a subgroup found in the training population. In some embodiments, principal component analysis is performed by using the R mva package (Anderson, 1973, Cluster Analysis for applications, Academic Press, New York 1973; Gordon, Classification, Second Edition, Chapman and Hall, CRC, 1999.). Principal component analysis is further described in Duda, Pattern Classification, Second Edition, 2001, John Wiley & Sons, Inc.

### 5.2.9. Nearest Neighbor Classifier Analysis

Nearest neighbor classifiers are memory-based and require no model to be fit. Given a query point x₀, the k training points x₍ᵣ₎, r, . . . , k closest in distance to x₀ are identified and then the point x₀ is classified using the k nearest neighbors. Ties can be broken at random. In some embodiments, Euclidean distance in feature space is used to determine distance as:

d₍ᵢ₎=∥x₍ᵢ₎−xₒₗ∥.

Typically, when the nearest neighbor algorithm is used, the expression data used to compute the linear discriminant is standardized to have mean zero and variance 1. In the present invention, the members of the training population are randomly divided into a training set and a test set. For example, in one embodiment, two thirds of the members of the training population are placed in the training set and one third of the members of the training population are placed in the test set. Profiles of a selected set of molecular markers of the invention represent the feature space into which members of the test set are plotted. Next, the ability of the training set to correctly characterize the members of the test set is computed. In some embodiments, nearest neighbor computation is performed several times for a given combination of genes of the present invention. In each iteration of computation, the members of the training population are randomly assigned to the training set and the test set. Then, the quality of the combination of genes is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, N.Y.

### 5.2.10. Evolutionary Methods

Inspired by the process of biological evolution, evolutionary methods of classifier design employ a stochastic search for an optimal classifier. In broad overview, such methods create several classifiers—a population—from measurements of gene products of the present invention. Each classifier varies somewhat from the other. Next, the classifiers are scored on expression data across the training population. In keeping with the analogy with biological evolution, the resulting (scalar) score is sometimes called the fitness. The classifiers are ranked according to their score and the best classifiers are retained (some portion of the total population of classifiers). Again, in keeping with biological terminology, this is called survival of the fittest. The classifiers are stochastically altered in the next generation—the children or offspring. Some offspring classifiers will have higher scores than their parent in the previous generation, some will have lower scores. The overall process is then repeated for the subsequent generation: The classifiers are scored and the best ones are retained, randomly altered to give yet another generation, and so on. In part, because of the ranking, each generation has, on average, a slightly higher score than the previous one. The process is halted when the single best classifier in a generation has a score that exceeds a desired criterion value. More information on evolutionary methods is found in, for example, Duda, Pattern Classification, Second Edition, 2001, John Wiley & Sons, Inc.

### 5.2.11. Bagging, Boosting and the Random Subspace Method

Bagging, boosting and the random subspace method are combining techniques that can be used to improve weak classifiers. These techniques are designed for, and usually applied to, decision trees. In addition, Skurichina and Duin provide evidence to suggest that such techniques can also be useful in linear discriminant analysis.

In bagging, one samples the training set, generating random independent bootstrap replicates, constructs the classifier on each of these, and aggregates them by a simple majority vote in the final decision rule. See, for example, Breiman, 1996, Machine Learning 24, 123-140; and Efron & Tibshirani, An Introduction to Bootstrap, Chapman & Hall, New York, 1993.

In boosting, classifiers are constructed on weighted versions of the training set, which are dependent on previous classification results. Initially, all objects have equal weights, and the first classifier is constructed on this data set. Then, weights are changed according to the performance of the classifier. Erroneously classified objects (molecular markers in the data set) get larger weights, and the next classifier is boosted on the reweighted training set. In this way, a sequence of training sets and classifiers is obtained, which is then combined by simple majority voting or by weighted majority voting in the final decision. See, for example, Freund & Schapire, “Experiments with a new boosting algorithm,” Proceedings 13^{th} International Conference on Machine Learning, 1996, 148-156.

To illustrate boosting, consider the case where there are two phenotypic groups exhibited by the population under study, phenotype 1 (e.g., advanced phase patients), and phenotype 2 (e.g., chronic phase patients). Given a vector of molecular markers X, a classifier G(X) produces a prediction taking one of the type values in the two value set: {phenotype 1, phenotype 2}. The error rate on the training sample is

$$\overline{err} = \frac{1}{N} {\sum }_{i = 1}^{N} I ( {y}_{i} \neq G ( {x}_{i} ) )$$

where N is the number of subjects in the training set (the sum total of the subjects that have either phenotype 1 or phenotype 2).

A weak classifier is one whose error rate is only slightly better than random guessing. In the boosting algorithm, the weak classification algorithm is repeatedly applied to modified versions of the data, thereby producing a sequence of weak classifiers Gₘ(x), m,=1, 2, . . . , M. The predictions from all of the classifiers in this sequence are then combined through a weighted majority vote to produce the final prediction:

$$G ( x ) = sign ( {\sum }_{m = 1}^{M} {α}_{m} {G}_{m} ( x ) )$$

Here α₁, α₂, . . . , α_{M} are computed by the boosting algorithm and their purpose is to weigh the contribution of each respective Gₘ(x). Their effect is to give higher influence to the more accurate classifiers in the sequence.

The data modifications at each boosting step consist of applying weights w₁, w₂, . . . , wₙ to each of the training observations (xᵢ, yᵢ), i=1, 2, . . . , N. Initially all the weights are set to wᵢ=1/N, so that the first step simply trains the classifier on the data in the usual manner. For each successive iteration m=2, 3, . . . , M the observation weights are individually modified and the classification algorithm is reapplied to the weighted observations. At stem m, those observations that were misclassified by the classifier Gₘ₋₁(x) induced at the previous step have their weights increased, whereas the weights are decreased for those that were classified correctly. Thus as iterations proceed, observations that are difficult to correctly classify receive ever-increasing influence. Each successive classifier is thereby forced to concentrate on those training observations that are missed by previous ones in the sequence.

The exemplary boosting algorithm is summarized as follows:

1. Initialize the observation weights wᵢ=1/N, i=1, 2, . . . , N.

2. For m=1 to M:
(a) Fit a classifier Gₘ(x) to the training set using weights wᵢ.
(b) Compute

$${err}_{m} = \frac{{\sum }_{i = 1}^{N} {w}_{i} I ( {y}_{i} \neq {G}_{m} ( {x}_{i} ) )}{{\sum }_{i = 1}^{N} {w}_{i}}$$

(c) Compute αₘ=log((1-errₘ)/errₘ).

(d) Set wᵢ←wᵢ·exp[αₘ·(yᵢ≠Gₘ(xᵢ))],i=1, 2, . . . , N.

$$3. Output G ( x ) = sign \vert {\sum }_{m = 1}^{M} {α}_{m} {G}_{m} ( x ) \vert \qquad$$

In the algorithm, the current classifier Gₘ(x) is induced on the weighted observations at line 2a. The resulting weighted error rate is computed at line 2b. Line 2c calculates the weight αₘ given to Gₘ(x) in producing the final classifier G(x) (line 3). The individual weights of each of the observations are updated for the next iteration at line 2d. Observations misclassified by Gₘ(x) have their weights scaled by a factor exp(αₘ), increasing their relative influence for inducing the next classifier Gₘ₊₁(x) in the sequence. In some embodiments, modifications of the Freund and Schapire, 1997, Journal of Computer and System Sciences 55, pp. 119-139, boosting method are used. See, for example, Hasti et al., The Elements of Statistical Learning, 2001, Springer, N.Y., Chapter 10. In some embodiments, boosting or adaptive boosting methods are used.

In some embodiments, modifications of Freund and Schapire, 1997, Journal of Computer and System Sciences 55, pp. 119-139, are used. For example, in some embodiments, feature preselection is performed using a technique such as the nonparametric scoring methods of Park et al., 2002, Pac. Symp. Biocomput. 6, 52-63. Feature preselection is a form of dimensionality reduction in which the genes that discriminate between classifications the best are selected for use in the classifier. Then, the LogitBoost procedure introduced by Friedman et al., 2000, Ann Stat 28, 337-407 is used rather than the boosting procedure of Freund and Schapire. In some embodiments, the boosting and other classification methods of Ben-Dor et al., 2000, Journal of Computational Biology 7, 559-583 are used in the present invention. In some embodiments, the boosting and other classification methods of Freund and Schapire, 1997, Journal of Computer and System Sciences 55, 119-139, are used.

In the random subspace method, classifiers are constructed in random subspaces of the data feature space. These classifiers are usually combined by simple majority voting in the final decision rule. See, for example, Ho, “The Random subspace method for constructing decision forests,” IEEE Trans Pattern Analysis and Machine Intelligence, 1998; 20(8): 832-844.

### 5.2.12. Other Algorithms

The pattern classification and statistical techniques described above are merely examples of the types of models that can be used to construct a model for classification. Moreover, combinations of the techniques described above can be used. Some combinations, such as the use of the combination of decision trees and boosting, have been described. However, many other combinations are possible. In addition, in other techniques in the art such as Projection Pursuit and Weighted Voting can be used to construct a chemotherapy response classifier.

### 5.3. Methods of Determining Expression Levels of Chemotherapy Response Genes

The invention also provides methods and compositions for determining expression levels of CR genes, i.e., marker genes listed in Table 1 and/or their encoded proteins. Such information can be used to determine a treatment regimen for a patient. For example, a patient whose level of expression of one or more CR genes predicts that the patient is responsive to a chemotherapeutic agent can be assigned a treatment regimen comprising the chemotherapeutic agent. A patient whose level of expression of one or more CR genes predicts that the patient is non-responsive to a chemotherapeutic agent can either be assigned a treatment regimen that does not comprise the chemotherapeutic agent, or assigned a treatment regimen including a combination of the chemotherapeutic agent and a therapy to regulate the expression levels of the gene or genes. Thus, the invention provides methods and composition for assigning treatment regimen for a cancer patient. The invention also provides methods and composition for monitoring treatment progress for a cancer patient based on the expression levels of the marker genes.

A variety of methods can be employed for the diagnostic and prognostic evaluation of patients for their responsiveness to chemotherapy. In one embodiment, measurements of expression level of one or more of CR genes listed in Table 1, and/or abundance or activity level the encoded proteins are used.

In one embodiment, the method comprises determining an expression level of a chemotherapy response gene listed in Table 1 in a sample of a patient, and determining whether the expression level is deviated (above or below) from a predetermined threshold that separates responsive and non-responsive patients. In another embodiment, the method comprises determining a level of abundance of a protein encoded by a CR gene, in a sample from a patient, and determining whether the level of abundance is deviated from a predetermined threshold that separates responsive and non-responsive patients. In still another embodiment, the method comprises determining a level of activity of a protein encoded by a CR gene in a sample of a patient, and determining whether the level of activity is deviated from a predetermined threshold that separates responsive and non-responsive patients. In the foregoing embodiments, and the embodiments described below, the sample can be an ex vivo cell sample, e.g., cells in a cell culture, or in vivo cells.

In a specific embodiment, the method comprises determining an expression level of an interferon stimulated gene (ISG) listed in Table 1 in the sample of a patient, and determining whether the expression level is above a predetermined threshold. In another embodiment, the method comprises determining a level of abundance of a protein encoded by an ISG gene, and determining whether the level is above a predetermined threshold.

Such methods may, for example, utilize reagents such as nucleotide sequences and antibodies, e.g., the chemotherapy response nucleotide sequences, and antibodies directed against chemotherapy response proteins, including peptide fragments thereof. Specifically, such reagents may be used, for example, for: (1) the detection of the presence of mutations in a chemotherapy response gene, or the detection of either over- or under-expression of a chemotherapy response gene relative to the normal expression level; and (2) the detection of either an over- or an under-abundance of a chemotherapy response protein relative to the threshold chemotherapy response protein level.

The methods described herein may be performed, for example, by utilizing pre-packaged diagnostic kits comprising nucleic acid of at least one specific chemotherapy response gene or an antibody that binds a chemotherapy response protein, which may be conveniently used, e.g., in clinical settings, to diagnose patients exhibiting responsiveness or non-responsiveness to chemotherapy.

Nucleic acid-based detection techniques and peptide detection techniques are described in Sections 5.3.2., infra. In one embodiment, the expression levels of one or more marker genes are measured using qRT-PCR.

### 5.3.1. Samples Collection

In the present invention, gene products, such as target polynucleotide molecules or proteins, are extracted from a sample taken from a cancer patient. The sample may be collected in any clinically acceptable mariner, but must be collected such that marker-derived polynucleotides (i.e., RNA) are preserved (if gene expression is to be measured) or proteins are preserved (if encoded proteins are to be measured). In one embodiment, tumor samples are used. In one embodiment, the pre-treatment tumor sample from a patient is used. In another embodiment, the tumor sample from a patient after and/or during treatment is used. In one embodiment, the unsorted tumor sample from a patient is used. In another embodiment, the sorted tumor sample from a patient is used. Other suitable samples may comprise any clinically relevant tissue sample, such as a tumor biopsy or fine needle aspirate, or a sample of body fluid, such as blood, plasma, serum, lymph, ascitic fluid, cystic fluid, or urine. The sample may be taken from a human, or, in a veterinary context, from non-human animals such as ruminants, horses, swine or sheep, or from domestic companion animals such as felines and canines.

In a specific embodiment, mRNA or nucleic acids derived therefrom (i.e., cDNA or amplified RNA or amplified DNA) are preferably labeled distinguishably from polynucleotide molecules of a reference sample, and both are simultaneously or independently hybridized to a microarray comprising some or all of the markers or marker sets or subsets described above. Alternatively, mRNA or nucleic acids derived therefrom may be labeled with the same label as the reference polynucleotide molecules, wherein the intensity of hybridization of each at a particular probe is compared.

Methods for preparing total and poly(A)+ RNA are well known and are described generally in Sambrook et al., MOLECULAR CLONING—A LABORATORY MANUAL (2ND ED.), Vols. 1-3, Cold Spring Harbor Laboratory, Cold Spring Harbor, N.Y. (1989)) and Ausubel et al., CURRENT PROTOCOLS IN MOLECULAR BIOLOGY, vol. 2, Current Protocols Publishing, New York (1994)). Preferably, total RNA, or total mRNA (poly(A)+ RNA) is measured in the methods of the invention directly or indirectly (e.g., via measuring cDNA or cRNA).

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells. Preferably, the cells are breast cancer tumor cells.

Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCI centrifugation to separate the RNA from DNA (Chirgwin et al., Biochemistry 18:5294-5299 (1979)). Poly(A)+ RNA is selected by selection with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol.

If desired, RNase inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex™ (see Ausubel et al., CURRENT PROTOCOLS IN MOLECULAR BIOLOGY, vol. 2, Current Protocols Publishing, New York (1994). Once bound, poly(A)+ mRNA is eluted from the affinity column using 2 mM EDTA/0.1% SDS.

In a specific embodiment, total RNA or total mRNA from cells is used in the methods of the invention. The source of the RNA can be cells of an animal, e.g., human, mammal, primate, non-human animal, dog, cat, mouse, rat, bird, etc. In specific embodiments, the method of the invention is used with a sample containing total mRNA or total RNA from 1×10⁶ cells or less. In another embodiment, proteins can be isolated from the foregoing sources, by methods known in the art, for use in expression analysis at the protein level.

Probes to the homologs of the marker sequences disclosed herein can be employed preferably when non-human nucleic acid is being assayed.

### 5.3.2. Determination of Abundance Le3vels of Gene Products

The abundance levels of the gene products of the genes in a sample may be determined by any means known in the art. The levels may be determined by isolating and determining the level (i.e., amount) of nucleic acid transcribed from each marker gene. Alternatively, or additionally, the level of specific proteins encoded by a marker gene may be determined.

The levels of transcripts of specific marker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label.

The levels of transcripts of particular marker genes may also be assessed by determining the level of the specific protein expressed from the marker genes. This can be accomplished, for example, by separation of proteins from a sample on a polyacrylamide gel, followed by identification of specific marker-derived proteins using antibodies in a western blot. Alternatively, proteins can be separated by two-dimensional gel electrophoresis systems. Two-dimensional gel electrophoresis is well-known in the art and typically involves isoelectric focusing along a first dimension followed by SDS-PAGE electrophoresis along a second dimension. See, e.g., Hames et al, 1990, GEL ELECTROPHORESIS OF PROTEINS: A PRACTICAL APPROACH, IRL Press, New York; Shevchenko et al., Proc. Nat'l Acad. Sci. USA 93:1440-1445 (1996); Sagliocco et al., Yeast 12:1519-1533 (1996); Lander, Science 274:536-539 (1996). The resulting electropherograms can be analyzed by numerous techniques, including mass spectrometric techniques, western blotting and immunoblot analysis using polyclonal and monoclonal antibodies.

Alternatively, marker-derived protein levels can be determined by constructing an antibody microarray in which binding sites comprise immobilized, preferably monoclonal, antibodies specific to a plurality of protein species encoded by the cell genome. Preferably, antibodies are present for a substantial fraction of the marker-derived proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

### 5.3.2.1. Microarrays

In preferred embodiments, polynucleotide microarrays are used to measure expression so that the expression status of each of the markers above is assessed simultaneously.

Generally, microarrays according to the invention comprise a plurality of markers informative for clinical category determination, for a particular disease or condition.

The invention also provides a microarray comprising for each of one or more genes listed in Table 1, one or more polynucleotide probes complementary and hybridizable to a sequence in said gene, wherein polynucleotide probes complementary and hybridizable to said genes constitute at least X% of the probes on said microarray, X %=50%, 60%, 70%, 80%, 90%, 95%, or 98%. In a particular embodiment, the invention provides such a microarray wherein the one or more genes comprises all genes listed in Table 1. The microarray can be in a sealed container.

The microarrays preferably comprise at least N, where N=2, 3, 4, 5, 7, 10, 15, 20, 25, 30, or 35, or all of the markers, or any combination of markers listed in Table 1. The actual number of informative markers the microarray comprises will vary depending upon the particular condition of interest.

In other embodiments, the invention provides polynucleotide arrays in which the chemotherapy response markers comprise at least X% of the probes on the array, where X %=50%, 60%, 70%, 80%, 85%, 90%, 95% or 98%. In another specific embodiment, the microarray comprises a plurality of probes, wherein said plurality of probes comprise probes complementary and hybridizable to at least 75% of the chemotherapy response markers.

General methods pertaining to the construction of microarrays comprising the marker sets and/or subsets above are described in the following sections.

### 5.3.2.2. Construction of Microarrays

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In preferred embodiments, a microarray comprises a support or surface with an ordered array of binding (e.g., hybridization) sites or “probes” each representing one of the markers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to the invention, the microarray is an array (i.e., a matrix) in which each position represents one of the markers described herein. For example, each position can contain a DNA or DNA analogue based on genomic DNA to which a particular RNA or cDNA transcribed from that genetic marker can specifically hybridize. The DNA or DNA analogue can be, e.g., a synthetic oligomer or a gene fragment. In one embodiment, probes representing each of the markers are present on the array. In a preferred embodiment, the array comprises probes for each of the markers listed in Table 1.

### 5.3.2.3. Preparing Probes for Microarrays

As noted above, the “probe” to which a particular polynucleotide molecule specifically hybridizes according to the invention contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR PROTOCOLS: A GUIDE TO METHODS AND APPLICATIONS, Academic Press Inc., San Diego, Calif. (1990). It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure. See Friend et al., International Patent Publication WO 01/05935, published Jan. 25, 2001; Hughes et al., Nat. Biotech. 19:342-7 (2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

### 5.3.2.4. Attaching Probes to the Solid Surface

The probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6 :639-645 (1996); and Schena et al., Proc. Natl. Acad. Sci. U.S.A. 93:10539-11286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING—A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller.

In one embodiment, the arrays of the present invention are prepared by synthesizing polynucleotide probes on a support. In such an embodiment, polynucleotide probes are attached to the support covalently at either the 3′ or the 5′ end of the polynucleotide.

In a particularly preferred embodiment, microarrays are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in Synthetic DNA Arrays in Genetic Engineering, Vol. 20, J.K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm². The polynucleotide probes are attached to the support covalently at either the 3′ or the 5′ end of the polynucleotide.

### 5.3.2.5. Target Labeling and Hybridization to Microarrays

The polynucleotide molecules which may be analyzed by the present invention (the “target polynucleotide molecules”) may be from any clinically relevant source, but are expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)⁺ messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)⁺ RNA are well known in the art, and are described generally, e.g., in Sambrook et al., MOLECULAR CLONING—A LABORATORY MANUAL (2ND ED.), Vols. 1-3, Cold Spring Harbor Laboratory, Cold Spring Harbor, N.Y. (1989). In one embodiment, RNA is extracted from cells of the various types of interest in this invention using guanidinium thiocyanate lysis followed by CsC1 centrifugation (Chirgwin et al., 1979, Biochemistry 18:5294-5299). In another embodiment, total RNA is extracted using a silica gel-based column, commercially available examples of which include RNeasy (Qiagen, Valencia, Calif.) and StrataPrep (Stratagene, La Jolla, Calif.). In an alternative embodiment, which is preferred for S. cerevisiae, RNA is extracted from cells using phenol and chloroform, as described in Ausubel et al., eds., 1989, CURRENT PROTOCOLS IN MOLECULAR BIOLOGY, Vol. III, Green Publishing Associates, Inc., John Wiley & Sons, Inc., New York, at pp. 13.12.1-13.12.5). Poly(A)⁺ RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl₂, to generate fragments of RNA. In another embodiment, the polynucleotide molecules analyzed by the invention comprise cDNA, or PCR products of amplified RNA or cDNA.

In one embodiment, total RNA, mRNA, or nucleic acids derived therefrom, is isolated from a sample taken from a cancer patient. Target polynucleotide molecules that are poorly expressed in particular cells may be enriched using normalization techniques (Bonaldo et al., 1996, Genome Res. 6:791-806).

As described above, the target polynucleotides are detectably labeled at one or more nucleotides. Any method known in the art may be used to detectably label the target polynucleotides. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in a preferred embodiment, random primers (e.g., 9-mers) are used in reverse transcription to uniformly incorporate labeled nucleotides over the full length of the target polynucleotides. Alternatively, random primers may be used in conjunction with PCR methods or T7 promoter-based in vitro transcription methods in order to amplify the target polynucleotides.

In a preferred embodiment, the detectable label is a luminescent label. For example, fluorescent labels, bioluminescent labels, chemiluminescent labels, and colorimetric labels may be used in the present invention. In a highly preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a reference sample. The reference can comprise target polynucleotide molecules from normal cell samples (i. e., cell sample, e.g., of cells not afflicted with cancer) or from cell samples, e.g., tumor cells from cancer patients.

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, HYBRIDIZATION WITH NUCLEIC ACID PROBES, Elsevier Science Publishers B. V.; and Kricka, 1992, NONISOTOPIC DNA PROBE TECHNIQUES, Academic Press, San Diego, Calif.

Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

### 5.3.2.6. Signal Detection and Data Analysis

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In a preferred embodiment, the arrays are scanned with a laser fluorescent scanner with a computer controlled X-Y stage and a microscope objective. ’ Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

### 5.3.2.7. Other Assays for Detecting and Quantifying RNA

In addition to microarrays such as those described above any technique known to one of skill for detecting and measuring RNA can be used in accordance with the methods of the invention. Non-limiting examples of techniques include Northern blotting, nuclease protection assays, RNA fingerprinting, polymerase chain reaction, ligase chain reaction, Qbeta replicase, isothermal amplification method, strand displacement amplification, transcription based amplification systems, nuclease protection (SI nuclease or RNAse protection assays), SAGE as well as methods disclosed in International Publication Nos. WO 88/10315 and WO 89/06700, and International Applications Nos. PCT/US87/00880 and PCT/US89/01025.

A standard Northern blot assay can be used to ascertain an RNA transcript size, identify alternatively spliced RNA transcripts, and the relative amounts of mRNA in a sample, in accordance with conventional Northern hybridization techniques known to those persons of ordinary skill in the art. In Northern blots, RNA samples are first separated by size via electrophoresis in an agarose gel under denaturing conditions. The RNA is then transferred to a membrane, cross-linked and hybridized with a labeled probe. Nonisotopic or high specific activity radio-labeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes. The labeled probe, e.g., a radio-labeled cDNA, either containing the full-length, single stranded DNA or a fragment of that DNA sequence may be at least 20, at least 30, at least 50, or at least 100 consecutive nucleotides in length. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. Non-limiting examples of isotopes include ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

Nuclease protection assays (including both ribonuclease protection assays and Si nuclease assays) can be used to detect and quantify specific mRNAs. In nuclease protection assays, an antisense probe (labeled with, e.g., radio-labeled or nonisotopic) hybridizes in solution to an RNA sample. Following hybridiiation, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations.

The ribonuclease protection assay, which is the most common type of nuclease protection assay, requires the use of RNA probes. Oligonucleotides and other single-stranded DNA probes can only be used in assays containing S1 nuclease. The single-stranded, antisense probe must typically be completely homologous to target RNA to prevent cleavage of the probe:target hybrid by nuclease.

Serial Analysis Gene Expression (SAGE), which is described in e.g., Velculescu et al., 1995, Science 270:484-7; Carulli, et al., 1998, Journal of Cellular Biochemistry Supplements 30/31:286-96, can also be used to determine RNA abundances in a cell sample.

Quantitative reverse transcriptase PCR (qRT-PCR) can also be used to determine the expression profiles of marker genes (see, e.g., U.S. Patent Application Publication No. 2005/0048542A1). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™. Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM ⁷⁷⁰⁰™ Sequence Detection System™. The system consists of a thermocycler, laser, charge-coupled device (CCD), camera and computer. The system includes software for running the instrument and for analyzing the data.

5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

### 5.3.2.8. Detection and Quantification of Protein

Measurement of the translational state may be performed according to several methods. For example, whole genome monitoring of protein (e.g., the “proteome,”) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to the action of a drug of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array and their binding is assayed with assays known in the art.

Immunoassays known to one of skill in the art can be used to detect and quantify protein levels. For example, ELISAs can be used to detect and quantify protein levels. ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. In a preferred embodiment, an ELISA may be performed by coating a high binding 96-well microtiter plate (Costar) with 2 μg/ml of rhu-IL-9 in PBS overnight. Following three washes with PBS, the plate is incubated with three-fold serial dilutions of Fab at 25° C. for 1 hour. Following another three washes of PBS, 1 μg/ml anti-human kappa-alkaline phosphatase-conjugate is added and the plate is incubated for 1 hour at 25° C. Following three washes with PBST, the alkaline phosphatase activity is determined in 50 μl/AMP/PPMP substrate. The reactions are stopped and the absorbance at 560 nm is determined with a VMAX microplate reader. For further discussion regarding ELISAs see, e.g., Ausubel et al, eds, 1994, Current Protocols in Molecular Biology, Vol. 1, John Wiley & Sons, Inc., New York at 11.2.1.

Protein levels may be determined by Western blot analysis. Further, protein levels as well as the phosphorylation of proteins can be determined by immunoprecitation followed by Western blot analysis. Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G sepharose beads to the cell lysate, incubating for about an hour or more at 40 ° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%- 20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

Protein expression levels can also be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Natl. Acad. Sci. USA 93:1440-1445; Sagliocco et al., 1996, Yeast 12:1519-1533; Lander, 1996, Science 274:536-539. The resulting electropherograms can be analyzed by numerous techniques, including mass spectrometric techniques, Western blotting and immunoblot analysis using polyclonal and monoclonal antibodies, and internal and N-terminal micro-sequencing.

### 5.4. Treating Cancer by Modulating Expression and/or Activity of Chemotherapy Response Genes and/or their Products

The invention provides methods and compositions for utilizing chemotherapy response genes listed in Table 1 in treating cancer. The methods and compositions are used for treating non-responsive cancer patient by modulating the expression and/or activity of such genes and/or the encoded proteins in combination with a chemotherapy. The compositions (e.g., agents that modulate expression and/or activity of the CR gene or gene product) of the invention are preferably purified.

In one embodiment, the invention provides methods and compositions for treating a non-responsive cancer patient by reducing the expression and/or activity of one or more genes listed in Table 1, and/or its encoded protein by at least 2 fold, 3 fold, 4 fold, 6 fold, 8 fold or 9 fold.

In a specific embodiment, the invention provides a method for treating a non-responsive cancer patient by administering to a patient (i) an agent that is capable of reducing the expression and/or activity of one or more genes listed in Table 1, and/or its encoded protein, and (ii) a therapeutically sufficient amount of a chemotherapeutic agent. The invention also provide (i) an agent that is capable of reducing the expression and/or activity of one or more genes listed in Table 1, and/or its encoded protein, and (ii) a therapeutically sufficient amount of a chemotherapeutic agent for simultaneous or sequential use in treatment of a cancer patient, e.g., a non-responsive cancer patient. The invention also provides (i) an agent that is capable of reducing the expression and/or activity of one or more genes listed in Table 1, and/or its encoded protein, and (ii) a therapeutically sufficient amount of a chemotherapeutic agent for use in the manufacture of a medicament for simultaneous or sequential use in treatment of a cancer patient, e.g., a non-responsive cancer patient.

The invention also provides methods and compositions for utilizing chemotherapy response genes listed in Table 1 for modulating sensitivity of a cell to a chemotherapeutic drug. In one embodiment, the invention provides a method for modulating sensitivity of a cell to a chemotherapeutic drug by contacting the cell with one or more agents that are capable of reducing the expression and/or activity of one or more different genes listed in Table 1 or respective functional equivalents thereof and/or the their encoded proteins. In one embodiment, the cell is an in vivo cell. In another embodiment, the cell is an in vitro cell, e.g., a cell in a cell culture.

Thus, the invention also provides methods and compositions for modulating growth of a cell, e.g., an in vivo cell or an in vitro cell, e.g., a cell in a cell culture. In one embodiment, the invention provides a method for modulating growth of a cell, comprising contacting the cell with (a) one or more agents that are capable of reducing the expression and/or activity of one or more different genes listed in Table 1 or respective functional equivalents thereof and/or the their encoded proteins; and (b) a sufficient amount of a chemotherapeutic drug.

A variety of approaches may be used in accordance with the invention to modulate expression of a CR gene and/or its encoded protein in vivo. For example, siRNA molecules may be engineered and used to silence a CR gene in vivo. Antisense DNA molecules may also be engineered and used to block translation of a CR mRNA in vivo. Alternatively, ribozyme molecules may be designed to cleave and destroy the mRNAs of a CR gene in vivo. In another alternative, oligonucleotides designed to hybridize to the 5′ region of the CR gene (including the region upstream of the coding sequence) and form triple helix structures may be used to block or reduce transcription of the CR gene. The expression and/or activity of a CR protein can be modulated using antibody, peptide or polypeptide molecules, and small organic or inorganic molecules.

In a preferred embodiment, RNAi is used to knock down the expression of a CR gene. In one embodiment, double-stranded RNA molecules of 21-23 nucleotides which hybridize to a homologous region of mRNAs transcribed from the CR gene are used to degrade the mRNAs, thereby “silence” the expression of the CR gene. The method can be used to reduce expression levels of aberrantly up-regulated CR genes. Preferably, the dsRNAs have a hybridizing region, e.g., a 19-nucleotide double-stranded region, which is complementary to a sequence of the coding sequence of the CR gene. Any siRNA that targets an appropriate coding sequence of a CR gene and exhibit a sufficient level of silencing can be used in the invention. As exemplary embodiments, 21-nucleotide double-stranded siRNAs targeting the coding regions of a CR gene are designed according to selection rules known in the art (see, e.g., Elbashir et al., 2002, Methods 26:199-213; International Application No. PCT/US04/35636, filed Oct. 27, 2004, each of which is incorporated herein by reference in its entirety). In a preferred embodiment, the siRNA or siRNAs specifically inhibit the translation or transcription of a CR protein without substantially affecting the translation or transcription of genes encoding other protein kinases in the same kinase family. In a specific embodiment, siRNAs targeting an up-regulated gene listed in Table 4 are used to silence the respective CR genes.

The invention also provides methods and compositions for treating a non-responsive cancer patient by reducing the expression and/or activities of one or more CR genes, and/or their encoded proteins. In one embodiment, a non-responsive cancer patient is treated by administering to the patient one or more agents that reduce the expression and/or activities of these CR genes, and/or their encoded proteins. In a preferred embodiment, an siRNA is used to silence the plurality of different CR genes. The sequence of the siRNA is chosen such that the transcript of each of the genes comprises a nucleotide sequence that is identical to a central contiguous nucleotide sequence of at least 11 nucleotides of the sense strand or the antisense strand of the siRNA, and/or comprises a nucleotide sequence that is identical to a contiguous nucleotide sequence of at least 8 nucleotides at the 3′ end of the sense strand or the antisense strand of the siRNA. Thus, when administrated to the patient, the siRNA silences all of the plurality of genes in cells of the patient. In preferred embodiments, the central contiguous nucleotide sequence of the siRNA that is identical to one or more CR genes is 11-15, 14-15, 11, 12, or 13 nucleotides in length. In other preferred embodiments, the 3′ contiguous nucleotide sequence of the siRNA that is identical to one or more CR genes is 9-15, 9-12, 11, 10, 9, or 8 nucleotides in length. The length and nucleotide base sequence of the target sequence of each different target gene, i.e., the sequence of the gene that is identical to an appropriate sense or antisense sequence of the siRNA, can be different from gene to gene. For example, gene A may have a sequence of 11 nucleotides identical to the nucleotide sequence 3-13 of the sense strand of the siRNA, while gene B may have a sequence of 12 nucleotides identical to the nucleotide sequence 4-15 of the sense strand of the siRNA. Thus, a single siRNA may be designed to silence a large number of, e.g., at least 2, 3, 4, 5, 10, 15, 20, 25, 30, 35 or 39, CR genes in cells.

RNAi can be carried out using any standard method for introducing nucleic acids into cells. In one embodiment, gene silencing is induced by presenting the cell with one or more siRNAs targeting the CR gene (see, e.g., Elbashir et al., 2001, Nature 411, 494-498; Elbashir et al., 2001, Genes Dev. 15, 188-200, all of which are incorporated by reference herein in their entirety). The siRNAs can be chemically synthesized, or derived from cleavage of double-stranded RNA by recombinant Dicer. Another method to introduce a double stranded DNA (dsRNA) for silencing of the CR gene is shRNA, for short hairpin RNA (see, e.g., Paddison et al., 2002, Genes Dev. 16, 948-958; Brummelkamp et al., 2002, Science 296, 550-553; Sui, G. et al. 2002, Proc. Natl. Acad. Sci. USA 99, 5515-5520, all of which are incorporated by reference herein in their entirety). In this method, a siRNA targeting a CR gene is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid- or virus-based shRNAs can be expressed stably in cells, allowing long-term gene silencing in cells both in vitro and in vivo (see, McCaffrey et al. 2002, Nature 418, 38-39; Xia et al., 2002, Nat. Biotech. 20, 1006-1010; Lewis et al., 2002, Nat. Genetics 32, 107-108; Rubinson et al.; 2003, Nat. Genetics 33, 401-406; Tiscornia et al., 2003, Proc. Natl. Acad. Sci. USA 100, 1844-1848, all of which are incorporated by reference herein in their entirety). Such plasmid- or virus-based shRNAs can be delivered using a gene therapy approach. SiRNAs targeting the CR gene can also be delivered to an organ or tissue in a mammal, such a human, in vivo (see, e.g., Song et al. 2003, Nat. Medicine 9, 347-351; Sorensen et al., 2003, J. Mol. Biol. 327, 761-766; Lewis et al., 2002, Nat. Genetics 32, 107-108, all of which are incorporated by reference herein in their entirety). In this method, a solution of siRNA is injected intravenously into the mammal. The siRNA can then reach an organ or tissue of interest and effectively reduce the expression of the target gene in the organ or tissue of the mammal.

In preferred embodiments, an siRNA pool (mixture) containing at least k (k=2, 3, 4, 5, 6 or 10) different siRNAs targeting a CR gene at different sequence regions is used to silence the gene. In a preferred embodiment, the total siRNA concentration of the pool is about the same as the concentration of a single siRNA when used individually. As used herein, the word “about” with reference to concentration means within 20%. Preferably, the total concentration of the pool of siRNAs is an optimal concentration for silencing the intended target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the composition of the pool, including the number of different siRNAs in the pool and the concentration of each different siRNA, is chosen such that the pool of siRNAs causes less than 30%, 20%, 10% or 5%, 1%, 0.1% or 0.01% of silencing of any off-target genes (e.g., as determined by standard nucleic acid assay, e.g., PCR). In another preferred embodiment, the concentration of each different siRNA in the pool of different siRNAs is about the same. In still another preferred embodiment, the respective concentrations of different siRNAs in the pool are different from each other by less than 5%, 10%, 20% or 50% of the concentration of any one siRNA or said total siRNA concentration of said different siRNAs. In still another preferred embodiment, at least one siRNA in the pool of different siRNAs constitutes more than 90%, 80%, 70%, 50%, or 20% of the total siRNA concentration in the pool. In still another preferred embodiment, none of the siRNAs in the pool of different siRNAs constitutes more than 90%, 80%, 70%, 50%, or 20% of the total siRNA concentration in the pool. In other embodiments, each siRNA in the pool has a concentration that is lower than the optimal concentration when used individually. In a preferred embodiment, each different siRNA in the pool has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the pool has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene. In specific embodiments, a pool containing the 3 different is used for targeting a CR gene. More detailed descriptions of techniques for carrying out RNAi are also presented in Section 5.6.

In other embodiments, antisense, ribozyme, and triple helix forming nucleic acid are designed to inhibit the translation or transcription of a CR protein or gene with minimal effects on the expression of other genes that may share one or more sequence motif with the CR gene. To accomplish this, the oligonucleotides used should be designed on the basis of relevant sequences unique to a CR gene. In one embodiment, the oligonucleotide used specifically inhibits the translation or transcription of a CR protein or gene without substantially affecting the translation or transcription of other proteins in the same protein family.

For example, and not by way of limitation, the oligonucleotides should not fall within those regions where the nucleotide sequence of a CR gene is most homologous to that of other genes. In the case of antisense molecules, it is preferred that the sequence be at least 18 nucleotides in length in order to achieve sufficiently strong annealing to the target mRNA sequence to prevent translation of the sequence. Izant et al., 1984, Cell, 36:1007-1015; Rosenberg et al., 1985, Nature, 313:703-706.

Ribozymes are RNA molecules which possess highly specific endoribonuclease activity. Hammerhead ribozymes comprise a hybridizing region which is complementary in . nucleotide sequence to at least part of the target RNA, and a catalytic region which is adapted to cleave the target RNA. The hybridizing region contains nine (9) or more nucleotides. Therefore, the hammerhead ribozymes are useful for targeting a CR gene having a hybridizing region which is complementary to the sequences of the target gene and are at least nine nucleotides in length. The construction and production of such ribozymes is well known in the art and is described more fully in Haseloff et al., 1988, Nature, 334:585-591.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been et al., 1986, Cell, 47:207-216). The Cech endoribonucleases have an eight base pair active site which hybridizes to a target RNA sequence whereafter cleavage of the target RNA takes place.

In the case of oligonucleotides that hybridize to and form triple helix structures at the 5′ terminus of a CR gene and can be used to block transcription, it is preferred that they be complementary to those sequences in the 5′ terminus of a CR gene which are not present in other related genes. It is also preferred that the sequences not include those regions of the promoter of a CR gene which are even slightly homologous to that of other related genes.

The foregoing compounds can be administered by a variety of methods which are known in the art including, but not limited to the use of liposomes as a delivery vehicle. Naked DNA or RNA molecules may also be used where they are in a form which is resistant to degradation such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to poly-lysine or transferrin. Nucleic acid may also be transported into cells by any of the various viral carriers, including but not limited to, retrovirus, vaccinia, AAV, and adenovirus.

Alternatively, a recombinant nucleic acid molecule which encodes, or is, such antisense nucleic acid, ribozyme, triple helix forming nucleic acid, or nucleic acid molecule of a CR gene can be constructed. This nucleic acid molecule may be either RNA or DNA. If the nucleic acid encodes an RNA, it is preferred that the sequence be operatively attached to a regulatory element so that sufficient copies of the desired RNA product are produced. The regulatory element may permit either constitutive or regulated transcription of the sequence. In vivo, that is, within the cells or cells of an organism, a transfer vector such as a bacterial plasmid or viral RNA or DNA, encoding one or more of the RNAs, may be transfected into cells e.g. (Llewellyn et al., 1987, J. Mol. Biol., 195:115-123; Hanahan et al. 1983, J. Mol. Biol., 166:557-580). Once inside the cell, the transfer vector may replicate, and be transcribed by cellular polymerases to produce the RNA or it may be integrated into the genome of the host cell. Alternatively, a transfer vector containing sequences encoding one or more of the RNAs may be transfected into cells or introduced into cells by way of micromanipulation techniques such as microinjection, such that the transfer vector or a part thereof becomes integrated into the genome of the host cell.

The activity of a CR protein can be modulated by modulating the interaction of a CR protein with its binding partners. In one embodiment, agents, e.g., antibodies, peptides, aptamers, small organic or inorganic molecules, can be used to inhibit binding of a CR protein binding partner to treat cancer. In another embodiment, agents, e.g., antibodies, aptamers, small organic or inorganic molecules, can be used to inhibit the activity of a CR protein to treat cancer. In other embodiments, when the CR protein is a kinase, the invention provides small molecule inhibitors of the CR protein. A small molecule inhibitor is a low molecular weight phosphorylation inhibitor. As used herein, a small molecule refers to an organic or inorganic molecule having a molecular weight is under 1000 Daltons, preferably in the range between 300 to 700 Daltons, which is not a nucleic acid molecule or a peptide molecule. The small molecule can be naturally occurring, e.g., extracted from plant or microorganisms, or non-naturally occurring, e.g., generated de novo by synthesis. A small molecule that is an inhibitor can be used to block a cellular process that dependent on a CR protein. In one embodiment, the inhibitors are substrate mimics. In a preferred embodiment, the inhibitor of the CR proteins is an ATP mimic. In one embodiment, such ATP mimics possess at least two aromatic rings. In a preferred embodiment, the ATP mimic comprises a moiety that forms extensive contacts with residues lining the ATP binding cleft of the CR protein and/or peptide segments just outside the cleft, thereby selectively blocking the ATP binding site of the CR protein. Minor structural differences from ATP can be introduced into the ATP mimic based on the peptide segments just outside the cleft. Such differences can lead to specific hydrogen bonding and hydrophobic interactions with the peptide segments just outside the cleft.

In still other embodiments, antibodies that specifically bind the CR protein are used. In a preferred embodiment, the invention provides antibodies that specifically bind the extracellular domain of a CR protein that is a receptor. Antibodies that specifically bind a target can be obtained using standard method known in the art, e.g., a method described in Section 5.8.

In one embodiment, an antibody-drug conjugate comprising an antibody that specifically binds a cell surface expressed CR protein is used. The efficacy of the antibodies that targets CR protein can be increased by attaching toxins to them. Existing immunotoxins are based on bacterial toxins like pseudomonas exotoxin, plant exotoxin like ricin or radio-nucleotides. The toxins are chemically conjugated to a specific ligand such as the variable domain of the heavy or light chain of the monoclonal antibody. Normal cells lacking the cancer specific antigens are not targeted by the targeted antibody.

In other embodiments, a peptide and peptidomimetic that interferes with the interaction of a CR protein with its interaction partner is used. A peptide preferably has a size of at least 5, 10, 15, 20 or 30 amino acids. Such a peptide or peptidomimetic can be designed by a person skilled in the art based on the sequence and structure of a CR protein. In one embodiment, a peptide or peptidomimetic that interferes with substrate binding of a CR protein is used. In another embodiment, peptide or peptidomimetic that interferes with the binding of a signal molecule to a CR protein is used. In some embodiments of the invention, a fragment or polypeptide of at least 5, 10, 20, 50, 100 amino acids in length of a CR protein are used.

In another embodiment, a dominant negative mutant of a CR protein is used to reduce activity of a CR protein. Such a dominant negative mutant can be designed by a person skilled in the art based on the sequence and structure of a CR protein. In one embodiment, a dominant negative mutant that interferes with substrate binding of a CR protein is used. In another embodiment, a dominant negative mutant that interferes with the binding of a signal molecule to a CR protein is used. In a preferred embodiment, the invention provides a dominant negative mutant that comprises the C-terminal region of a CR protein. In another embodiment, the invention provides a dominant negative mutant that comprises the N-terminal region of the CR protein.

Gene therapy can be used for delivering any of the above described nucleic acid and protein/peptide therapeutics into target cells. Gene therapy is particularly useful for enhancing aberrantly down-regulated genes. Exemplary methods for carrying out gene therapy are described below. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, New York; and Kriegler, 1990, Gene Transfer and Expression, A Laboratory Manual, Stockton Press, New York.

In a preferred embodiment, the therapeutic comprises a nucleic acid that is part of an expression vector that expresses the therapeutic nucleic acid or peptide/polypeptide in a suitable host. In particular, such a nucleic acid has a promoter operably linked to the coding region, said promoter being inducible or constitutive, and, optionally, tissue-specific. In another particular embodiment, a nucleic acid molecule is used in which the coding sequences and any other desired sequences are flanked by regions that promote homologous recombination at a desired site in the genome, thus providing for intrachromosomal expression of the CR nucleic acid (see e.g., Koller and Smithies, 1989, Proc. Natl. Acad. Sci. U.S.A. 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. U.S.A. 86:8932-8935; Zijlstra et al., 1989, Nature 342:435-438).

In a specific embodiment, a viral vector that contains the nucleic acid of a CR gene is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The CR nucleic acid to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. Genet. and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993, Current Opinion in Genetics and Development 3:499-503) present a review of adenovirus-based gene therapy. Bout et al. (1994, Human Gene Therapy 5:3-10) demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234.

Adeno-associated virus (AAV) has also been proposed for use in gene therapy (Walsh et al., 1993, Proc. Soc. Exp. Biol. Med. 204:289-300).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled person in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a nucleic acid is introduced into the cells such that it is expressible by the cells or their progeny, and the recombinant cells are then administered in vivo for therapeutic effect. In a specific embodiment, stem or progenitor cells are used. Such stem cells can be hematopoietic stem cells (HSC).

Any technique which provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see e.g., Kodo et al., 1984, J. Clin. Invest. 73:1377-1384). The HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. U.S.A. 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The methods and/or compositions described above for modulating the expression and/or activity of a CR gene or CR protein may be used to treat patients in conjunction with a chemotherapeutic agent, e.g., GleevecTM.

The effects or benefits of administration of the compositions of the invention alone or in conjunction with a chemotherapeutic agent can be evaluated by any methods known in the art, e.g., by methods that are based on measuring the survival rate, side effects, dosage requirement of the chemotherapeutic agent, or any combinations thereof. If the administration of the compositions of the invention achieves any one or more benefits in. a patient, such as increasing the survival rate, decreasing side effects, lowering the dosage requirement for the chemotherapeutic agent, the compositions of the invention are said to have augmented a chemotherapy treatment, and the method is said to have efficacy.

### 5.5. Methods for Screening Agents that Modulate CR Proteins

Agents that modulate the expression or activity of a chemotherapy response gene or encoded protein, or modulate interaction of a chemotherapy response protein with other proteins or molecules can be identified using a method described in this section. Such agents are useful in treating cancer patients who exhibit non-responsiveness to chemotherapy. The methods described in this section can be performed in vivo, e.g., using cells that are in vivo. The methods described in this section can also be performed in vitro, e.g., using cells in a cell culture.

### 5.5.1. Screening Assays

The following assays are designed to identify compounds that bind to a chemotherapy response gene or its products, bind to other cellular proteins that interact with a chemotherapy response protein, bind to cellular constituents, e.g., proteins, that are affected by a chemotherapy response protein, or bind to compounds that interfere with the interaction of the chemotherapy response gene or its product with other cellular proteins and to compounds which modulate the expression or activity of a chemotherapy response gene (i.e., modulate the expression level of the chemotherapy response gene and/or modulate the activity level of the chemotherapy response protein). Assays may additionally be utilized which identify compounds which bind to chemotherapy response protein regulatory sequences (e.g., promoter sequences), see e.g., Platt, K.A., 1994, J. Biol. Chem. 269:28558-28562, which is incorporated herein by reference in its entirety, which may modulate the level of chemotherapy response gene expression. Compounds may include, but are not limited to, small organic molecules which are able to affect expression of the chemotherapy response gene or some other gene involved in the chemotherapy response protein pathways, or other cellular proteins. Further, among these compounds are compounds which affect the level of chemotherapy response gene expression and/or chemotherapy response protein activity and which can be used in the regulation of sensitivity to the effect of a chemotherapy agent.

Compounds may include, but are not limited to, peptides such as, for example, soluble peptides, including but not limited to, Ig-tailed fusion peptides, and members of random peptide libraries (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, Z. et al., 1993, Cell 72:767-778), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules.

Compounds identified via assays such as those described herein may be useful, for example, in modulating the biological function of the chemotherapy response protein.

In vitro systems may be designed to identify compounds capable of binding a chemotherapy response protein. Compounds identified may be useful, for example, in modulating the activity of wild type and/or mutant chemotherapy response protein, may be useful in elaborating the biological function of the chemotherapy response protein, may be utilized in screens for identifying compounds that disrupt normal chemotherapy response protein interactions, or may in themselves disrupt such interactions.

The principle of the assays used to identify compounds that bind to the chemotherapy response protein involves preparing a reaction mixture of the chemotherapy response protein and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring chemotherapy response protein or the test substance onto a solid phase and detecting chemotherapy response protein/test compound complexes anchored on the solid phase at the end of the reaction. In one embodiment of such a method, the chemotherapy response protein may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for a chemotherapy response protein or the test compound to anchor any complexes formed in solution, and a labeled antibody specific for the other component of the possible complex to detect anchored complexes.

The chemotherapy response gene or chemotherapy response protein may interact in vivo with one or more intracellular or extracellular molecules, such as proteins. For purposes of this discussion, such molecules are referred to herein as “binding partners”. Compounds that disrupt chemotherapy response protein binding may be useful in modulating the activity of the chemotherapy response protein. Compounds that disrupt chemotherapy response gene binding may be useful in modulating the expression of the chemotherapy response gene, such as by modulating the binding of a regulator of chemotherapy response gene. Such compounds may include, but are not limited to molecules such as peptides which would be capable of gaining access to the chemotherapy response protein.

The basic principle of the assay systems used to identify compounds that interfere with the interaction between the chemotherapy response protein and its intracellular or extracellular binding partner or partners involves preparing a reaction mixture containing the chemotherapy response protein, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of a chemotherapy response protein and its binding partner. Control reaction mixtures are incubated without the test compound or with a placebo. The formation of any complexes between the chemotherapy response protein and the binding partner is then detected. The formation of a complex in the control reaction, but not in the reaction mixture containing the test compound, indicates that the compound interferes with the interaction of the chemotherapy response protein and the interactive binding partner. Additionally, complex formation within reaction mixtures containing the test compound and a normal chemotherapy response protein may also be compared to complex formation within reaction mixtures containing the test compound and a mutant chemotherapy response protein. This comparison may be important in those cases where it is desirable to identify compounds that disrupt interactions of mutant but hot the normal chemotherapy response protein.

The assay for compounds that interfere with the interaction of the chemotherapy response proteins and binding partners can be conducted in a heterogeneous or homogeneous format. Heterogeneous assays involve anchoring either the chemotherapy response protein or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the chemotherapy response proteins and the binding partners, e.g., by competition, can be identified by conducting the reaction in the presence of the test substance; i.e., by adding the test substance to the reaction mixture prior to or simultaneously with the chemotherapy response protein and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the chemotherapy response protein or its interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the chemotherapy response protein or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternative embodiment, a homogeneous assay can be used. In this approach, a preformed complex of the chemotherapy response protein and the interactive binding partner is prepared in which either the chemotherapy response protein or its binding partners is labeled, but the signal generated by the label is quenched due to complex formation (see, e.g., U.S. Pat. No. 4,109,496 which utilizes this approach for immunoassays). The addition of a test substance that competes with and displaces one of the species from the preformed complex will result in the generation of a signal above background. In this way, test substances which disrupt chemotherapy response protein/binding partner interaction can be identified.

In a particular embodiment, the chemotherapy response protein can be prepared for immobilization using recombinant DNA techniques. For example, the coding region of chemotherapy response gene can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with the radioactive isotope ¹²⁵I, for example, by methods routinely practiced in the art. In a heterogeneous assay, e.g., the GST-chemotherapy response protein fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the chemotherapy response protein and the interactive binding partner can be detected by measuring the amount of radioactivity that remains associated with the glutathione-agarose beads. A successful inhibition of the interaction by the test compound will result in a decrease in measured radioactivity.

Alternatively, the GST-chemotherapy response protein fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the chemotherapy response protein/binding partner interaction can be detected by adding the labeled antibody and measuring the radioactivity associated with the beads.

In another embodiment of the invention, these same techniques can be employed using peptide fragments that correspond to the binding domains of the chemotherapy response protein and/or the interactive binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this section above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the binding partner is obtained, short gene segments can be engineered to express peptide fragments of the protein, which can then be tested for binding activity and purified or synthesized.

For example, and not by way of limitation, a chemotherapy response protein can be anchored to a solid material as described in this section, above, by making a GST-chemotherapy response protein fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-chemotherapy response protein fusion protein and allowed to bind. After washing away unbound peptides, labeled bound material, representing the binding partner binding domain, can be eluted, purified, and analyzed for amino acid sequence by well-known methods. Peptides so identified can be produced synthetically or fused to appropriate facilitative proteins using recombinant DNA technology.

Some chemotherapy response proteins are kinases. Kinase activity of a chemotherapy response protein can be assayed in vitro using a synthetic peptide substrate of a chemotherapy response protein of interest, e.g., a GSK-derived biotinylated peptide substrate. The phosphopeptide product is quantitated using a Homogenous Time-Resolved Fluorescence (HTRF) assay system (Park et al., 1999, Anal. Biochem. 269:94-104). The reaction mixture contains suitable amounts of ATP, peptide substrate, and the chemotherapy response protein. The peptide substrate has a suitable amino acid sequence and is biotinylated at the N-terminus. The kinase reaction is incubated, and then terminated with Stop/Detection Buffer and GSK3α anti-phosphoserine antibody (e.g., Cell Signaling Technologies, Beverly, Mass.; Cat #9338) labeled with europium-chelate (e.g., from Perkin Elmer, Boston, Mass.). The reaction is allowed to equilibrate, and relative fluorescent units are determined. Inhibitor compounds are assayed in the reaction described above, to determine compound IC50s. A particular compound is added to in a half-log dilution series covering a suitable range of concentrations, e.g., from 1 nM to 100 Relative phospho substrate formation, read as HTRF fluorescence units, is measured over the range of compound concentrations and a titration curve generated using a four parameter sigmoidal fit. Specific compounds having IC₅₀ below a predetermined threshold value, e.g., ≦50 μM against a substrate, can be identified.

The extent of peptide phosphorylation can be determined by Homogeneous Time Resolved Fluorescence (HTRF) using a lanthanide chelate (Lance)-coupled monoclonal antibody specific for the phosphopeptide in combination with a streptavidin-linked allophycocyanin (SA-APC) fluorophore which binds to the biotin moiety on the peptide. When the Lance and APC are in proximity (i.e. bound to the same phosphopeptide molecule), a non-radiative energy transfer takes place from the Lance to the APC, followed by emission of light from APC at 665 nm. The assay can be run using various assay format, e.g., streptavidin flash plate assay, streptavidin filter plate assay.

A standard PICA assay can be used to assay the activity of protein kinase A (PICA). A standard PKC assay can be used to assay the activity of protein kinase C (PKC). The most common methods for assaying PKA or PKC activity involves measuring the transfer of ³²P-labeled phosphate to a protein or peptide substrate that can be captured on phosphocellulose filters via weak electrostatic interactions.

Kinase inhibitors can be identified using fluorescence polarization to monitor kinase activity. This assay utilizes GST-chemotherapy response protein, peptide substrate, peptide substrate tracer, an anti-phospho monoclonal IgG, and the inhibitor compound. Reactions are incubated for a period of time and then terminated. Stopped reactions are incubated and fluorescence polarization values determined.

In a specific embodiment, a standard SPA Filtration Assay and FlashPlate® Kinase Assay can be used to measure the activity of a chemotherapy response protein. In these assays, GST-chemotherapy response protein, biotinylated peptide substrate, ATP, and ³³P-γ-ATP are allowed to react. After a suitable period of incubation, the reactions are terminated. In a SPA Filtration Assay, peptide substrate is allowed to bind Scintilation proximity assay (SPA) beads (Amersham Biosciences), followed by filtration on a Packard GF/B Unifilter plate and washed with phosphate buffered saline. pried plates are sealed and the amount of ³³P incorporated into the peptide substrate is determined. In a FlashPlate® Kinase Assay, a suitable amount of the reaction is transferred to streptavidin-coated FlashPlates® (NEN) and incubated. Plates are washed, dried, sealed and the amount of ³³P incorporated into the peptide substrate is determined.

A standard DELFIA® Kinase Assay can also be used. In a DELFIA® Kinase Assay, GST-chemotherapy response protein, peptide substrate, and ATP are allowed to react. After the reactions are terminated, the biotin-peptide substrates are captured in the stopped reactions. Wells are washed and reacted with anti-phospho polyclonal antibody and europium labeled anti-rabbit-IgG. Wells are washed and europium released from the bound antibody is detected.

Other assays, such as those described in WO 04/080973, WO 02/070494, and WO 03/101444, may also be utilized to determine biological activity of the instant compounds.

### 5.5.2. Screening Compounds that Modulate Expression or Activity of a Gene and/or its Products

For chemotherapy response genes that are kinases, inhibitor compounds can be assayed for their ability to inhibit a chemotherapy response protein by monitoring the phosphorylation or autophosphorylation in response to the compound. Cells are grown in culture medium. Cells are pooled, counted, seeded into 6 well dishes at 200,000 cells per well in 2 ml media, and incubated. Serial dilution series of compounds or control are added to each well and incubated. Following the incubation period, each well is washed and Protease Inhibitor Cocktail Complete is added to each well. Lysates are then transferred to microcentrifuge tubes and frozen at −80° C. Lysates are thawed on ice and cleared by centrifugation and the supernatants are transferred to clean tubes. Samples are electorphoresed and proteins are transferred onto PVDF. Blots are then blocked and probed using an antibody against phospho-serine or phospho threonine. Bound antibody is visualized using a horseradish peroxidase conjugated secondary antibody and enhanced chemiluminescence. After stripping of the first antibody set, blots are re-probed for total chemotherapy response protein, using a monoclonal antibody specific for the chemotherapy response protein. The chemotherapy response protein monoclonal is detected using a sheep anti-mouse IgG coupled to horseradish peroxidase and enhanced chemiluminescence. ECL exposed films are scanned and the intensity of specific bands is quantitated. Titrations are evaluated for level of phosphor-Ser signal normalized to total chemotherapy response protein and IC50 values are calculated.

Detection of phosphonucleolin in cell lysates can be carried out using biotinylated anti-nucleolin antibody and ruthenylated goat anti-mouse antibody. To each well of a 96-well plate is added biotynylated anti-nucleolin antibody and streptavidin coated paramagnetic beads, along with a suitable cell lysate. The antibodies and lysate are incubated. Next, another anti-phosphonucleolin antibody are added to each well of the lysate mix and incubated. Lastly, the ruthenylated goat anti-mouse antibody in antibody buffer is added to each well and incubated. The lysate antibody mixtures are read and EC50s for compound dependent increases in phosphor-nucleolin are determined.

The compounds identified in the screen include compounds that demonstrate the ability to selectively modulate the expression or activity of a chemotherapy response gene or its encoded protein. These compounds include but are not limited to siRNA, antisense nucleic acid, ribozyme, triple helix forming nucleic acid, antibody, and polypeptide molecules, aptamrs, and small organic or inorganic molecules.

### 5.6. Methods of Performing RNA Interference

Any method known in the art for gene silencing can be used in the present invention (see, e.g., Guo et al., 1995, Cell 81:611-620; Fire et al., 1998, Nature 391:806-811; Grant, 1999, Cell 96:303-306; Tabara et al., 1999, Cell 99:123-132; Zamore et al., 2000, Cell 101:25-33; Bass, 2000, Cell 101:235-238; Petcherski et al., 2000, Nature 405:364-368; Elbashir et al., Nature 411:494-498; Paddison et al., Proc. Natl. Acad. Sci. USA 99:1443-1448). The siRNAs targeting a gene can be designed according to methods known in the art (see, e.g., International Application Publication No. WO 2005/018534, published on Mar. 3, 2005, and Elbashir et al., 2002, Methods 26:199-213, each of which is incorporated herein by reference in its entirety).

An siRNA having only partial sequence homology to a target gene can also be used (see, e.g., International Application Publication No. WO 2005/018534, published on Mar. 3, 2005, which is incorporated herein by reference in its entirety). In one embodiment, an siRNA that comprises a sense strand contiguous nucleotide sequence of 11-18 nucleotides that is identical to a sequence of a transcript of a gene but the siRNA does not have full length homology to any sequences in the transcript is used to silence the gene. Preferably, the contiguous nucleotide sequence is in the central region of the siRNA molecules. A contiguous nucleotide sequence in the central region of an siRNA can be any continuous stretch of nucleotide sequence in the siRNA which does not begin at the 3′ end. For example, a contiguous nucleotide sequence of 11 nucleotides can be the nucleotide sequence 2-12, 3-13, 4-14, 5-15, 6-16, 7-17, 8-18, or 9-19. In preferred embodiments, the contiguous nucleotide sequence is 11-16, 11-15, 14-15, 11, 12, or 13 nucleotides in length.

In another embodiment, an siRNA that comprises a 3′ sense strand contiguous nucleotide sequence of 8-18 nucleotides which is identical to a sequence of a transcript of a gene but which siRNA does not have full length sequence identity to any contiguous sequences in the transcript is used to silence the gene. In this application, a 3′ 8-18 nucleotide sequence is a continuous stretch of nucleotides that begins at the first paired base, i.e., it does not comprise the two base 3′ overhang. Thus, when it is stated that a particular nucleotide sequence is at the 3′ end of the siRNA, the 2 base overhang is not considered. In preferred embodiments, the contiguous nucleotide sequence is 8-16, 8-15, 8-12, 11, 10, 9, or 8 nucleotides in length.

An siRNA having only partial sequence homology to its target genes is especially useful for silencing a plurality of different genes in a cell. In one embodiment, an siRNA is used to silence a plurality of different genes, the transcript of each of the genes comprises a nucleotide sequence that is identical to a central contiguous nucleotide sequence of at least 11 nucleotides of the sense strand or the antisense strand of the siRNA, and/or comprises a nucleotide sequence that is identical to a contiguous nucleotide sequence of at least 9 nucleotides at the 3′ end of the sense strand or the antisense strand of the siRNA. In preferred embodiments, the central contiguous nucleotide sequence is 11-15, 14-15, 11, 12, or 13 nucleotides in length. In other preferred embodiments, the 3′ contiguous nucleotide sequence is 8-15, 8-12, 11, 10, 9, or 8 nucleotides in length.

In one embodiment, in vitro siRNA transfection is carried out as follows: one day prior to transfection, 100 microliters of chosen cells, e.g., cervical cancer HeLa cells (ATCC, Cat. No. CCL-2), grown in DMEM/10% fetal bovine serum (Invitrogen, Carlsbad, Calif.) to approximately 90% confluency are seeded in a 96-well tissue culture plate (Corning, Corning, N.Y.) at 1500 cells/well. For each transfection 85 microliters of OptiMEM (Invitrogen) is mixed with 5 microliter of serially diluted siRNA (Dharma on, Denver) from a 20 micro molar stock. For each transfection 5 microliter OptiMEM is mixed with 5 microliter Oligofectamine reagent (Invitrogen) and incubated 5 minutes at room temperature. The 10 microliter OptiMEM/Oligofectamine mixture is dispensed into each tube with the OptiMEM/siRNA mixture, mixed and incubated 15-20 minutes at room temperature. 10 microliter of the transfection mixture is aliquoted into each well of the 96-well plate and incubated for 4 hours at 37° C. and 5% CO₂.

In preferred embodiments, an siRNA pool containing at least k (k=2, 3, 4, 5, 6 or 10) different siRNAs targeting the secondary target gene at different sequence regions is used to transfect the cells. In another preferred embodiment, an siRNA pool containing at least k (k=2, 3, 4, 5, 6 or 10) different siRNAs targeting two or more different target genes is used to transfect the cells.

In a preferred embodiment, the total siRNA concentration of the pool is about the same as the concentration of a single siRNA when used individually, e.g., 100 nM. Preferably, the total concentration of the pool of siRNAs is an optimal concentration for silencing the intended target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the composition of the pool, including the number of different siRNAs in the pool and the concentration of each different siRNA, is chosen such that the pool of siRNAs causes less than 30%, 20%, 10% or 5%, 1%, 0.1% or 0.01% of silencing of any off-target genes (e.g., as determined by standard nucleic acid assay, e.g., PCR). In another preferred embodiment, the concentration of each different siRNA in the pool of different siRNAs is about the same. In still another preferred embodiment, the respective concentrations of different siRNAs in the pool are different from each other by less than 5%, 10%, 20% or 50% of the concentration of any one siRNA or said total siRNA concentration of said different siRNAs. In still another preferred embodiment, at least one siRNA in the pool of different siRNAs constitutes more than 90%, 80%, 70%, 50%, or 20% of the total siRNA concentration in the pool. In still another preferred embodiment, none of the siRNAs in the pool of different siRNAs constitutes more than 90%, 80%, 70%, 50%, or 20% of the total siRNA concentration in the pool. In other embodiments, each siRNA in the pool has an concentration that is lower than the optimal concentration when used individually. In a preferred embodiment, each different siRNA in the pool has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the pool has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene.

Another method for gene silencing is to introduce an shRNA, for short hairpin RNA (see, e.g., Paddison et al., 2002, Genes Dev. 16, 948-958; Brummelkamp et al., 2002, Science 296, 550-553; Sui, G. et al. 2002, Proc. Natl. Acad. Sci. USA 99, 5515-5520, all of which are incorporated by reference herein in their entirety), which can be processed in the cells into siRNA. In this method, a desired siRNA sequence is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid-based shRNAs can be expressed stably in cells, allowing long-term gene silencing in cells both in vitro and in vivo, e.g., in animals (see, McCaffrey et al. 2002, Nature 418, 38-39; Xia et al., 2002, Nat. Biotech. 20, 1006-1010; Lewis et al., 2002, Nat. Genetics 32, 107-108; Rubinson et al., 2003, Nat. Genetics 33, 401-406; Tiscornia et al., 2003, Proc. Natl. Acad. Sci. USA 100, 1844-1848, all of which are incorporated by reference herein in their entirety). Thus, in one embodiment, a plasmid-based shRNA is used.

In a preferred embodiment, shRNAs are expressed from recombinant vectors introduced either transiently or stably integrated into the genome (see, e.g., Paddison et al., 2002, Genes Dev 16:948-958; Sui et al., 2002, Proc Natl Acad Sci USA 99:5515-5520; Yu et al., 2002, Proc Natl Acad Sci USA 99:6047-6052; Miyagishi et al., 2002, Nat Biotechnol 20:497-500; Paul et al., 2002, Nat Biotechnol 20:505-508; Kwak et al., 2003, J Pharmacol Sci 93:214-217; Brummelkamp et al., 2002, Science 296:550-553; Boden et al., 2003, Nucleic Acids Res 31:5033-5038; Kawasaki et al., 2003, Nucleic Acids Res 31:700-707). The siRNA that disrupts the target gene can be expressed (via an shRNA) by any suitable vector which encodes the shRNA. The vector can also encode a marker which can be used for selecting clones in which the vector or a sufficient portion thereof is integrated in the host genome such that the shRNA is expressed. Any standard method known in the art can be used to deliver the vector into the cells. In one embodiment, cells expressing the shRNA are generated by transfecting suitable cells with a plasmid containing the vector. Cells can then be selected by the appropriate marker. Clones are then picked, and tested for knockdown. In a preferred embodiment, the expression of the shRNA is under the control of an inducible promoter such that the silencing of its target gene can be turned on when desired. Inducible expression of an siRNA is particularly useful for targeting essential genes.

In one embodiment, the expression of the shRNA is under the control of a regulated promoter that allows tuning of the silencing level of the target gene. This allows screening against cells in which the target gene is partially knocked out. As used herein, a “regulated promoter” refers to a promoter that can be activated when an appropriate inducing agent is present. An “inducing agent” can be any molecule that can be used to activate transcription by activating the regulated promoter. An inducing agent can be, but is not limited to, a peptide or polypeptide, a hormone, or an organic small molecule. An analogue of an inducing agent, i.e., a molecule that activates the regulated promoter as the inducing agent does, can also be used. The level of activity of the regulated promoter induced by different analogues may be different, thus allowing more flexibility in tuning the activity level of the regulated promoter. The regulated promoter in the vector can be any mammalian transcription regulation system known in the art (see, e.g., Gossen et al, 1995, Science 268:1766-1769; Lucas et al, 1992, Annu. Rev. Biochem. 61:1131; Li et al., 1996, Cell 85:319-329; Saez et al., 2000, Proc. Natl. Acad. Sci. USA 97:14512-14517; and Pollock et al., 2000, Proc. Natl. Acad. Sci. USA 97:13221-13226). In preferred embodiments, the regulated promoter is regulated in a dosage and/or analogue dependent manner. In one embodiment, the level of activity of the regulated promoter is tuned to a desired level by a method comprising adjusting the concentration of the inducing agent to which the regulated promoter is responsive. The desired level of activity of the regulated promoter, as obtained by applying a particular concentration of the inducing agent, can be determined based on the desired silencing level of the target gene.

In one embodiment, a tetracycline regulated gene expression system is used (see, e.g., Gossen et al, 1995, Science 268:1766-1769; U.S. Pat. No. 6,004,941). A tet regulated system utilizes components of the tet repressor/operator/inducer system of prokaryotes to regulate gene expression in eukaryotic cells. Thus, the invention provides methods for using the tet regulatory system for regulating the expression of an shRNA linked to one or more tet operator sequences. The methods involve introducing into a cell a vector encoding a fusion protein that activates transcription. The fusion protein comprises a first polypeptide that binds to a tet operator sequence in the presence of tetracycline or a tetracycline analogue operatively linked to a second polypeptide that activates transcription in cells. By modulating the concentration of a tetracycline, or a tetracycline analogue, expression of the tet operator-linked shRNA is regulated.

In other embodiments, an ecdyson regulated gene expression system (see, e.g., Saez et al., 2000, Proc. Natl. Acad. Sci. USA 97:14512-14517), or an MMTV glucocorticoid response element regulated gene expression system (see, e.g., Lucas et al, 1992, Annu. Rev. Biochem. 61:1131) may be used to regulate the expression of the shRNA.

In one embodiment, the pRETRO-SUPER (pRS) vector which encodes a puromycin-resistance marker and drives shRNA expression from an H1 (RNA Pol III) promoter is used. The pRS-shRNA plasmid can be generated by any standard method known in the art. In one embodiment, the pRS-shRNA is deconvoluted from the library plasmid pool for a chosen gene by transforming bacteria with the pool and looking for clones containing only the plasmid of interest. Preferably, a 19 mer siRNA sequence is used along with suitable forward and reverse primers for sequence specific PCR. Plasmids are identified by sequence specific PCR, and confirmed by sequencing. Cells expressing the shRNA are generated by transfecting suitable cells with the pRS-shRNA plasmid. Cells are selected by the appropriate marker, e.g., puromycin, and maintained until colonies are evident. Clones are then picked, and tested for knockdown. In another embodiment, an shRNA is expressed by a plasmid, e.g., a pRS-shRNA. The knockdown by the pRS-shRNA plasmid, can be achieved by transfecting cells using Lipofectamine 2000 (Invitrogen).

In yet another method, siRNAs can be delivered to an organ or tissue in an animal, such a human, in vivo (see, e.g., Song et al. 2003, Nat. Medicine 9, 347-351; Sorensen et al., 2003, J. Mol. Biol. 327, 761-766; Lewis et al., 2002, Nat. Genetics 32, 107-108, all of which are incorporated by reference herein in their entirety). In this method, a solution of siRNA is injected intravenously into the animal. The siRNA can then reach an organ or tissue of interest and effectively reduce the expression of the target gene in the organ or tissue of the animal.

### 5.7. Production of CR Proteins and Peptides

Chemotherapy response proteins, or peptide fragments thereof, can be prepared for uses according to the present invention. For example, chemotherapy response proteins, or peptide fragments thereof, can be used for the generation of antibodies, in diagnostic assays, for screening of inhibitors, or for the identification of other cellular gene products involved in the regulation of expression and/or activity of a chemotherapy response gene.

The chemotherapy response proteins or peptide fragments thereof, may be produced by recombinant DNA technology using techniques well known in the art. The amino acid sequences of the chemotherapy response proteins are well-known and can be obtained from, e.g., GenBank®. Methods which are well known to those skilled in the art can be used to construct expression vectors containing chemotherapy response protein coding sequences and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding chemotherapy response protein sequences may be chemically synthesized using, for example, synthesizers. See, for example, the techniques described in “Oligonucleotide Synthesis”, 1984, Gait, M. J. ed., IRL Press, Oxford, which is incorporated herein by reference in its entirety.

A variety of host-expression vector systems may be utilized to express the chemotherapy response gene coding sequences. Such host-expression systems represent vehicles by which the coding sequences of interest may be produced and subsequently purified, but also represent cells which may, when transformed or transfected with the appropriate nucleotide coding sequences, exhibit the chemotherapy response protein in situ. These include but are not limited to microorganisms such as bacteria (e.g., E. coli, B. subtilis) transformed with recombinant bacteriophage DNA, plasmid DNA or cosmid DNA expression vectors containing chemotherapy response protein coding sequences; yeast (e.g., Saccharomyces, Pichia) transformed with recombinant yeast expression vectors containing the chemotherapy response protein coding sequences; insect cell systems infected with recombinant virus expression vectors (e.g., baculovirus) containing the chemotherapy response protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing chemotherapy response protein coding sequences; or mammalian cell systems (e.g., COS, CHO, BHK, 293, 3T3, N2a) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the chemotherapy response protein being expressed. For example, when a large quantity of such a protein is to be produced, for the generation of pharmaceutical compositions of chemotherapy response protein or for raising antibodies to chemotherapy response protein, for example, vectors which direct the expression of high levels of fusion protein products that are readily purified may be desirable. Such vectors include, but are not limited, to the E. coli expression vector pUR278 (Ruther et al., 1983, EMBO J. 2:1791), in which the chemotherapy response protein coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The chemotherapy response gene coding sequence may be cloned individually into non-essential regions (for example the polyhedrin gene) of the virus and placed under control of an AcNPV promoter (for example the polyhedrin promoter). Successful insertion of chemotherapy response gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the chemotherapy response gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing chemotherapy response protein in infected hosts. (E.g., See Logan & Shenk, 1984, Proc. Natl. Acad. Sci. USA 81:3655-3659). Specific initiation signals may also be required for efficient translation of inserted chemotherapy response protein coding sequences. These signals include the ATG initiation codon and adjacent sequences. In cases where an entire chemotherapy response gene, including its own initiation codon and adjacent sequences, is inserted into the appropriate expression vector, no additional translational control signals may be needed. However, in cases where only a portion of the chemotherapy response gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38.

For long-term, high-yield production of recombinant proteins, stable expression is preferred. For example, cell lines which stably express the chemotherapy response protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the chemotherapy response protein. Such engineered cell lines may be particularly useful in screening and evaluation of compounds that affect the endogenous activity of the chemotherapy response protein.

In another embodiment, the expression characteristics of an endogenous gene (e.g., a chemotherapy response gene) within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene (e.g., a chemotherapy response gene) and controls, modulates, activates, or inhibits the endogenous gene. For example, endogenous chemotherapy response genes which are normally “transcriptionally silent”, i.e., a chemotherapy response gene which is normally not expressed, or is expressed only at very low levels in a cell line or microorganism, may be activated by inserting a regulatory element which is capable of promoting the expression of the gene product in that cell line or microorganism. Alternatively, transcriptionally silent, endogenous chemotherapy response genes may be activated by insertion of a promiscuous regulatory element that works across cell types.

A heterologous regulatory element may be inserted into a stable cell line or cloned microorganism, such that it is operatively linked with and activates or inhibits expression of endogenous chemotherapy response genes, using techniques, such as targeted homologous recombination, which are well known to those of skill in the art, and described e.g., in Chappel, U.S. Pat. No. 5,272,071; PCT Publication No. WO 91/06667 published May 16, 1991; Skoultchi, U.S. Pat. No. 5,981,214; and Treco et al U.S. Pat. No. 5,968,502 and PCT Publication No. WO 94/12650 published Jun. 9, 1994. Alternatively, non-targeted, e.g. non-homologous recombination techniques may be used which are well-known to those of skill in the art and described, e.g., in PCT Publication No. WO 99/15650 published Apr. 1, 1999.

Chemotherapy response gene activation (or inactivation) may also be accomplished using designer transcription factors using techniques well known in the art. Briefly, a designer zinc finger protein transcription factor (ZFP-TF) is made which is specific for a regulatory region of the chemotherapy response gene to be activated or inactivated. A construct encoding this designer ZFP-TF is then provided to a host cell in which the chemotherapy response gene is to be controlled. The construct directs the expression of the designer ZFP-TF protein, which in turn specifically modulates the expression of the endogenous chemotherapy response gene. The following references relate to various aspects of this approach in further detail: Wang & Pabo, 1999, Proc. Natl. Acad. Sci. USA 96, 9568; Berg, 1997, Nature Biotechnol. 15, 323; Greisman & Pabo, 1997, Science 275, 657; Berg & Shi, 1996, Science 271, 1081; Rebar & Pabo, 1994, Science 263, 671; Rhodes & Klug, 1993, Scientific American 269, 56; Pavletich & Pabo, 1991, Science 252, 809; Liu et al., 2001, J. Biol. Chem. 276, 11323; Zhang et al., 2000, J. Biol. Chem. 275, 33850; Beerli et al., 2000, Proc. Natl. Acad. Sci. USA 97, 1495; Kang et al., 2000, J. Biol. Chem. 275, 8742; Beerli et al., 1998, Proc. Natl. Acad. Sci. USA 95, 14628; Kim & Pabo, 1998, Proc. Natl. Acad. Sci. USA 95, 2812; Choo et al., 1997, J. Mol. Biol. 273, 525; Kim & Pabo, 1997, J. Biol. Chem. 272, 29795; Liu et al, 1997, Proc. Natl. Acad. Sci. USA 94, 5525; Kim et al, 1997, Proc. Natl. Acad. Sci. USA 94, 3616; Kikyo et al., 2000, Science 289, 2360; Robertson & Wolffe, 2000, Nature Reviews 1, 11; and Gregory, 2001, Curr. Opin. Genet. Devt.11:142.

A number of selection systems may be used, including but not limited to the herpes simplex virus thymidine kinase (Wigler, et al., 1977, Cell 11:223), hypoxanthine-guanine phosphoribosyl transferase (Szybalska & Szybalski, 1962, Proc. Natl. Acad. Sci. USA 48:2026), and adenine phosphoribosyl transferase (Lowy, et al., 1980, Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺•nitriloacetic acid-agarose columns and histidine-tagged proteins are selectively eluted with imidazole-containing buffers.

In a specific embodiment, recombinant human chemotherapy response proteins can be expressed as a fusion protein with glutathione S-transferase at the amino-terminus (GST-chemotherapy response protein) using standard baculovirus vectors and a (Bac-to-Bac®) insect cell expression system purchased from GIBCO™ Invitrogen. Recombinant protein expressed in insect cells can be purified using glutathione sepharose (Amersham Biotech) using standard procedures described by the manufacturer.

### 5.8. Production of Antibodies that Bind a CR Protein

Chemotherapy response protein or a fragment thereof can be used to raise antibodies which bind chemotherapy response protein. Such antibodies include but are not limited to polyclonal, monoclonal, chimeric, single chain, Fab fragments, and an Fab expression library. In a preferred embodiment, anti chemotherapy response protein C-terminal antibodies are raised using an appropriate C-terminal fragment of a chemotherapy response protein, e.g., the kinase domain. Such antibodies bind the kinase domain of the chemotherapy response protein. In another preferred embodiment, anti chemotherapy response protein N-terminal antibodies are raised using an appropriate N-terminal fragment of a chemotherapy response protein. The N-terminal domain of a chemotherapy response protein is less homologous to other kinases, and therefore offered a more specific target for a particular chemotherapy response protein.

### 5.8.1. Production of Monoclonal Antibodies Specific for a CR Protein

Antibodies can be prepared by immunizing a suitable subject with a chemotherapy response protein or a fragment thereof as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see Current Protocols in Immunology, 1994, John Wiley & Sons, Inc., New York, N.Y.). Hybridoma cells producing a monoclonal antibody are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see, e.g., U.S. Pat. No. 5,914,112, which is incorporated herein by reference in its entirety).

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay (ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103, Academic Press, 1986). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against a chemotherapy response protein or a fragment thereof can be identified and isolated by screening a recombinant combinatorial immunoglobulin library (e.g., an antibody phage display library) with the chemotherapy response protein or the fragment. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.)

Humanized antibodies are antibody molecules from non-human species having one or more complementarity determining regions (CDRs) from the non-human species and a framework region from a human immunoglobulin molecule. (See e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323; antibodies against hepatitis B in Cole et al. (1991, Proc. Natl. Acad. Sci. USA 88:2869); as well as against viral antigens-respiratory syncitial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with a chemotherapy response protein.

Monoclonal antibodies directed against a chemotherapy response protein can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif., see, for example, U.S. Pat. No. 5,985,615) and Medarex, Inc. (Princeton, N.J.), can be engaged to provide human antibodies directed against a chemotherapy response protein or a fragment thereof using technology similar to that described above.

Completely human antibodies which recognize and bind a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

A pre-existing anti-chemotherapy response protein antibody can be used to isolate additional antigens of the chemotherapy response protein by standard techniques, such as affinity chromatography or immunoprecipitation for use as immunogens. Moreover, such an antibody can be used to detect the protein (e.g., in a cellular lysate or cell supernatant) in order to evaluate the abundance and pattern of expression of chemotherapy response protein. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### 5.8.2. Production of Polyclonal Anti-CR Protein Antibodies

The anti-chemotherapy response protein antibodies can be produced by immunization of a suitable animal, such as but are not limited to mouse, rabbit, and horse.

An immunogenic preparation comprising a chemotherapy response protein or a fragment thereof can be used to prepare antibodies by immunizing a suitable subject (e.g., rabbit, goat, mouse or other mammal). An appropriate immunogenic preparation can contain, for example, recombinantly expressed or chemically synthesized chemotherapy response protein peptide or polypeptide. The preparation can further include an adjuvant, such as Freund's complete or incomplete adjuvant, or similar immunostimulatory agent.

A fragment of a chemotherapy response protein suitable for use as an immunogen comprises at least a portion of the chemotherapy response protein that is 8 amino acids, more preferably 10 amino acids and more preferably still, 15 amino acids long.

The invention also provides chimeric or fusion chemotherapy response protein polypeptides for use as immunogens. As used herein, a “chimeric” or “fusion” chemotherapy response protein polypeptide comprises all or part of a chemotherapy response protein polypeptide operably linked to a heterologous polypeptide. Within the fusion chemotherapy response protein polypeptide, the term “operably linked” is intended to indicate that the chemotherapy response protein polypeptide and the heterologous polypeptide are fused in-frame to each other. The heterologous polypeptide can be fused to the N-terminus or C-terminus of the chemotherapy response protein polypeptide.

One useful fusion chemotherapy response protein polypeptide is a GST fusion chemotherapy response protein polypeptide in which the chemotherapy response protein polypeptide is fused to the C-terminus of GST sequences. Such fusion chemotherapy response protein polypeptides can facilitate the purification of a recombinant chemotherapy response protein polypeptide.

In another embodiment, the fusion chemotherapy response protein polypeptide contains a heterologous signal sequence at its N-terminus so that the chemotherapy response protein polypeptide can be secreted and purified to high homogeneity in order to produce high affinity antibodies. For example, the native signal sequence of an immunogen can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal and the protein A secretory signal (Pharmacia Biotech; Piscataway, N.J.).

In yet another embodiment, the fusion chemotherapy response protein polypeptide is an immunoglobulin fusion protein in which all or part of a chemotherapy response protein polypetide is fused to sequences derived from a member of the immunoglobulin protein family. The immunoglobulin fusion proteins can be used as immunogens to produce antibodies directed against the chemotherapy response protein polypetide in a subject.

Chimeric and fusion chemotherapy response protein polypeptide can be produced by standard recombinant DNA techniques. In one embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (e.g., Ausubel et al., supra). Moreover, many expression vectors are commercially available that already encode a fusion domain (e.g., a GST polypeptide). A nucleic acid encoding an immunogen can be cloned into such an expression vector such that the fusion domain is linked in-frame to the polypeptide.

The chemotherapy response protein immunogenic preparation is then used to immunize a suitable animal. Preferably, the animal is a specialized transgenic animal that can secret human antibody. Non-limiting examples include transgenic mouse strains which can be used to produce a polyclonal population of antibodies directed to a specific pathogen (Fishwild et al., 1996, Nature Biotechnology 14:845-851; Mendez et al., 1997, Nature Genetics 15:146-156). In one embodiment of the invention, transgenic mice that harbor the unrearranged human immunoglobulin genes are immunized with the target immunogens. After a vigorous immune response against the immunogenic preparation has been elicited in the mice, blood samples of the mice are collected and a purified preparation of human IgG molecules can be produced from the plasma or serum. Any method known in the art can be used to obtain the purified preparation of human IgG molecules, including but is not limited to affinity column chromatography using anti-human IgG antibodies bound to a suitable column matrix. Anti-human IgG antibodies can be obtained from any sources known in the art, e.g., from commercial sources such as Dako Corporation and ICN. The preparation of IgG molecules produced comprises a polyclonal population of IgG molecules that bind to the immunogen or immunogens at different degree of affinity. Preferably, a substantial fraction of the preparation contains IgG molecules specific to the immunogen or immunogens. Although polyclonal preparations of IgG molecules are described, it is understood that polyclonal preparations comprising any one type or any combination of different types of immunoglobulin molecules are also envisioned and are intended to be within the scope of the present invention.

A population of antibodies directed to a chemotherapy response protein can be produced from a phage display library. Polyclonal antibodies can be obtained by affinity screening of a phage display library having a sufficiently large and diverse population of specificities with a chemotherapy response protein or a fragment thereof. Examples of methods and reagents particularly amenable for use in generating and screening antibody display library can be found in, for example, U.S. Patent Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734. A phage display library permits selection of desired antibody or antibodies from a very large population of specificities. An additional advantage of a phage display library is that the nucleic acids encoding the selected antibodies can be obtained conveniently, thereby facilitating subsequent construction of expression vectors.

In other preferred embodiments, the population of antibodies directed to a chemotherapy response protein or a fragment thereof is produced by a method using the whole collection of selected displayed antibodies without clonal isolation of individual members as described in U.S. Pat. No. 6,057,098, which is incorporated by reference herein in its entirety. Polyclonal antibodies are obtained by affinity screening of a phage display library having a sufficiently large repertoire of specificities with, e.g., an antigenic molecule having multiple epitopes, preferably after enrichment of displayed library members that display multiple antibodies. The nucleic acids encoding the selected display antibodies are excised and amplified using suitable PCR primers. The nucleic acids can be purified by gel electrophoresis such that the full length nucleic acids are isolated. Each of the nucleic acids is then inserted into a suitable expression vector such that a population of expression vectors having different inserts is obtained. The population of expression vectors is then expressed in a suitable host.

### 5.8.3 Production of Peptides

A chemotherapy response protein-binding peptide or polypeptide or peptide or polypeptide of a chemotherapy response protein may be produced by recombinant DNA technology using techniques well known in the art. Thus, the polypeptide or peptide can be produced by expressing nucleic acid containing sequences encoding the polypeptide or peptide. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding chemotherapy response protein polypeptide sequences may be chemically synthesized using, for example, synthesizers. See, for example, the techniques described in “Oligonucleotide Synthesis”, 1984, Gait, M. J. ed., IRL Press, Oxford, which is incorporated herein by reference in its entirety.

### 5.9. Chemotherapeutic Drugs

The invention can be practiced with any known chemotherapeutic drugs, including but not limited to DNA damaging agents, anti-metabolites, anti-mitotic agents, or a combination of two or more of such known anti-cancer agents.

DNA damage agents cause chemical damage to DNA and/or RNA. DNA damage agents can disrupt DNA replication or cause the generation of nonsense DNA or RNA. DNA damaging agents include but are not limited to topoisomerase inhibitor, DNA binding agent, and ionizing radiation. A topoisomerase inhibitor that can be used in conjunction with the invention can be a topoisomerase I (Topo I) inhibitor, a topoisomerase II (Topo II) inhibitor, or a dual topoisomerase I and II inhibitor. A topo I inhibitor can be for example from any of the following classes of compounds: camptothecin analogue (e.g., karenitecin, aminocamptothecin, lurtotecan, topotecan, irinotecan, BAY 56-3722, rubitecan, GI14721, exatecan mesylate), rebeccamycin analogue, PNU 166148, rebeccamycin, TAS-103, camptothecin (e.g., camptothecin polyglutamate, camptothecin sodium), intoplicine, ecteinascidin 743, J-107088, pibenzimol. Examples of preferred topo I inhibitors include but are not limited to camptothecin, topotecan (hycaptamine), irinotecan (irinotecan hydrochloride), belotecan, or an analogue or derivative of any of the foregoing.

A topo II inhibitor that can be used in conjunction with the invention can be for example from any of the following classes of compounds: anthracycline antibiotics (e.g., carubicin, pirarubicin, daunorubicin citrate liposomal, daunomycin, 4-iodo-4-doxydoxorubicin, doxorubicin, n,n-dibenzyl daunomycin, morpholinodoxorubicin, aclacinomycin antibiotics, duborimycin, menogaril, nogalamycin, zorubicin, epirubicin, marcellomycin, detorubicin, annamycin, 7-cyanoquinocarcinol, deoxydoxorubicin, idarubicin, GPX-100, MEN-10755, valrubicin, KRN5500), epipodophyllotoxin compound (e.g., podophyllin, teniposide, etoposide, GL331, 2-ethylhydrazide), anthraquinone compound (e.g., ametantrone, bisantrene, mitoxantrone, anthraquinone), ciprofloxacin, acridine carboxamide, amonafide, anthrapyrazole antibiotics (e.g., teloxantrone, sedoxantrone trihydrochloride, piroxantrone, anthrapyrazole, losoxantrone), TAS-103, fostriecin, razoxane, XK469R, XK469, chloroquinoxaline sulfonamide, merbarone, intoplicine, elsamitrucin, CI-921, pyrazoloacridine, elliptinium, amsacrine. Examples of preferred topo II inhibitors include but are not limited to doxorubicin (Adriamycin), etoposide phosphate (etopofos), teniposide, sobuzoxane, or an analogue or derivative of any of the foregoing.

DNA binding agents that can be used in conjunction with the invention include but are not limited to a DNA groove binding agent, e.g., DNA minor groove binding agent; DNA crosslinking agent; intercalating agent; and DNA adduct forming agent. A DNA minor groove binding agent can be an anthracycline antibiotic, mitomycin antibiotic (e.g., porfiromycin, KW-2149, mitomycin B, mitomycin A, mitomycin C), chromomycin A3, carzelesin, actinomycin antibiotic (e.g., cactinomycin, dactinomycin, actinomycin F1), brostallicin, echinomycin, bizelesin, duocarmycin antibiotic (e.g., KW 2189), adozelesin, olivomycin antibiotic, plicamycin, zinostatin, distamycin, MS-247, ecteinascidin 743, amsacrine, anthramycin, and pibenzimol, or an analogue or derivative of any of the foregoing.

DNA crosslinking agents include but are not limited to antineoplastic alkylating agent, methoxsalen, mitomycin antibiotic, and psoralen. An antineoplastic alkylating agent can be a nitrosourea compound (e.g., cystemustine, tauromustine, semustine, PCNU, streptozocin, SarCNU, CGP-6809, carmustine, fotemustine, methylnitrosourea, nimustine, ranimustine, ethylnitrosourea, lomustine, chlorozotocin), mustard agent (e.g., nitrogen mustard compound, such as spiromustine, trofosfamide, chlorambucil, estramustine, 2,2,2-trichlorotriethylamine, prednimustine, novembichin, phenamet, glufosfamide, peptichemio, ifosfamide, defosfamide, nitrogen mustard, phenesterin, mannomustine, cyclophosphamide, melphalan, perfosfamide, mechlorethamine oxide hydrochloride, uracil mustard, bestrabucil, DHEA mustard, tallimustine, mafosfamide, aniline mustard, chlornaphazine; sulfur mustard compound, such as bischloroethylsulfide; mustard prodrug, such as TLK286 and ZD2767), ethylenimine compound (e.g., mitomycin antibiotic, ethylenimine, uredepa, thiotepa, diaziquone, hexamethylene bisacetamide, pentamethylmelamine, altretamine, carzinophilin, triaziquone, meturedepa, benzodepa, carboquone), alkylsulfonate compound (e.g., dimethylbusulfan, Yoshi-864, improsulfan, piposulfan, treosulfan, busulfan, hepsulfam), epoxide compound (e.g., anaxirone, mitolactol, dianhydrogalactitol, teroxirone), miscellaneous alkylating agent (e.g., ipomeanol, carzelesin, methylene dimethane sulfonate, mitobronitol, bizelesin, adozelesin, piperazinedione, VNP40101M, asaley, 6-hydroxymethylacylfulvene, E09, etoglucid, ecteinascidin 743, pipobroman), platinum compound (e.g., ZD0473, liposomal-cisplatin analogue, satraplatin, BBR 3464, spiroplatin, ormaplatin, cisplatin, oxaliplatin, carboplatin, lobaplatin, zeniplatin, iproplatin), triazene compound (e.g., imidazole mustard, CB10-277, mitozolomide, temozolomide, procarbazine, dacarbazine), picoline compound (e.g., penclomedine), or an analogue or derivative of any of the foregoing. Examples of preferred alkylating agents include but are not limited to cisplatin, dibromodulcitol, fotemustine, ifosfamide (ifosfamid), ranimustine (ranomustine), nedaplatin (latoplatin), bendamustine (bendamustine hydrochloride), eptaplatin, temozolomide (methazolastone), carboplatin, altretamine (hexamethylmelamine), prednimustine, oxaliplatin (oxalaplatinum), carmustine, thiotepa, leusulfon (busulfan), lobaplatin, cyclophosphamide, bisulfan, melphalan, and chlorambucil, or an analogue or derivative of any of the foregoing.

Intercalating agents can be an anthraquinone compound, bleomycin antibiotic, rebeccamycin analogue, acridine, acridine carboxamide, amonafide, rebeccamycin, anthrapyrazole antibiotic, echinomycin, psoralen, LU 79553, BW A773U, crisnatol mesylate, benzo(a)pyrene-7,8-diol-9,10-epoxide, acodazole, elliptinium, pixantrone, or an analogue or derivative of any of the foregoing.

DNA adduct forming agents include but are not limited to enediyne antitumor antibiotic (e.g., dynemicin A, esperamicin A1, zinostatin, dynemicin, calicheamicin gamma 1I), platinum compound, carmustine, tamoxifen (e.g., 4-hydroxy-tamoxifen), psoralen, pyrazine diazohydroxide, benzo(a)pyrene-7,8-diol-9,10-epoxide, or an analogue or derivative of any of the foregoing.

Anti-metabolites block the synthesis of nucleotides or deoxyribonucleotides, which are necessary for making DN, thereby preventing cells from replicating. Anti-metabolites include but are not limited to cytosine, arabinoside, floxuridine, 5-fluorouracil (5-FU), mercaptopurine, gemcitabine, hydroxyurea (HU), and methotrexate (MTX).

Anti-mitotic agents disrupt the development of the mitotic spindle thereby interfering with tumor cell proliferation. Anti-mitotic agents include but are not limited to Vinblastine, Vincristine, and Paclitaxel (Taxol). Anti-mitotic agents also include agents that target the enzymes that regulate mitosis, e.g., agents that target kinesin spindle protein (KSP), e.g., L-001000962-000Y.

### 5.10. Pharmaceutical Formulations and Routes of Administration

The compounds that can be used to modulate the expression of the chemotherapy response genes or the activity of their gene products can be administered to a patient at effective doses. Such an effective dose refers to that amount of the compound sufficient to result in the desired change in the expression or activity level of one or more CR genes and/or gene products thereof.

### 5.10.1. Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### 5.10.2. Formulations and Use

Pharmaceutical compositions for use in accordance with the present invention may be formulated in conventional manner using one or more pharmaceutically acceptable carriers or excipients.

Thus, the compounds and their pharmaceutically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### 5.10.3. Routes of Administration

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

### 5.10.4. Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound formulated in a compatible pharmaceutical carrier may also be prepared, placed in an appropriate container, and labeled for treatment of an indicated condition. Suitable conditions indicated on the label may include treatment of a disease such as one characterized by aberrant or excessive expression or activity of a chemotherapy response protein.

### 5.10.5. Combination Therapy

In a combination therapy, one or more compositions of the present invention, e.g., agent that reduces the level of expression and/or activity of one or more CR genes and/or gene products thereof, can be administered before, at the same time as, or after the administration of a chemotherapeutic agent. In one embodiment, the compositions of the invention are administered before the administration of a chemotherapeutic agent (i.e., the agent that modulates expression or activity of a chemotherapy response gene and/or encoded protein is for sequential or concurrent use with one or more the chemotherapeutic agent). In one embodiment, the composition of the invention and a chemotherapeutic agent are administered in a sequence and within a time interval such that the composition of the invention and a chemotherapeutic agent can act together to provide an increased benefit than if they were administered alone. In another embodiment, the composition of the invention and a chemotherapeutic agent are administered sufficiently close in time so as to provide the desired therapeutic outcome. The time intervals between the administration of the compositions of the invention and a chemotherapeutic agent can be determined by routine experiments that are familiar to one skilled person in the art. In one embodiment, a chemotherapeutic agent is given to the patient after the level of the chemotherapy response gene and/or encoded protein reaches a desirable threshold. The level of a chemotherapy response gene and/or encoded protein can be determined by using any techniques known in the art such as those described in Section 5.3., infra.

The composition of the invention and a chemotherapeutic agent can be administered simultaneously or separately, in any appropriate form and by any suitable route. In one embodiment, the composition of the invention and the chemotherapeutic agent are administered by different routes of administration. In an alternate embodiment, each is administered by the same route of administration. The composition of the invention and the chemotherapeutic agent can be administered at the same or different sites, e.g. arm and leg.

In various embodiments, such as those described above, the composition of the invention and a chemotherapeutic agent are administered less than 1 hour apart, at about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart, or no more than 1 week or 2 weeks or 1 month or 3 months apart. As used herein, the word about means within 10%. In other embodiments, the composition of the invention and a chemotherapeutic agent are administered 2 to 4 days apart, 4 to 6 days apart, 1 week apart, 1 to 2 weeks apart, 2 to 4 weeks apart, one month apart, 1 to 2 months apart, or 2 or more months apart. In preferred embodiments, the composition of the invention and a chemotherapeutic agent are administered in a time frame where both are still active. One skilled in the art would be able to determine such a time frame by determining the half life of each administered component. In separate or in the foregoing embodiments, the composition of the invention and a chemotherapeutic agent are administered less than 2 weeks, one month, six months, 1 year or 5 years apart.

In another embodiment, the compositions of the invention are administered at the same time or at the same patient visit, as the chemotherapeutic agent.

In still another embodiment, one or more of the compositions of the invention are administered both before and after the administration of a chemotherapeutic agent. Such administration can be beneficial especially when the chemotherapeutic agent has a longer half life than that of the one or more of the compositions of the invention used in the treatment.

In one embodiment, the chemotherapeutic agent is administered daily and the composition of the invention is administered once a week for the first 4 weeks, and then once every other week thereafter. In one embodiment, the chemotherapeutic agent is administered daily and the composition of the invention is administered once a week for the first 8 weeks, and then once every other week thereafter.

In certain embodiments, the composition of the invention and the chemotherapeutic agent are cyclically administered to a subject. Cycling therapy involves the administration of the composition of the invention for a period of time, followed by the administration of a chemotherapeutic agent for a period of time and repeating this sequential administration. Cycling therapy can reduce the development of resistance to one or more of the therapies, avoid or reduce the side effects of one of the therapies, and/or improve the efficacy of the treatment. In such embodiments, the invention contemplates the alternating administration of the composition of the invention followed by the administration of a chemotherapeutic agent 4 to 6 days later, preferable 2 to 4 days, later, more preferably 1 to 2 days later, wherein such a cycle may be repeated as many times as desired.

In certain embodiments, the composition of the invention and a chemotherapeutic agent are alternately administered in a cycle of less than 3 weeks, once every two weeks, once every 10 days or once every week. In a specific embodiment of the invention, one cycle can comprise the administration of a chemotherapeutic agent by infusion over 90 minutes every cycle, 1 hour every cycle, or 45 minutes every cycle. Each cycle can comprise at least 1 week of rest, at least 2 weeks of rest, at least 3 weeks of rest. In an embodiment, the number of cycles administered is from 1 to 12 cycles, more typically from 2 to 10 cycles, and more typically from 2 to 8 cycles.

It will be apparent to one skilled person in the art that any combination of different timing of the administration of the compositions of the invention and a chemotherapeutic agent can be used. For example, when the chemotherapeutic agent has a longer half life than that of the composition of the invention, it is preferable to administer the compositions of the invention before and after the administration of the chemotherapeutic agent.

The frequency or intervals of administration of the compositions of the invention depends on the desired level of the chemotherapy response gene and/or encoded protein, which can be determined by any of the techniques known in the art, e.g., those techniques described infra. The administration frequency of the compositions of the invention can be increased or decreased when the level of the chemotherapy response gene and/or encoded protein changes either higher or lower from the desired level.

### 5.11. Implementation Systems and Methods

The analytical methods of the present invention can preferably be implemented using a computer system, such as the computer system described in this section, according to the following programs and methods. Such a computer system can also preferably store and manipulate measured signals obtained in various experiments that can be used by a computer system implemented with the analytical methods of this invention. Accordingly, such computer systems are also considered part of the present invention.

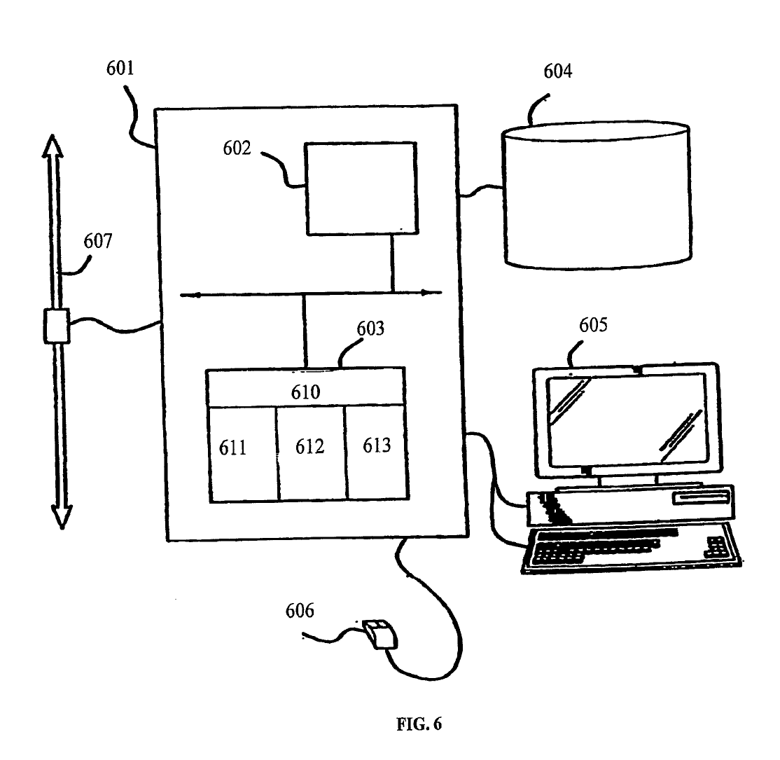

An exemplary computer system suitable from implementing the analytic methods of this invention is illustrated in FIG. 6. Computer system 601 is illustrated here as comprising internal components and as being linked to external components. The internal components of this computer system include one or more processor elements 602 interconnected with a main memory 603. For example, computer system 601 can be an Intel Pentium IV®-based processor of 2 GHZ or greater clock rate and with 256 MB or more main memory. In a preferred embodiment, computer system 601 is a cluster of a plurality of computers comprising a head “node” and eight sibling “nodes,” with each node having a central processing unit (“CPU”). In addition, the cluster also comprises at least 128 MB of random access memory (“RAM”) on the head node and at least 256 MB of RAM on each of the eight sibling nodes. Therefore, the computer systems of the present invention are not limited to those consisting of a single memory unit or a single processor unit.

The external components can include a mass storage 604. This mass storage can be one or more hard disks that are typically packaged together with the processor and memory. Such hard disks are typically of 10 GB or greater storage capacity and more preferably have at least 40 GB of storage capacity. For example, in a preferred embodiment, described above, wherein a computer system of the invention comprises several nodes, each node can have its own hard drive. The head node preferably has a hard drive with at least 10 GB of storage capacity whereas each sibling node preferably has a hard drive with at least 40 GB of storage capacity. A computer system of the invention can further comprise other mass storage units including, for example, one or more floppy drives, one more CD-ROM drives, one or more DVD drives or one or more DAT drives.

Other external components typically include a user interface device 605, which is most typically a monitor and a keyboard together with a graphical input device 606 such as a “mouse.” The computer system is also typically linked to a network link 607 which can be, e.g., part of a local area network (“LAN”) to other, local computer systems and/or part of a wide area network (“WAN”), such as the Internet, that is connected to other, remote computer systems. For example, in the preferred embodiment, discussed above, wherein the computer system comprises a plurality of nodes, each node is preferably connected to a network, preferably an NFS network, so that the nodes of the computer system communicate with each other and, optionally, with other computer systems by means of the network and can thereby share data and processing tasks with one another.

Loaded into memory during operation of such a computer system are several software components that are also shown schematically in FIG. 6. The software components comprise both software components that are standard in the art and components that are special to the present invention. These software components are typically stored on mass storage such as the hard drive 604, but can be stored on other computer readable media as well including, for example, one or more floppy disks, one or more CD-ROMs, one or more DVDs or one or more DATs. Software component 610 represents an operating system which is responsible for managing the computer system and its network interconnections. The operating system can be, for example, of the Microsoft Windows™ family such as Windows 95, Window 98, Windows NT, Windows 2000 or Windows XP. Alternatively, the operating software can be a Macintosh operating system, a UNIX operating system or a LINUX operating system. Software component 611 comprises common languages and functions that are preferably present in the system to assist programs implementing methods specific to the present invention. Languages that can be used to program the analytic methods of the invention include, for example, C and C++, FORTRAN, PERL, HTML, JAVA, and any of the UNIX or LINUX shell command languages such as C shell script language. The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

It will be clear to one skilled in the art that the computer system may comprise an outputting or displaying system for communicating a result from the analysis to an end user. In some embodiments, the outputting or display system comprises extenal component(s). It will be clear to one skilled in the art that outputting the result is not limited to outputting to linked external component(s), but may alternatively or additionally be outputting to internal component(s). It will also be clear to one skilled in the art that the claimed methods can, but need not be, computer-implemented, and that, for example, the displaying or outputting step can be done by, for example, by communicating to a person orally or in writing (e.g., in handwriting).

Software component 612 comprises any analytic methods of the present invention described supra, preferably programmed in a procedural language or symbolic package. For example, software component 612 preferably includes programs that cause the processor to implement steps of accepting a plurality of measured signals and storing the measured signals in the memory. For example, the computer system can accept measured signals that are manually entered by a user (e.g., by means of the user interface). More preferably, however, the programs cause the computer system to retrieve measured signals from a database. Such a database can be stored on a mass storage (e.g., a hard drive) or other computer readable medium and loaded into the memory of the computer, or the compendium can be accessed by the computer system by means of the network 607.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

### 5.12. Kits

The invention provides kits that are useful in determining chemotherapy responsiveness in a patient. The kits of the present invention comprise one or more probes and/or primers for one or more gene products or for each of at least 2, 5, 10, 20, or 30 gene products that are encoded by the respectively marker genes listed in Table 1 or functional equivalents of such genes, wherein the probes and/or primers are at least 50%, 75%, 80%, 85%, 90%, 95%, 97%, 98%, 99% or 100% of the total probes and/or primers in the kit. The probes of marker genes may be part of an array, or the biomarker(s) may be packaged separately and/or individually.

In one embodiment, the invention provides kits comprising probes that are immobilized at an addressable position on a substrate, e.g., in a microarray. In a particular embodiment, the invention provides such a microarray.

The kits of the present invention may also contain probes that can be used to detect protein products of the marker genes of the invention. In a specific embodiment, the invention provides a kit comprises a plurality of antibodies that specifically bind one or more, or a plurality of at least 5, 10, 20, or 30 proteins that are encoded by the respectively marker genes listed in Table 1 or functional equivalents of such genes, wherein the antibodies are at least 50%, 75%, 80%, 85%, 90%, 95%, 97%, 98%, 99% or 100% of the total antibodies in the kit. In accordance with this embodiment, the kit may comprise a set of antibodies or functional fragments or derivatives thereof (e.g., Fab, F(ab′)₂, Fv, or scFv fragments). In accordance with this embodiment, the kit may include antibodies, fragments or derivatives thereof (e.g., Fab, F(ab′)₂, Fv, or scFv fragments) that are specific for these proteins. In one embodiment, the antibodies may be detectably labeled.

The kits of the present invention may also include reagents such as buffers, or other reagents that can be used in obtaining the marker profile. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like.

In some embodiments of the invention, the kits of the present invention comprise a microarray. The microarray can be any of the microarrays described above, e.g., in Section 5.3.2, optionally in a sealed container. In one embodiment this microarray comprises a plurality of probe spots, wherein at least 20%, 40%, 60%, 80%, or 90% of the probe spots in the plurality of probe spots correspond to marker genes listed in Table 1.

In still other embodiments, the kits of the invention may further comprise a computer program product for use in conjunction with a computer system, wherein the computer program product comprises a computer readable storage medium and a computer program mechanism embedded therein. In such kits, the computer program mechanism comprises instructions for prediction of prognosis using a marker profile obtained with the reagents of the kits.

In still other embodiments, the kits of the present invention comprise a computer having a central processing unit and a memory coupled to the central processing unit. The memory stores instructions for prediction of prognosis using a marker profile obtained with the reagents of the kits.

### 6. EXAMPLES

The following examples are presented by way of illustration of the present invention, and are not intended to limit the present invention in any way.

A 311 cohort samples were collected from breast cancer patients. See van de Vijver et al., 2002, A gene-expression signature as a predictor of survival in breast cancer. N Engl J Med. 347(25):1999-2009. Microarrays containing approximately 25,000 human gene sequences (Hu25K microarrays) were used for this study. Sequences for microarrays were selected from RefSeq (a collection of non-redundant mRNA sequences, located on the Internet at nlm.nih.gov/LocusLink/refseq.html) and Phil Green EST contigs, which is a collection of EST contigs assembled by Dr. Phil Green et al at the University of Washington (Ewing and Green, Nat. Genet. 25(2):232-4 (2000)), available on the Internet at phrap.org/est_assembly/index.html. Each mRNA or EST contig was represented on Hu25K microarray by a single 60 mer oligonucleotide essentially as described in Hughes et al., Nature Biotech. 19(4):342-347 and in International Publication WO 01/06013, published Jan. 25, 2001, and in International Publication WO 01/05935, published Jan. 25, 2001, except that the rules for oligo screening were modified to remove oligonucleotides with more than 30% C or with 6 or more contiguous C residues.

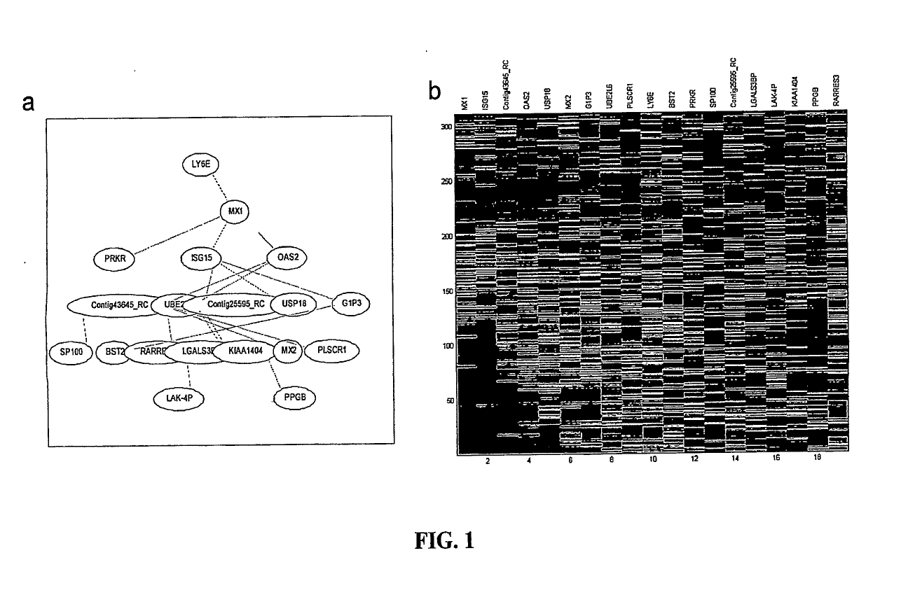

Using the 311 NKI breast cancer cohort sample data and a “nearest neighbor” method, a total of 122 hubs/networks were constructed (magnitude of correlation coefficient for connected genes>0.5). Among the 311 patients, 110 patients received chemotherapy of either 5-fluorouracil or CMF combination (consisting of cyclophosphamide, methotrexate, and 5-fluorouracil). FIG. 1 shows one example of such a hub.

FIG. 1. (a) A network (hub #34) enriched for interferon stimulated genes (ISG). (b) The hub genes are highly co-regulated in breast cancer data where the network was derived from. Each row represents a sample, each column represents one gene. A darker shade, which was magenta in the original depiction of FIG. 1b, represents up-regulation; and a lighter shade, which was cyan in the original depiction of FIG. 1b, represents down regulation.

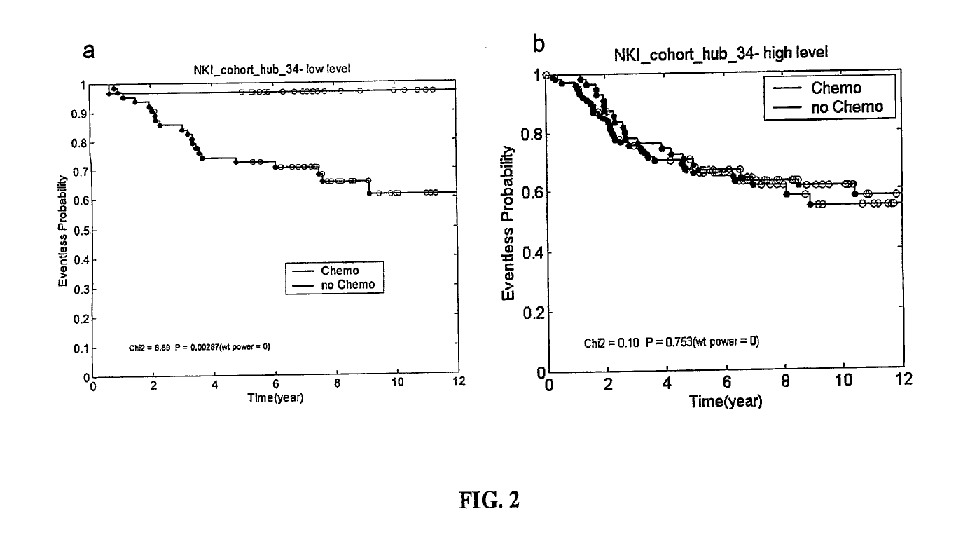

As a second step, the hub expression level in each breast cancer sample was computed by averaging over genes in each hub. Hubs whose expression levels were related to chemotherapy sensitivity were searched for by first dividing samples into two populations according to the hub expression level. Within each population, the treatment effect was examined by checking whether the metastasis rate was affected by the chemotherapy. Specifically a log-rank-test was performed on the metastasis free probability as a function of time for patients with treatment vs. no treatment. When this search was performed over all 311 samples, there were only two hubs with log-rank-test P-value<0.01. Among the two, the most significant one was a hub (#34) enriched for interferon stimulated genes (ISGs) (FIGS. 1 and 2), with a P-value of 0.3%.

Since breast cancer expression patterns are very different between the estrogen receptor positive (ER+) patients and negative (ER−) patients, a search was also performed over ER+ (239 samples) and ER− patients (72 samples), respectively. A total of 7 hubs with log-rank-test P-value<0.01 were identified. Again, the ISG hub is among the 7 and with the most significant P-value (0.3%). Given the ISG hub was the most promising hub for “predicting” the chemo-sensitivity, all 122 constructed hubs were re-examined and another hub (hub #88) that was also enriched for ISGs was identified. The log-rank-test P-value for this hub was 2%, barely missed the selection criteria.

FIG. 2. The expression level of interferon stimulated genes (ISGs) is related to chemotherapy (CMF) sensitivity in breast cancer patients. (a) Patients with low expression of ISGs show great chemotherapy sensitivity as indicated by the Kaplan-Meier plot of metastasis-free probability between patients received the treatment (red) vs. no treatment (blue). At 10 years after diagnosis of cancer, the treatment boosted the metastasis-free probability from 60% to ˜95%(log-rank-test P-value 0.3%). (b) Patients with high expression of ISGs show no chemo-therapy sensitivity. There was essentially no difference in metastasis-free probability between patients with and without chemotherapy (P=75%).

Thirdly, these 9 hubs (including hub #88 which just missed the threshold) were tested in an ex-vivo ovarian cancer data set. 50 ovarian cancer samples were plated ex-vivo and treated by a panel of 19 anticancer drugs, including Paclitaxel, carboplatin, etoposide, and 5-FU. The tumor cell growth inhibition for each drug treatment was measured and samples were categorized into 3 classes for each drug: EDR (extreme drug resistance), LDR (low drug resistance) and in between. The 50 ovarian cancer samples pre-dose of drugs were profiled against the pool of all samples. The expression levels of hub genes were tested by their correlation to the drug resistance categories.

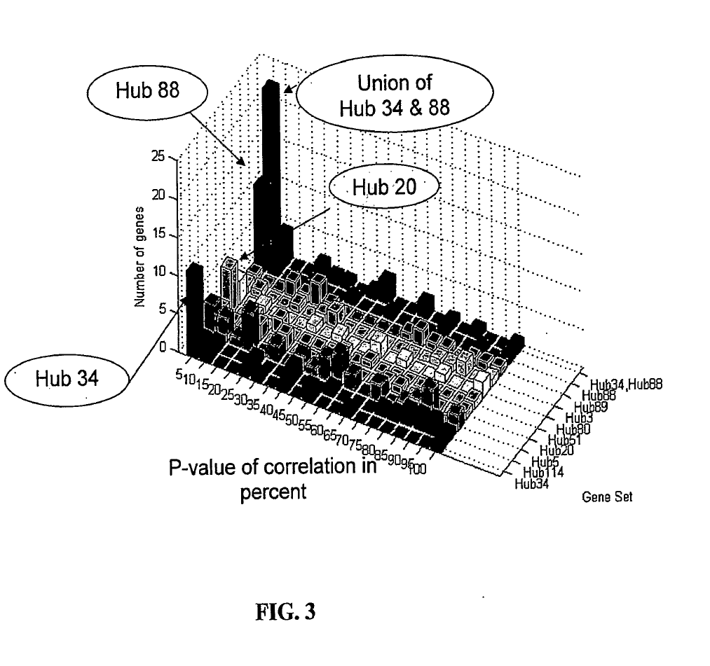

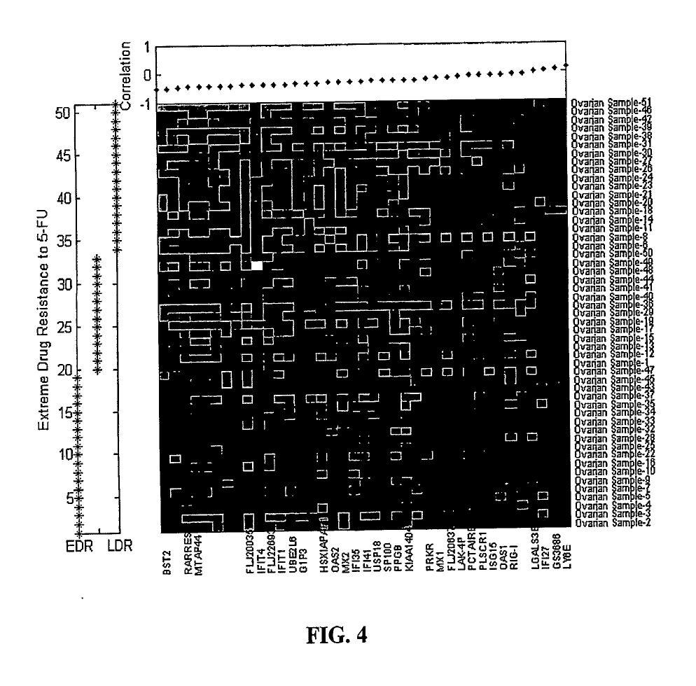

Among the 9 hubs tested, only 3 hubs (#20, #34 and #88, two of which were enriched for ISGs) had significant fraction of members correlated (P-value of correlation<5%) to the growth inhibition by 5-FU (FIG. 3). The gene expression pattern of the two ISG hubs (see Table 1 for gene list) in ovarian cancer is shown in FIG. 4. As can be seen from this plot, low drug resistance mostly corresponded to the low expression of ISGs, and the extreme drug resistance mostly corresponded to high expression level of ISGs, agree well with the clinical breast cancer observation.

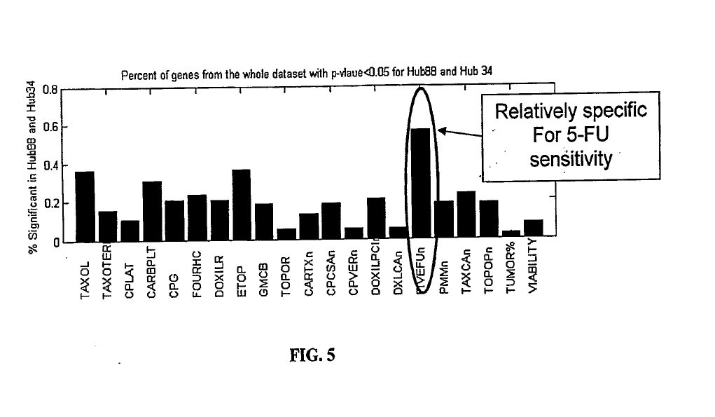

Finally, the specificity of ISG pathway reporting on the Paclitaxel, carboplatin, etoposide, and 5-FU sensitivity was examined. The correlation between expression level and drug resistance for all 19 anti-cancer drugs was calculated. As shown in FIG. 5, the number of genes correlated with drug resistance was above about 30% for Paclitaxel, carboplatin, etoposide, and 5-FU, indicating ISGs are relatively specific for reporting resistance to these drugs.

FIG. 3. Bar chart of number of genes in each P-value bin for 9 hubs. P-value was based on the correlation coefficient between gene expression level and 5-FUdrug resistance category in ovarian ex-vivo experiment. 3 hubs (#20, 34 & 88) had significant fraction of members whose base-line expression level correlated with the drug resistance (with P-value of correlation<5%). Two of the 3 hubs (#34 & 88) belong to ISG pathway.

FIG. 4. Expression of ISGs and their relation with drug resistance in ex-vivo ovarian samples. Left panel: category of 5-FU drug resistance measured by growth inhibition. EDR stands for extreme drug resistance, LDR stands for low drug resistance. The remaining category stands for intermediate. Heatmap: expression of ISGs from hub 34 & hub 88. Each row represents a sample, each column represents one gene. A darker shade, which was magenta in the original heatmap of FIG. 4, represents up-regulation; and a lighter shade, which was cyan in the original heatmap of FIG. 4, represents down regulation. For LDR samples, ISGs are mostly under-expressed compared to the average, whereas for EDR samples, the ISG levels are relatively higher. Top panel: correlation of expression level to drug resistance.

FIG. 5. Fraction of interferon-stimulated-genes (ISGs) correlated with drug resistance in ex-vivo ovarian cancer samples treated with a panel of anti-cancer drugs. The ISGs are relatively specific in reporting the 5-FU drug sensitivity.

In summary, a set of markers including many interferon-stimulated-genes are identified that correlates with chemotherapy sensitivity to Paclitaxel, carboplatin, etoposide, and 5-FU in both clinical and ex-vivo model systems. This demonstrates the utility of combining pathway analysis and model systems to help predict response to chemotherapy.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method for predicting the responsiveness of a mammalian patient having a cancer to a chemotherapy regimen, comprising:
predicting said mammalian patient (a) as responsive to said chemotherapy regimen, if expression and/or activity of one or more gene products in a cell sample taken from said mammalian patient is not up-regulated relative to a reference population of individuals of the same species as said mammalian patient; or (b) as non-responsive to said chemotherapy regimen, if expression and/or activity of said one or more gene products is up-regulated relative to said reference population of individuals, wherein said one or more gene products comprise respectively products of one or more different genes selected from the group consisting of genes corresponding to SEQ ID NOs:1-39 or respective functional equivalents thereof.

2. The method of claim 1, further comprising:
determining, prior to said predicting step, whether expression and/or activity of said one or more gene products is up-regulated as relative to said reference population of individuals.

3. The method of claim 2, wherein said determining step is carried out by a method comprising:
determining one or more chemotherapy response scores (CR scores) based on measurements of said one or more gene products in said cell sample, wherein said one or more CR scores indicate whether expression and/or activity of said one or more gene products is up-regulated as compared to individuals in said reference population.

4. The method of claim 3, comprising:
determining a CR score that is an average of said measurements of said one or more gene products, wherein said mammalian patient is predicted as responsive if said average is equal to a predetermined threshold value or as non-responsive if said average is greater than said predetermined threshold value.

5. The method of claim 3, comprising:
determining a first CR score that is a first measurement of a gene product of a gene having the greatest expressive range among a first subset of said one or more different genes, wherein said first subset is selected from the group consisting of genes having SEQ ID NOs:1-19 or
determining a second CR score that is a second measurement of a gene product of a gene having the greates expressive range among a second subset of said one or more different genes, wherein said second subset is selected from the group consisting of genes having SEQ ID NOs:20-39, wherein said mammalian patient is predicted as responsive if said first or second measurement is less or equal to a predetermined threshold value or as non-responsive if said first or second measurement is greater than said predetermined threshold value.

6. The method of claim 3, wherein said step of determining one or more CR scores is carried out by a method comprising:
(a1) comparing a marker profile comprising said measurements of said one or more gene products with a responsive template and/or a non-responsive template, wherein said responsive template comprises measurements of said one or more gene products representative of measurements of said one or more genes products in a plurality of mammalian patients being responsive to said chemotherapy regimen, and said non-responsive template comprises measurements of said one or more gene products representative of measurements of said plurality of genes products in a plurality of mammalian patients being non-responsive to said chemotherapy regimen; and
(a2) determining a first degree of similarity between said marker profile and said responsive template and/or a second degree of similarity between said marker profile and said non-responsive template, wherein said first and second degrees of similarity are said one or more CR scores, and wherein said mammalian patient is (b1) predicted to be responsive if said first degree of similarity is greater than said second degree of similarity or if said first degree of similarity is greater than a predetermined threshold or (b2) predicted to be non-responsive if said first degree of similarity is no greater than said second degree of similarity or if said second degree of similarity is no greater than said predetermined threshold.

7. The method of claim 6, wherein said first or second degree of similarity is represented by a correlation coefficient between said marker profile and said respective template.

8. The method of claim 6, wherein the measurement of each gene product in said responsive template is an average of the measurements of said gene product in a plurality of responsive mammalian patients, and wherein the measurement of each gene product in said non-responsive template is an average of the measurements of said gene product in a plurality of non-responsive mammalian patients.

9. The method of claim 3, wherein said step of determining one or more CR scores is carried out by a method comprising using a chemotherapy response classifier selected from the group consisting of an artificial neural network (ANN) classifier and a support vector machine (SVM) classifier, wherein said chemotherapy response classifier receives an input comprising a marker profile comprising said measurements of said one or more gene products and provides an output comprising said one or more CR scores.

10. The method of claim 9, wherein said chemotherapy response classifier is trained with training data from a plurality of training cancer patients, wherein said training data comprise for each patient of said plurality of training cancer patients (i) a training marker profile comprising measurements of said plurality of gene products in a cell sample taken from said training patient; and (ii) data indicating whether said training patient is responsive to said treatment regimen.

11. The method of claim 3, comprising determining one or more CR scores that indicates in which percentile said measurements of said one or more gene products fall in the said reference population of individuals, wherein said patient is predicted to be non-responsive if said one or more CR scores indicate that said measurements of said one or more gene products fall in the Y1 percentile in said reference population, wherein Y1 percentile=60 percentile, 70 percentile, 80 percentile, or 90 percentile, or is predicted to be responsive if said one or more CR scores indicate that said measurements of said one or more gene products fall in the Y2 percentile in said reference population, wherein Y2 percentile=10 percentile, 20 percentile, 30 percentile, or 40 percentile,

12. The method of claim 1, wherein said measurements of one or more gene products are measurements of abundance levels of gene transcripts.

13. The method of claim 1, wherein said measurements of one or more gene products are measurements of abundance levels of proteins.

14. The method of claim 1, wherein said chemotherapy regimen comprises administration of a chemotherapy drug selected from the group consisting of 5-fluorouracil, CMF combination consisting of cyclophosphamide, methotrexate, and 5-fluorouracil, paclitaxel, etoposide, and carboplatin.

15. The method of claim 1, wherein said one or more gene products are respectively products of said one or more different genes selected from the group consisting of genes having SEQ ID NOs:1-39.

16. The method of claim 1, wherein said one or more gene products are of at least N or are all of said one or more different genes selected from the group consisting of genes having SEQ ID NOs:1-39, wherein N=2, 3, 4, 5, 10, 15, 20, 25, 30, or 35.

17. The method of claim 1, wherein said one or more gene products are of at least N, or are all of said one or more different genes selected from the group consisting of genes having SEQ ID NOs:1-19. wherein N=2, 3, 4, 5, 10, or 15.

18. The method of claim 1, wherein said one or more gene products are of at least N, or are all of said one or more different genes selected from the group consisting of genes having SEQ ID NOs:20-39. wherein N=2, 3, 4, 5, 10, or 15.

19. The method of claim 16, wherein said one or more gene products comprises gene products of (i) at least N, or are all of said one or more different genes selected from the group consisting of genes having SEQ ID NOs:1-19, wherein N=2, 3, 4, 5, 10. or 15 and (ii) at least M, or are all of said one or more different genes selected from the group consisting of genes having SEQ ID NOs:20-39, wherein M=2, 3, 4, 5, 10, or 15.

20. The method of claim 1, wherein said chemotherapy regimen is an adjuvant chemotherapy regimen, and wherein a prediction of a patient as responsive to said chemotherapy regimen indicates non-occurrence of metastases or survival within a first predetermined period of time after initial diagnosis in said patient treated with said chemotherapy regimen, and wherein a prediction of a patient as non-responsive to said chemotherapy regimen indicates occurrence of metastases or non-survival within a second predetermined period of time in said patient treated with said chemotherapy regimen.

21. The method of claim 1, wherein said chemotherapy regimen is a primary chemotherapy regimen, and a prediction of a patient as responsive to said chemotherapy regimen indicates (i) a reduction in tumor size or number of cancer cells and/or (ii) non-occurrence of metastases or survival within a first predetermined period of time after initial diagnosis in said patient treated with said chemotherapy regimen, and wherein a prediction as responsive to said chemotherapy regimen indicates (iii) a lack of reduction in tumor size or number of cancer cells and/or (iv) occurrence of metastases or non-survival within a second predetermined period of time in said patient treated with said chemotherapy regimen.

22. The method of claim 20 or 21, wherein said first period of time and said second periods of time are the same, and are each 3, 5, 7, 10, or 12 years.

23. The method of claim 1, wherein said patient has been determined to have a poor prognosis, wherein a poor prognosis indicates occurrence of metastases or non-survival within a third predetermined period of time in said patient untreated with any chemotherapy for said cancer.

24. The method of claim 1, wherein said measurement of each said gene product is a relative eve of said gene product in said cell sample versus level of said gene product in a reference sample, represented as a log ratio.

25. The method of claim 24, wherein said reference sample is selected from the group consisting of a sample comprising a pool of cancer cells obtained from a plurality of patients having said cancer, a sample of cells of a non-cancerous cell line of cells of the same type of tissue as said cancer, and a sample of cells of a cell line of said cancer.

26. The method of claim 1, wherein said patient is a human patient.

27. The method of claim 1, wherein said cancer is breast cancer.

28. The method of claim 1, wherein said cancer is ovarian cancer.

29. A method for assigning a treatment regimen for a patient having a cancer, comprising
(i) predicting whether said patient is responsive or non-responsive to a chemotherapy regimen using the method of claim 1; and
(ii) if said patient is determined to be responsive to said chemotherapy regimen, assigning said patient a treatment regimen that comprises said chemotherapy regimen; or if said patient is determined to be non-responsive to said chemotherapy regimen, assigning said patient (ii1) a treatment regimen that does not comprise said chemotherapy regimen or (ii2) a treatment regiment comprising (A) said chemotherapy regimen and (B) one or more agents that reduce the expression and/or activity level of said one more gene products.

30. A method for enrolling a plurality of cancer patients for a clinical trial a chemotherapy regimen, comprising (i) determining whether each patient in said plurality is responsive or non-responsive to said chemotherapy regimen using the method of claim 1; and (ii) assigning each patient who is predicted to be responsive to one patient group and each patient who is predicted to be non-responsive to another patient group, at least one of said patient group being enrolled in said clinical trial.

31. The method of claim 1, wherein said method is a computer implemented method.

32. A computer system comprising
a processor, and
a memory coupled to said processor and encoding one or more programs,
wherein said one or more programs cause the processor to carry out the method of claim 1.

33. A computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method of claim 1.

34. The method of claim 1, further comprising obtaining said measurements of said one or more gene products by a method comprising measuring said plurality of gene products of said cell sample taken from said patient.

35. The method of claim 11, further comprising obtaining measurement of abundance level of each said gene transcript by a method comprising contacting a positionally-addressable microarray with nucleic acids from said cell sample or nucleic acids derived therefrom under hybridization conditions, and detecting the amount of hybridization that occurs, said microarray comprising one or more polynucleotide probes complementary to a hybridizable sequence of each said gene transcript or a nucleic acid derived thereof.

36. The method of claim 11, further comprising obtaining measurement of abundance level of each said gene transcript by a method comprising measuring the transcript level of said gene using quantitative reverse transcriptase PCT (qRT-PCR).

37. A method for treating a patient having a cancer, comprising administering to said patient (a) one or more agents that is capable of reducing the expression and/or activity of one or more different genes selected from the group consisting of genes having SEQ ID NOs:1-39 or respective functional equivalents thereof and/or their encoded proteins, and (b) a chemotherapy regimen, wherein said patient is predicted to be non-responsive to said chemotherapy regimen as a result of overexpression of said one or more different genes.

38. The method of claim 37, wherein said one or more different genes consist of at least N or all of the different genes selected from the group consisting of genes having SEQ ID NOs:1-39, wherein N=2, 3, 4, 5, 10, 15, 20, 25, 30, or 35.

39. The method of claim 38, wherein said one or more different genes consist of at least N or all of the different genes selected from the group consisting of genes having SEQ ID NOs:1-19, wherein N=2, 3, 4, 5, 10, or 15.

40. The method of claim 38, wherein said one or more different genes consist of at least N or all of the different genes selected from the group consisting of genes having SEQ ID NOs:20-39, wherein N=2, 3, 4, 5, 10 or 15.

41. The method of claim 38, wherein said one or more different gene are of (i) at least N or all of the different genes selected from the group consisting of genes having SEQ ID NOs:1-19, wherein N=2, 3, 4, 5, 10, or 15; and (ii) at least K or all of the different genes selected from the group consisting of genes having SEQ ID NOs:20-39, wherein K=2, 3, 4, 5, 10, or 15.

42. The method of claim 37, wherein said one or more agents comprise a substance selected from the group consisting of siRNA, antis oleic acid, ribozyme, and triple helix forming nucleic acid, each being capable of reducing the expression of one or more c f said one or more different genes.

43. The method of claim 37, wherein said one or more agents comprise a substance selected from the group consisting of antibody, peptide, and small molecule, each is capable of reducing the activity of one or more of proteins encoded by said one or more different genes.

44. The method of claim 42, wherein said one or more agents comprise an siRNA targeting said one or more different genes.

45. The method of claim 44, wherein said one or more different genes consist of at least L different genes, wherein L=2, 3, 4, 5, 10, or 15.

46. The method of claim 37, further comprising determining a transcript level of each of said one or more different genes.

47. The method of claim 46, wherein said determining each said transcript level is carried out by a method comprising measuring the transcript level of said gene using one or more polynucleotide probes, each of said one or more polynucleotide probes comprising a nucleotide sequence complementary to a hybridizable sequence in said transcript of said gene or a nucleic acid derived thereof.

48. The method of claim 47, wherein said one or more polynucleotide probes are polynucleotide probes on a microarray.

49. The method of claim 48, wherein said determining each said transcript level is carried out by a method comprising measuring the transcript level of said gene using quantitative reverse transcriptase PCT (qRT-PCR).

50. The method of claim 37, wherein said chemotherapy regimen comprises administering a chemotherapy drug selected from the group consisting of 5-fluorouracil, CMF combination consisting of cyclophosphamide, methotrexate, and 5-fluorouracil, paclitaxel, etoposide, carboplatin.

51. The method of claim 37, wherein said patient is a human patient.

52. The method of claim 37, wherein said cancer is breast cancer.

53. The method of claim 37, wherein said cancer is ovarian cancer.

54. A method for modulating sensitivity of a cell to a chemotherapeutic drug, comprising contacting said cell with one or more agents, said one or more agents being capable of reducing the expression and/or activity of one or more different genes selected from the group consisting of genes having SEQ ID NOs:1-39 or respective functional equivalents thereof and/or the their encoded proteins.

55. A method for modulating growth of a cell, comprising contacting said cell with (a) one or more agents, said one or more agents being capable of reducing the expression and/or activity of one or more different genes selected from the group consisting of genes having SEQ ID NOs:1-39 or respective functional equivalents thereof and/or the their encoded proteins; and (b) a sufficient amount of a chemotherapeutic drug.

56. The method of any one of claims 54 to 55, wherein said one or more different genes consist of at least N or all of the different genes selected from the group consisting of genes having SEQ ID NOs:1-39, wherein N=2, 3, 4, 5, 10, 15, 20, 25, 30, or 35.

57. The method of claim 56, wherein said one or more different genes consist of at least N or all of the different genes selected from the group consisting of genes having SEQ ID NOs:1-19, wherein N=2, 3, 4, 5, 10, or 15.

58. The method of claim 56, wherein said one or more different genes consist of at least N or all of the different genes selected from the group consisting of genes having SEQ ID NOs:20-39, wherein N=2, 3, 4, 5, 10, or 15.

59. The method of claim 56, wherein said one or more different gene are of (i) at least N or all of the different genes selected from the group consisting of genes having SEQ ID NOs:1-19, wherein N=2, 3, 4, 5, 10, or 15; and (ii) at least K or all of the different genes selected from the group consisting of genes having SEQ ID NOs:20-39, wherein K=2, 3, 4, 5, 10, or 15.

60. The method of claim 54 or 55, wherein said one or more agents comprise a substance selected from the group consisting of siRNA, antisense nucleic acid, ribozyme, and triple helix forming nucleic acid, each is capable of reducing the expression of one or more of said one or more different genes.

61. The method of claim 54 or 55, wherein said one or more agents comprise a substance selected from the group consisting of antibody, peptide, and small molecule, each is capable of reducing the activity of one or more of proteins encoded by said one or more different genes.

62. The method of claim 60, wherein said one or more agents comprise an siRNA targeting said one or more different genes.

63. The method of claim 62, wherein said one or more different genes consist of at least L different genes, wherein L=2, 3, 4, 5, 10, or 15.

64. The method of claim 54 or 55, further comprising determining a transcript level of each of said one or more different genes.

65. The method of claim 64, wherein said determining each said transcript level is carried out by a method comprising measuring the transcript level of said gene using one or more poly:nucleotide probes, each of said one or more polynucleotide probes comprising a nucleotide sequence complementary to a hybridizable sequence in said transcript of said gene or a nucleic acid derived thereof.

66. The method of claim 65, wherein said one or more polynucleotide probes are polynucleotide probes on a microarray.

67. The method of claim 65, wherein said determining each said transcript level is carried out by a method comprising measuring the transcript level of said gene using quantitative reverse transcriptase PCT (qRT-PCR).

68. The method of claim 54 or 55, wherein said chemotherapeutic drug is selected from the group consisting of 5-fluorouracil, CMF combination consisting of cyclophosphamide, methotrexate, and 5-fluorouracil, paclitaxel, etoposide, carboplatin.

69. The method of claim 54 or 55, wherein said cell is a human cell.

70. The method of claim 54 or 55, wherein said cell is a breast cancer cell.

71. The method of claim 54 or 55, wherein said cell is ovarian cancer cell.

72. A method of identifying an agent that is capable of modulating sensitivity of a cell to the growth inhibitory effect of a chemotherapeutic drug, said method comprising comparing a first growth inhibitory effect of said chemotherapeutic drug on cells expressing said gene in the presence of a candidate agent with a second growth inhibitory effect of said chemotherapeutic drug on cells expressing said gene in the absence of said agent, wherein said agent is capable of reducing the expression and/or activity of a gene selected from the group consisting of genes having SEQ ID NOs:1-39 or respective functional equivalents thereof and/or its encoded protein, wherein a difference in said first inhibitory effect and said second growth inhibitory effect identities said agent as capable of modulating sensitivity of said cell to the growth inhibitory effect of said chemotherapeutic drug.

73. The method of claim 72, further comprising:
(a) contacting a first cell expressing said gene with said chemotherapeutic drug in the presence of said agent and measuring said first growth inhibitory effect;
(b) contacting a second cell expressing said gene with said chemotherapeutic drug in the absence of said agent and measuring said second growth inhibitory effect.

74. The method of claim 72, wherein said agent comprises a substance selected from the group consisting of siRNA, antisense nucleic acid, ribozyme, and triple helix forming nucleic acid, each reducing the expression of said genes.

75. The method of claim 72, wherein said one or more agents comprise a substance selected from the group consisting of antibody, peptide, and small molecule, each reducing the activity of one or more of proteins encoded by said one or more different genes in said patient.

76. The method of claim 72, wherein said chemotherapy regimen comprises administering a chemotherapy drug selected from the group consisting of 5-fluorouracil, CMF combination consisting of cyclophosphamide, methotrexate, and 5-fluorouracil, paclitaxel, etoposide, carboplatin.

77. The method of claim 72, wherein said cell is a breast cancer cell.

78. The method of claim 72, wherein said cell is ovarian cancer cell.

79. A microarray comprising for each of one or more different genes selected from the group consisting of genes having SEQ ID NOs:1-39 or respective functional equivalents thereof, one or more polynucleotide probes complementary and hybridizable to a sequence in said gene, wherein polynucleotide probes complementary and hybridizable to said genes constitute at least 50%, 60%, 70%, 80% or 90% of the probes on said microarray.

80. The method of claim 79, wherein said one or more different genes consist of at least N or all of the different genes selected from the group consisting of genes having SEQ ID NOs:1-39, wherein N=2, 3, 4, 5, 10, 15, 20, 25, 30, or 35.

81. The method of claim 79, wherein said one or more different genes consist of at least N or all of the genes selected from the group consisting of genes having SEQ ID NOs:1-19, wherein N=2, 3, 4, 5, 10, 15.

82. The method of claim 79, wherein said one or more different genes consist of at least N or all of the different genes selected from the group consisting of genes having SEQ ID NOs:20-39, wherein N=2, 3, 4, 5, 10, or 15.

83. The method of claim 79, wherein said one or more different gene are of (i) at least Nor all of the different genes selected from the group consisting of genes having SEQ ID NOs:1-19, wherein N=2, 3, 4, 5, 10, or 15; and (ii) at least N or all of the different genes selected from the group consisting of genes having SEQ ID NOs:20-39, wherein N=2, 3, 4, 5, 10, or 15.

84. The method of claim 34, wherein said method is carried out in vivo.

85. The method of claim 34, wherein said method is carried out in vitro.


SEQUENCE LISTING (39 sequences)

SEQ ID NO: 1 - DNA, 1145 bp, Homo sapiens
gctccggccagccgcggtccagagcgcgcgaggttcggggagctccgccaggctgctggtacctgcgtccgcccggcgagcaggacaggctgctttggtttgtgacctccaggcaggacggccatcctctccagaatgaagatcttcttgccagtgctgctggctgcccttctgggtgtggagcgagccagctcgctgatgtgcttctcctgcttgaaccagaagagcaatctgtactgcctgaagccgaccatctgctccgaccaggacaactactgcgtgactgtgtctgctagtgccggcattgggaatctcgtgacatttggccacagcctgagcaagacctgttccccggcctgccccatcccagaaggcgtcaatgttggtgtggcttccatgggcatcagctgctgccagagctttctgtgcaatttcagtgcggccgatggcgggctgcgggcaagcgtcaccctgctgggtgccgggctgctgctgagcctgctgccggccctgctgcggtttggcccctgaccgcccagaccctgtcccccgatcccccagctcaggaaggaaagcccagccctttctggatcccacagtgtatgggagcccctgactcctcacgtgcctgatctgtgcccttggtcccaggtcaggcccaccccctgcacctccacctgccccagcccctgcctctgcccaagtgggccagctgccctcacttctggggtggatgatgtgaccttccttgggggactgcggaagggacgagggttccctggagtcttacggtccaacatcagaccaagtcccatggacatgctgacagggtccccagggagaccgtgtcagtagggatgtgtgcctggctgtgtacgtgggtgtgcagtgcacgtgagagcacgtggcggcttctgggggccatgtttggggagggaggtgtgccagcagcctggagagcctcagtccctgtagccccctgccctggcacagctgcatgcacttcaagggcagcctttgggggttggggtttctgccacttccgggtctaggccctgcccaaatccagccagtcctgccccagcccacccccacattggagccctcctgctgctttggtgcctcaaataaatacagatgtcccc

SEQ ID NO: 2 - DNA, 3579 bp, Homo sapiens
ctgaggcccacgcagggcctagggtgggaagatggcaggtgggggcggcgacctgagcaccaggaggctgaatgaatgtatttcaccagtagcaaatgagatgaaccatcttcctgcacacagccacgatttgcaaaggatgttcacggaagaccagggtgtagatgacaggctgctctatgacattgtattcaagcacttcaaaagaaataaggtggagatttcaaatgcaataaaaaagacatttccattcctcgagggcctccgtgatcgtgatctcatcacaaataaaatgtttgaagattctcaagattcttgtagaaacctggtccctgtacagagagtggtgtacaatgttcttagtgaactggagaagacatttaacctgccagttctggaagcactgttcagcgatgtcaacatgcaggaataccccgatttaattcacatttataaaggctttgaaaatgtaatccatgacaaattgcctctccaagaaagtgaagaagaagagagggaggagaggtctggcctccaactaagtcttgaacaaggaactggtgaaaactcttttcgaagcctgacttggccaccttcgggttccccatctcatgctggtacaaccccacctgaaaatggactctcagagcacccctgtgaaacagaacagataaatgcaaagagaaaagatacaaccagtgacaaagatgattcgctaggaagccaacaaacaaatgaacaatgtgctcaaaaggctgagccaacagagtcctgcgaacaaattgctgtccaagtgaataatggggatgctggaagggagatgccctgcccgttgccctgtgatgaagaaagcccagaggcagagctacacaaccatggaatccaaattaattcctgttctgtgcgactggtggatataaaaaaggaaaagccattttctaattcaaaagttgagtgccaagcccaagcaagaactcatcataaccaggcatctgacataatagtcatcagcagtgaggactctgaaggatccactgacgttgatgagcccttagaagtcttcatctcagcaccgagaagtgagcctgtgatcaataatgacaaccctttagaatcaaatgatgaaaaggagggccaagaagccacttgctcacgaccccagattgtaccagagcccatggatttcagaaaattatctacattcagagaaagttttaagaaaagagtgataggacaagaccacgacttttcagaatccagtgaggaggaggcgcccgcagaagcctcaagcggggcactgagaagcaagcatggtgagaaggctcctatgacttctagaagtacatctacttggagaatacccagcaggaagagacgtttcagcagtagtgacttttcagacctgagtaatggagaagagcttcaggaaacctgcagctcatccctaagaagagggtcaggatcacagccacaagaacctgaaaataagaagtgctcctgtgtcatgtgttttccaaaaggtgtgccaagaagccaagaagcaaggactgaaagtagtcaagcatctgacatgatggataccatggatgttgaaaacaattctactttggaaaaacacagtgggaaaagaagaaaaaagagaaggcatagatctaaagtaaatggtctccaaagagggagaaagaaagacagacctagaaaacatttaactctgaataacaaagtccaaaagaaaagatggcaacaaagaggaagaaaagccaacactagacctttgaaaagaagaagaaaaagaggtccaagaattcccaaagatgaaaatattaattttaaacaatctgaacttcctgtgacctgtggtgaggtgaagggcactctatataaggagcgattcaaacaaggaacctcaaagaagtgtatacagagtgaggataaaaagtggttcactcccagggaatttgaaattgaaggagaccgcggagcatccaagaactggaagctaagtatacgctgcggtggatataccctgaaagtcctgatggagaacaaatttctgccagaaccaccaagcacaagaaaaaagagaatactggaatctcacaacaataccttagttgacccttgtgaggagcataagaagaagaacccagatgcttcagtcaagttctcagagtttttaaagaagtgctcagagacatggaagaccatttttgctaaagagaaaggaaaatttgaagatatggcaaaggcggacaaggcccattatgaaagagaaatgaaaacctatatccctcctaaaggggagaaaaaaaagaagttcaaggatcccaatgcacccaagaggcctcctttggcctttttcctgttctgctctgagtatcgcccaaaaatcaaaggagaacatcctggcctgtccattgatgatgttgtgaagaaactggcagggatgtggaataacaccgctgcagctgacaagcagttttatgaaaagaaggctgcaaagctgaaggaaaaatacaaaaaggatattgctgcatatcgagctaaaggaaagcctaattcagcaaaaaagagagttgtcaaggctgaaaaaagcaagaaaaagaaggaagaggaagaagatgaagaggatgaacaagaggaggaaaatgaagaagatgatgataaataagttgcttctagtgcagtttttttcttgtctataaagcatttaagctgcctgtacacaactcactccttttaaagaaaaaaacttcaacgtaagactgtgtaagatttgtttttaaaccgtacactgtgtttttttgtatagttaaccactaccgaatgtgtcttcagatagccctgtcctggtggtatttagccactaacctttgcctggtacagtatgggggttgtaaattggcatggaaatttaaagcaggttcttgttagtgcacagcacaaattagttgtataggaggatggtagttttttcaccttcagttgtctctgatgtagcttatacaaaacatttgttgttctgttaactgaatgccactctgtaattgcaaaaaaaaaaaacagttgcagctgttttgttgacattctgaatgcttctaagtaaatacaatttttaaaaaaccgtatgagggaactgtgtagacaaggtaccaggtcagtcttcttccatgttctattagctccacaaagccaatctcaatccctcaaaacaatcttgtcatacttgaaaatatgacactctagtcaaagccttggtaaaataatcagtgtttccaatctgtcctgttacaaaagaaacagattattattgaacttatgcaaataaccattgtcataagaatgtttatgaatagtttccaaattatggcaaattcatgtagagagagaaaagtaactgttttggttttgctcacaaaagtctactttacctaagggctgtcagatataagtaacttaaaagaaagagaagttttcttgacttttgaaaacaaaatatgaaaagaatcggcaatgtttcaaacaaaaagtcataaaagtcactttattcctccatcaaaaaaaaaaaaaaaaaaaaaa

SEQ ID NO: 3 - DNA, 398 bp, Homo sapiens
agttcaaaggcagataaatctgtaaattattttatcctatctaccatttcttaagaagacattactccaaaataattaaatttaaggctttatcaggtctgcatatagaatcttaaattctaataaagtttcatgttaatgtcataggatttttaaaagagctataggtaatttctgtataatatgtgtatattaaaatgtaattgatttcagttgaaagtattttaaagctgataaatagcattagggttctttgcaatgtggtatctagctgtattattggttttatttactttaaacattttgaaaagcttatactggcagcctagaaaaacaaacaattaatgtatctttatgtccctggcacatgaataaactttgctgtggtttactaatct

SEQ ID NO: 4 - DNA, 2787 bp, Homo sapiens
agagcggaggccgcactccagcactgcgcagggaccgccttggaccgcagttgccggccaggaatcccagtgtcacggtggacacgcctccctcgcgcccttgccgcccacctgctcacccagctcaggggctttggaattctgtggccacactgcgaggagatcggttctgggtcggaggctacaggaagactcccactccctgaaatctggagtgaagaacgccgccatccagccaccattccaaggaggtgcaggagaacagctctgtgataccatttaacttgttgacattacttttatttgaaggaacgtatattagagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacatcgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaaattaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcag

SEQ ID NO: 5 - DNA, 2808 bp, Homo sapiens
gcggcggcggcggcgcagtttgctcatactttgtgacttgcggtcacagtggcattcagctccacacttggtagaaccacaggcacgacaagcatagaaacatcctaaacaatcttcatcgaggcatcgaggtccatcccaataaaaatcaggagaccctggctatcatagaccttagtcttcgctggtatactcgctgtctgtcaaccagcggttgactttttttaagccttcttttttctcttttaccagtttctggagcaaattcagtttgccttcctggatttgtaaattgtaatgacctcaaaactttagcagttcttccatctgactcaggtttgcttctctggcggtcttcagaatcaacatccacacttccgtgattatctgcgtgcattttggacaaagcttccaaccaggatacgggaagaagaaatggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttagagcccttctgaaaaagtatcctgcttctgatatgcagttttccttaaattatctaaaatctgctagggaatatcaatagatatttaccttttattttaatgtttcctttaattttttactatttttactaatctttctgcagaaacagaaaggttttcttctttttgcttcaaaaacattcttacattttactttttcctggctcatctctttattcttttttttttttttaaagacagagtctcgctctgttgcccaggctggagtgcaatgacacagtcttggctcactgcaacttctgcctcttgggttcaagtgattctcctgcctcagcctcctgagtagctggattacaggcatgtgccacccacccaactaatttttgtgtttttaataaagacagggtttcaccatgttggccaggctggtctcaaactcctgacctcaagtaatccacctgcctcggcctcccaaagtgctgggattacagggatgagccaccgcgcccagcctcatctctttgttctaaagatggaaaaaccacccccaaattttctttttatactattaatgaatcaatcaattcatatctatttattaaatttctaccgcttttaggccaaaaaaatgtaagatcgttctctgcctcacatagcttacaagccagctggagaaatatggtactcattaaaaaaaaaaaaaaagtgatgtacaacc

SEQ ID NO: 6 - DNA, 1260 bp, Homo sapiens
gggggtggggtccccggggcggggcggggcgcgctgtgtcgcgggtcggagctcggtcctgctggaggccacgggtgccacacactcggtcccgacatgatggcgagcatgcgagtggtgaaggagctggaggatcttcagaagaagcctcccccatacctgcggaacctgtccagcgatgatgccaatgtcctggtgtggcacgctctcctcctacccgaccaacctccctaccacctgaaagccttcaacctgcgcatcagcttcccgccggagtatccgttcaagcctcccatgatcaaattcacaaccaagatctaccaccccaacgtggacgagaacggacagatttgcctgcccatcatcagcagtgagaactggaagccttgcaccaagacttgccaagtcctggaggccctcaatgtgctggtgaatagaccgaatatcagggagcccctgcggatggacctcgctgacctgctgacacagaatccggagctgttcagaaagaatgccgaagagttcaccctccgattcggagtggaccggccctcctaactcatgttctgaccctctgtgcactggatcctcggcatagcggacggacacacctcatggactgaggccagagccccctgtggcccattccccattcatttttcccttcttaggttgttagtcattagtttgtgtgtgtgtgtggtggagggaagggagctatgagtgtgtgtgttgtgtatggactcactcccaggttcacctggccacaggtgcacccttcccacaccctttacattccccagagccaagggagtttaagtttgcagttacaggccagttctccagctctccatcttagagagacaggtcaccttgcaggcctgcttgcaggaaatgaatccagcagccaactcgaatccccctagggctcaggcactgagggcctggggacagtggagcatatgggtgggagacagatggagggtaccctatttacaactgagtcagccaagccactgatgggaatatacagatttaggtgctaaaccgtttattttccacggatgagtcacaatctgaagaatcaaacttccatcctgaaaatctatatgtttcaaaaccacttgccatcctgttagattgccagttcctgggaccaggcctcagactgtgaagtatatatcctccagcattcagtccagggggagccacggaaaccatgttcttgcttaagccattaaagtcagagatgaattctgg

SEQ ID NO: 7 - DNA, 983 bp, Homo sapiens
gtggaattcatggcatctacttcgtatgactattgcagagtgcccatggaagacggggataagcgctgtaagcttctgctggggataggaattctggtgctcctgatcatcgtgattctgggggtgcccttgattatcttcaccatcaaggccaacagcgaggcctgccgggacggccttcgggcagtgatggagtgtcgcaatgtcacccatctcctgcaacaagagctgaccgaggcccagaagggctttcaggatgtggaggcccaggccgccacctgcaaccacactgtgatggccctaatggcttccctggatgcagagaaggcccaaggacaaaagaaagtggaggagcttgagggagagatcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtgagatcccaggaagctggcacatcttggaaggtccgtcctgctcggcttttcgcttgaacattcccttgatctcatcagttctgagcgggtcatggggcaacacggttagcggggagagcacggggtagccggagaagggcctctggagcaggtctggaggggccatggggcagtcctgggtgtggggacacagtcgggttgacccagggctgtctccctccagagcctccctccggacaatgagtcccccctcttgtctcccaccctgagattgggcatggggtgcggtgtggggggcatgtgctgcctgttgttatgggttttttttgcggggggggttgcttttttctggggtctttgagctccaaaaaataaacacttcctttgagggagagcaaaaaaaaaaaaaaaaaaaaaaaaaaaaaaaaaaaa

SEQ ID NO: 8 - DNA, 634 bp, Homo sapiens
cggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagattggcgtgcacgccttccagcagcgtctggctgtccacccgagcggtgtggcgctgcaggacagggtcccccttgccagccagggcctgggccctggcagcacggtcctgctggtggtggacaaatgcgacgaacctctgagcatcctggtgaggaataacaagggccgcagcagcacctacgaggtccggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaagagaat

SEQ ID NO: 9 - DNA, 768 bp, Homo sapiens
ccttcagcataaaagctgatccacaaacaagaggagcaccagacctcctcttggcttcgagatggcttcgccacaccaagagcccaaacctggagacctgattgagattttccgccttggctatgagcactgggccctgtatataggagatggctacgtgatccatctggctcctccaagtgagtaccccggggctggctcctccagtgtcttctcagtcctgagcaacagtgcagaggtgaaacgggggcgcctggaagatgtggtgggaggctgttgctatcgggtcaacaacagcttggaccatgagtaccaaccacggcccgtggaggtgatcatcagttctgcgaaggagatggttggtcagaagatgaagtacagtattgtgagcaggaactgtgagcactttgtcgcccagctgagatatggcaagtcccgctgtaaacaggtggaaaaggccaaggttgaagtcggtgtggccacggcgcttggaatcctggttgttgctggatgctcttttgcgattaggagataccaaaaaaaagcaacagcctgaagcagccacaaaatcctgtgttagaagcagctgtgggggtcccagtggagatgagcctcccccatgcctccagcagcctgaccctcgtgccctgtctcaggcgttctctagatcctttcctctgtttccctctctcgctggcaaaagtatgatctaattgaaacaagactgaaggatcaataaacagccatctgccccttcaaaaaaaaaaa

SEQ ID NO: 10 - DNA, 337 bp, Homo sapiens
gcctcctgcagcccccatagcagattctgagaacaataactccacaatggcgtcggcctcggagggtgaaatggagtgtgggcaggagctgaaggaggaagggggcccgtgcttgttcccgggctcagacagttggcaagaaaaccccgaggagccctgttccaaagcctcctggaccgtccaagaaggagctacatcagaggttttggtagatgctgctgtagacctcatatccgatgaatgggaagctgctaatgccatacccagcaagagaaggaagcaggatgcagccccgcttgaggccgccagcgtgccttctgcagactgtgagcagagc

SEQ ID NO: 11 - DNA, 2254 bp, Homo sapiens
aatcgaaagtagactcttttctgaagcatttcctgggatcagcctgaccacgctccatactgggagaggcttctgggtcaaaggaccagtctgcagagggatcctgtggctggaagcgaggaggctccacacggccgttgcagctaccgcagccaggatctgggcatccaggcacggccatgacccctccgaggctcttctgggtgtggctgctggttgcaggaacccaaggcgtgaacgatggtgacatgcggctggccgatgggggcgccaccaaccagggccgcgtggagatcttctacagaggccagtggggcactgtgtgtgacaacctgtgggacctgactgatgccagcgtcgtctgccgggccctgggcttcgagaacgccacccaggctctgggcagagctgccttcgggcaaggatcaggccccatcatgctggacgaggtccagtgcacgggaaccgaggcctcactggccgactgcaagtccctgggctggctgaagagcaactgcaggcacgagagagacgctggtgtggtctgcaccaatgaaaccaggagcacccacaccctggacctctccagggagctctcggaggcccttggccagatctttgacagccagcggggctgcgacctgtccatcagcgtgaatgtgcagggcgaggacgccctgggcttctgtggccacacggtcatcctgactgccaacctggaggcccaggccctgtggaaggagccgggcagcaatgtcaccatgagtgtggatgctgagtgtgtgcccatggtcagggaccttctcaggtacttctactcccgaaggattgacatcaccctgtcgtcagtcaagtgcttccacaagctggcctctgcctatggggccaggcagctgcagggctactgcgcaagcctctttgccatcctcctcccccaggacccctcgttccagatgcccctggacctgtatgcctatgcagtggccacaggggacgccctgctggagaagctctgcctacagttcctggcctggaacttcgaggccttgacgcaggccgaggcctggcccagtgtccccacagacctgctccaactgctgctgcccaggagcgacctggcggtgcccagcgagctggccctactgaaggccgtggacacctggagctggggggagcgtgcctcccatgaggaggtggagggcttggtggagaagatccgcttccccatgatgctccctgaggagctctttgagctgcagttcaacctgtccctgtactggagccacgaggccctgttccagaagaagactctgcaggccctggaattccacactgtgcccttccagttgctggcccggtacaaaggcctgaacctcaccgaggatacctacaagccccggatttacacctcgcccacctggagtgcctttgtgacagacagttcctggagtgcacggaagtcacaactggtctatcagtccagacgggggcctttggtcaaatattcttctgattacttccaagccccctctgactacagatactacccctaccagtccttccagactccacaacaccccagcttcctcttccaggacaagagggtgtcctggtccctggtctacctccccaccatccagagctgctggaactacggcttctcctgctcctcggacgagctccctgtcctgggcctcaccaagtctggcggctcagatcgcaccattgcctacgaaaacaaagccctgatgctctgcgaagggctcttcgtggcagacgtcaccgatttcgagggctggaaggctgcgattcccagtgccctggacaccaacagctcgaagagcacctcctccttcccctgcccggcagggcacttcaacggcttccgcacggtcatccgccccttctacctgaccaactcctcaggtgtggactagacgcgtggccaagggtggtgagaaccggagaaccccaggacgccctcactgcaggctcccctcctcggcttccttcctctctgcaatgaccttcaacaaccggccaccagatgtcgccctactcacctgaggctcagcttcaagaaattactggaaggcttccactagggtccaccaggagttctcccaccacctcaccagtttccaggtggtaagcaccaggaggccctcgaggttgctctggatccccccacagcccctggtcagtctgcccttgtcactggtctgaggtcattaaaattacattgaggttccta

SEQ ID NO: 12 - DNA, 2815 bp, Homo sapiens
aggaagcggaggaaggtgaagtaggaccgaattcctgtgccgaagaggcctgcagtgggagagcaggatgggggctccggaggtggcgcccaggctctgagctaccctaggtctgcagactagcgggcattggccagagacatggcccagccactggccttcatcctcgatgtccctgagaccccaggggaccagggccagggccccagcccctatgatgaaagcgaagtgcacgactccttccagcagctcatccaggagcagagccagtgcacggcccaggaggggctggagctgcagcagagagagcgggaggtgacaggaagtagccagcagacactctggcggcccgagggcacccagagcacggccacactccgcatcctggccagcatgcccagccgcaccattggccgcagccgaggtgccatcatctcccagtactacaaccgcacggtgcagcttcggtgcaggagcagccggcccctgctcgggaactttgtccgctccgcctggcccagcctccgcctgtacgacctggagctggaccccacggccctggaggaggaggagaagcagagcctcctggtgaaggagttccagagcctggcagtggcacagcgggaccacatgcttcgcgggatgcccttaagcctggctgagaaacgcagcctgcgagagaagagcaggaccccgagggggaagtggaggggccagccgggcagcggcggggtctgctcctgctgtggccggctcagatatgcctgcgtgctggccttgcacagcctgggcctggcgctgctctccgccctgcaggccctgatgccgtggcgctacgccctgaagcgcatcgggggccagttcggctccagcgtgctctcctacttcctctttctcaagaccctgctggctttcaatgccctcctgctgctgctgctggtggccttcatcatgggccctcaggtcgccttcccacccgccctgccgggccctgcccccgtctgcacaggcctggagctcctcacaggcgcgggttgcttcacccacaccgtcatgtactacggccactacagtaacgccacgctgaaccagccgtgtggcagccccctggatggcagccagtgcacacccagggtgggtggcctgccctacaacatgcccctggcctacctctccactgtgggcgtgagcttctttatcacctgcatcaccctggtgtacagcatggctcactctttcggggagagctaccgggtgggcagcacctctggcatccacgccatcaccgtcttctgctcctgggactacaaggtgacgcagaagcgggcctcccgcctccagcaggacaatattcgcacccggctgaaggagctgctggccgagtggcagctgcggcacagccccaggagcgtgtgcgggaggctgcggcaggcggctgtgctggggcttgtgtggctgctgtgtctggggaccgcgctgggctgcgccgtggccgtccacgtcttctcggagttcatgatccagagtccagaggctgctggccaggaggctgtgctgctggtcctgcccctggtggttggcctcctcaacctgggggccccctacctgtgccgtgtcctggccgccctggagccgcatgactccccggtactggaggtgtacgtggccatctgcaggaacctcatcctcaagctggccatcctggggacactgtgctaccactggctgggccgcagggtgggcgtcctgcagggccagtgctgggaggattttgtgggccaggagctgtaccggttcctggtgatggacttcgtcctcatgttgctggacacgctttttggggaactggtgtggaggattatctccgagaagaagctgaagaggaggcggaagccggagtttgacattgcccggaatgtcctggagctgatttatgggcagactctgacctggctgggggtgctcttctcgcccctcctccccgccgtgcagatcatcaagctgctgctcgtcttctatgtcaagaagaccagccttctggccaactgccaggcgccgcgccggccctggctggcctcacacatgagcaccgtcttcctcacgctgctctgcttccccgccttcctgggcgccgctgtcttcctctgctacgccgtctggcaggtgaagccctcgagcacctgcggccccttccggaccctggacaccatgtacgaggccggcagggtgtgggtgcgccacctggaggcggcaggccccagggtctcctggctgccctgggtgcaccggtacctgatggaaaacaccttctttgtcttcctggtgtcagccctgctgctggccgtgatctacctcaacatccaggtggtgcggggccagcgcaaggtcatctgcctgctcaaggagcagatcagcaatgagggtgaggacaaaatcttcttaatcaacaagcttcactccatctacgagaggaaggagagggaggagaggagcagggttgggacaaccgaggaggctgcggcaccccctgccctgctcacagatgaacaggatgcctagggggacggcgatgggcctcacgggcccgcccagcaccctgagaccacactgttgcctcccagtgaccctgctgggacaccaggacaaggaagacagtttcgcctctcgaaagccgcagctgcgcctaggctggagctggaagggtgggtgaatccggcttgggcatccccaatgaactctgccctgcctgggactctatttattctgattaaaggggttttgcaaatgggaaaaaaaaaaaaaaaaaaaaaaaaaa

SEQ ID NO: 13 - DNA, 7204 bp, Homo sapiens
gccccagcactcgccggcggcagtgaaaggacgcgccggagccggataacagaaagtaacgtgaaggaattcaggtgactcagacatggaggagagaagacctcatctggatgccaggcccaggaattcccataccaaccacagaggccctgtggatggagagttaccaccaagagctagaaatcaggccaataacccaccagccaatgctctccgaggaggagccagccaccctggaaggcatcctagggccaacaaccatcctgctgcttactggcagagggaagagagatttagggccatgggcaggaacccacatcaaggaaggaggaaccaggaggggcatgccagcgacgaagctagagaccaaagacatgaccaggagaatgacaccaggtggagaaatggcaaccaggactgtaggaaccgcagaccaccatggtccaatgacaacttccagcagtggcggactccccaccagaagcctacagaacagccacagcaggcgaagaaactgggctacaagttcttagaaagtcttctgcagaaagacccttctgaggtggtcatcacacttgccacaagtttagggctgaaagagctcctttctcattcttccatgaaatctaacttccttgagctcatctgtcaggttcttcggaaggcttgtagctccaaaatggatcgccagagtgttctccatgtactgggcatattgaaaaactccaaatttctcaaagtctgcctgcctgcttatgtggtagggatgatcactgaacccatccctgacatccgaaaccagtatccagagcacataagcaacatcatctccctcctccaggaccttgtaagtgtcttccctgccagctctgtgcaggaaacttccatgctggtttccctcctgccaacctctcttaatgctctgagagcctctggtgttgacatagaagaggaaacggagaagaacctggaaaaggtacagactatcattgaacatctgcaggaaaagaggcgagagggcactttgagagtggatacctacactctagtgcagcctgaggcagaagaccatgttgagagctaccgaaccatgcccatttaccctacctacaatgaagtgcacttggatgagaggcccttccttcgccccaatatcatttctggaaaatacgacagcactgctatctatctggatacccacttccggctcctgcgagaagatttcgtcagacctttacgggaaggtattttggaacttctccaaagctttgaagaccagggcctgaggaagagaaagtttgatgacatccgaatctactttgacaccaggattatcacccccatgtgttcatcatcaggcatagtctacaaggtgcagtttgacacaaaaccactgaagtttgttcgctggcagaattccaaacgattgctctatgggtctttggtatgcatgtccaaggacaacttcgagacatttctttttgccaccgtatctaacagggagcaggaagatctctgccgaggaattgtccagctctgcttcaatgagcaaagccaacagctgctagcagaggtccagccctctgactctttcctcatggtagagacaactgcatactttgaggcctacaggcacgtcctggaaggactccaggaggtccaggaggaagatgttcccttccagaggaatatcgtggagtgtaactctcatgtgaaggagccaaggtacttgctaatggggggcagatatgactttacccccttaatagagaatccttcagccactggggaatttctaagaaatgtcgagggtttgagacatcccagaattaatgtcttagatcctggccagtggccctcaaaagaagccctgaagctggatgactcccagatggaagccttgcagtttgctctcacaagggaactggctattattcaaggacctcctggaacaggcaaaacctatgtgggtctaaaaattgttcaggccctcctaaccaacgagtctgtttggcaaattagcctccagaagttccccatcttggttgtgtgttatactaatcatgctttggaccagtttctggaaggcatctacaattgtcagaagaccagcattgtgcgggtgggtggaaggagcaacagtgaaatcctgaagcagttcaccctaagggagctgaggaacaagcgggaattccgccgcaacctccccatgcacctccgaagggcctacatgagtatcatgacacagatgaaggagtcagagcaagagcttcatgaaggagccaagaccctggagtgcaccatgcgtggtgtcctacgggaacagtacctgcagaagtacatctcaccccagcactgggaaagtctcatgaatggaccagtgcaggatagtgaatggatttgcttccagcactggaagcattccatgatgctggagtggctaggtcttggtgtcggttctttcacgcaaagtgtttctccagcaggacctgagaatacagcccaggcagaaggggatgaggaggaagaaggggaggaggagagttcgctgatagagatcgcagaggaagctgacctgattcaagcagaccgggtgattgaggaggaagaggtggtgaggccccagcggcggaagaaggaagagagtggagcagaccaggagttggctaaaatgcttctggccatgaggctagaccattgtggcactgggacagcagctggacaggagcaagccacaggagagtggcagacccagcgcaaccagaaaaagaaaatgaaaaaaagagtgaaggatgagcttcgcaaactgaacaccatgactgcagccgaggccaacgagatcgaggatgtttggcacctggacctcagttctcgctggcagctttataggctctggctacagttgtaccaggctgacacccgccggaagatcctcagctatgaacgccagtaccgcacatcagcagaaagaatggccgagctgagactccaggaagacctgcacattcttaaagatgcccaggttgtaggaatgacaaccacaggtgctgccaaataccgccagatcctacagaaggtggagccgaggattgtcatagtggaagaagctgcggaagtccttgaggcccataccattgccacattgagcaaagcttgccagcacctcattttgattggggaccaccagcagctgcgccccagtgccaacgtgtatgatctggccaagaacttcaaccttgaggtgtccctttttgaacggctagtgaaagtaaacattccctttgtccgtctgaattaccagcaccgtatgtgccctgaaattgcccgccttttgaccccccacatttaccaggatctggagaatcatccatctgttcttaagtatgagaagattaagggggtgtcttccaaccttttctttgtagaacacaactttcctgaacaggaaatccaagagggcaaaagccatcagaaccagcatgaggctcactttgtggtagagctgtgcaagtacttcctgtgccaggaatacctgccttcccagatcaccatcctcactacctataccgggcagctcttctgcctgcgcaaactgatgcctgccaagacatttgctggcgtcagggtccatgttgtggacaaataccaaggggaagagaatgacatcatcctcctctcgctagtgcggagcaaccaagaaggcaaggtgggttttctgcagatatccaaccgcatctgtgtggccttgtcccgagccaagaagggaatgtactgcatcggaaacatgcagatgctggccaaggtgcccctgtggagcaagatcattcatacacttcgagagaacaatcaaataggccccatgctccggctctgctgccagaaccaccctgaaacccacaccttagtatccaaagcttctgacttccaaaaagtacccgaaggaggctgcagcctgccctgcgagttccgcctgggctgtgggcatgtctgcacccgtgcctgccacccttatgactcttcacacaaggagttccaatgcatgaagccatgccagaaggtcatctgtcaggaagggcaccggtgtccccttgtttgcttccaggagtgtcagccttgtcaggtgaaggtgcccaaaatcattcctcggtgcggccatgaacaaatggtcccttgttccgtgcctgagtcagatttctgctgccaggagccttgctccaagtctctgagatgtgggcacagatgcagccacccatgtggtgaggactgtgtgcagttgtgttcagaaatggtcaccataaaactcaagtgtgggcacagtcaaccggtaaaatgtggtcatgtggaaggcctcctgtatggtggtctgctagtcaagtgtaccacaaagtgtggcactatcttggactgcgggcatccttgcccaggctcctgccacagctgcttcgaagggcgtttccatgaacgctgtcagcagccctgcaagcgcctgcttatctgctcacacaagtgccaggaaccatgcattggtgagtgcccaccctgccagcggacctgtcagaaccgctgtgtccacagccagtgcaagaagaaatgtggggagctgtgtagtccctgcgtggaaccctgtgtctggcgctgccagcactaccagtgcaccaaactctgctctgagccctgcaaccgacccccatgctatgtgccttgtactaagctgctagtttgtggccacccctgcattggtctctgtggggagccatgccccaagaaatgccggatctgccacatggatgaggtcacccaaatattctttggctttgaggatgagcctgatgcccgctttgtgcagctggaagactgcagccacatctttgaggtgcaagccctagaccgctacatgaatgaacagaaggatgatgaagtcgccatcagattgaaagtctgccctatctgccaggtgcccatccgcaaaaacctgaggtatggaactagcataaaacagcggctagaagagattgaaatcatcaaggaaaagatccagggctcagcaggggaaatagcaaccagccaggaacggcttaaggccctgctggagaggaagagcctcctccaccagctgcttcctgaagacttcctgatgttaaaggagaagctggcccagaaaaatctgtcagtgaaggacctgggtctggttgagaattacatcagcttctatgaccacctggccagcctgtgggattccctgaaaaagatgcatgtcttagaagagaaaagagtgaggactcgactagaacaggtccatgagtggctggccaagaagcgcttgagcttcactagccaggaactaagtgacctccgaagtgaaatccagaggctcacatacctggtgaaccttctgacccgctacaagatagcagagaagaaggtgaaagatagcatagcagtagaggtctatagtgtccagaatatccttgagaaaacatgtaagttcacccaagaggatgaacaacttgtgcaggaaaagatggaagctctgaaagccacccttccctgctctggcctgggcatctcagaggaagagcgagtgcagattgtcagtgccataggttatcctcgtggtcactggttcaagtgccgcaatggccatatctatgtgattggcgattgtgggggagccatggagaggggcacgtgtcctgactgtaaggaagtgattggtggcacaaatcatactctggaaagaagcaaccagcttgcttctgaaatggatggagcccagcatgctgcctggtctgacacggccaacaacctgatgaactttgaggagatccaggggatgatgtaggaagatggtacaccactgccttttgccctcgccactgaatgactggggccagctccctaatgaaggaactgaagtttgttttttattatcatcctttttaggctgggcgcagtggcttacgcctgtaatcccagcactttgggaggccgaggcaggcggatcacgaggtcaggagttcgagaccagcctgaccaacatggcgaaaccccgtctctactaaaaatacaaaaattagctgggcgttatggcgggcgcctgtaatcccagctacttgggaggctgaggcagaagaatcgcttaaacccaggaggcggaggttgcagtgagctgagatcatgccattgcactccagtctgggcgacaggagcaagactctgtctcaaaaaaaaaaaatcattctttttagtcttagcacctacttaaggatccacttttagggctcacccacatttgtttctagatttacccctgcgctagagtaagcactttatctccagaactgagagcaaagttaacaaatctcaccccttctctcctgcaaattagtggacagactccctggaacatgtttggggcttccacctagggccacctagtggtatctctgggtctttacttggtcagatgtttattctacattgttccccaggaacagagtatgagctcattgatgcagaccgattctaattgccaggccctaatttgcagactaactctcataataaacagaggcccatagttgtttatgaactgcttatcccttaaaggagcacaagaacccctccctgccctccttgggcaccctgcctccaggagatggaggcacgtgataagacaaaagactgcaccaactcaccctgacacagttacatagtcactgagagtggggaagatgggacagcccacatgctgcataagatgggccttatgcagcaggcccaggtcgtcattaaggagtgacccctttcctgtaacctgcactttgggatggtagaagtttctttacctgctgacaggtttggtggcactgctggttacccctgggccctgaatggagctaaaatcacatttggtaccagcagcacctatcccaagtgtgatccttcatcccaacactccctcttggagctgttccctgggtagagctagcatgccagcagcttctgcaggctccaaacccaggccagaagccagacccaggcctgctgcctgcatctgcattccctccttccagtgttccttagaacagacatttaggtatctcaggtcctttctaagtgtccctttcctatgtatgcatttcctttttttgtctttactatgcactttagcttataaagccaattaaaaacgatgattgag

SEQ ID NO: 14 - DNA, 2054 bp, Homo sapiens
gggaagctcgggccggcagggtttccccgcacgctggcgcccagctcccggcgcggaggccgctgtaagtttcgctttccattcagtggaaaacgaaagctgggcggggtgccacgagcgcggggccagaccaaggcgggcccggagcggaacttcggtcccagctcggtccccggctcagtcccgacgtggaactcagcagcggaggctggacgcttgcatggcgcttgagagattccatcgtgcctggctcacataagcgcttcctggaagtgaagtcgtgctgtcctgaacgcgggccaggcagctgcggcctgggggttttggagtgatcacgaatgagcaaggcgtttgggctcctgaggcaaatctgtcagtccatcctggctgagtcctcgcagtccccggcagatcttgaagaaaagaaggaagaagacagcaacatgaagagagagcagcccagagagcgtcccagggcctgggactaccctcatggcctggttggtttacacaacattggacagacctgctgccttaactccttgattcaggtgttcgtaatgaatgtggacttcaccaggatattgaagaggatcacggtgcccaggggagctgacgagcagaggagaagcgtccctttccagatgcttctgctgctggagaagatgcaggacagccggcagaaagcagtgcggcccctggagctggcctactgcctgcagaagtgcaacgtgcccttgtttgtccaacatgatgctgcccaactgtacctcaaactctggaacctgattaaggaccagatcactgatgtgcacttggtggagagactgcaggccctgtatacgatccgggtgaaggactccttgatttgcgttgactgtgccatggagagtagcagaaacagcagcatgctcaccctcccactttctctttttgatgtggactcaaagcccctgaagacactggaggacgccctgcactgcttcttccagcccagggagttatcaagcaaaagcaagtgcttctgtgagaactgtgggaagaagacccgtgggaaacaggtcttgaagctgacccatttgccccagaccctgacaatccacctcatgcgattctccatcaggaattcacagacgagaaagatctgccactccctgtacttcccccagagcttggatttcagccagatccttccaatgaagcgagagtcttgtgatgctgaggagcagtctggagggcagtatgagctttttgctgtgattgcgcacgtgggaatggcagactccggtcattactgtgtctacatccggaatgctgtggatggaaaatggttctgcttcaatgactccaatatttgcttggtgtcctgggaagacatccagtgtacctacggaaatcctaactaccactggcaggaaactgcatatcttctggtttacatgaagatggagtgctaatggaaatgcccaaaaccttcagagattgacacgctgtcattttccatttccgttcctggatctacggagtcttctaagagattttgcaatgaggagaagcattgttttcaaactatataactgagccttatttataattagggatattatcaaaatatgtaaccatgaggcccctcaggtcctgatcagtcagaatggatgctttcaccagcagacccggccatgtggctgctcggtcctgggtgctcgctgctgtgcaagacattagccctttagttatgagcctgtgggaacttcaggggttcccagtggggagagcagtggcagtgggaggcatctgggggccaaaggtcagtggcagggggtatttcagtattatacaactgctgtgaccagacttgtatactggctgaatatcagtgctgtttgtaatttttcactttgagaaccaacattaattccatatgaatcaagtgttttgtaactgctattcatttattcagcaaatatttattgatcatctcttctccataagatagtgtgataaacacagtcatgaataaagttattttccacaaaaaaaaaaaaaaaaaaaaa

SEQ ID NO: 15 - DNA, 2961 bp, Homo sapiens
aagagatgatttctccatcctgaacgtgcagcgagcttgtcaggaagatcggaggtgccaagtagcagagaaagcatcccccagctctgacagggagacagcacatgtctaaggcccacaagccttggccctaccggaggagaagtcaattttcttctcgaaaatacctgaaaaaagaaatgaattccttccagcaacagccaccgccattcggcacagtgccaccacaaatgatgtttcctccaaactggcagggggcagagaaggacgctgctttcctcgccaaggacttcaactttctcactttgaacaatcagccaccaccaggaaacaggagccaaccaagggcaatggggcccgagaacaacctgtacagccagtacgagcagaaggtgcgcccctgcattgacctcatcgactccctgcgggctctgggtgtggagcaggacctggccctgccagccatcgccgtcatcggggaccagagctcgggcaagagctctgtgctggaggcactgtcaggagtcgcgcttcccagaggcagcggaatcgtaaccaggtgtccgctggtgctgaaactgaaaaagcagccctgtgaggcatgggccggaaggatcagctaccggaacaccgagctagagcttcaggaccctggccaggtggagaaagagatacacaaagcccagaacgtcatggccgggaatggccggggcatcagccatgagctcatcagcctggagatcacctcccctgaggttccagacctgaccatcattgaccttcccggcatcaccagggtggctgtggacaaccagccccgagacatcggactgcagatcaaggctctcatcaagaagtacatccagaggcagcagacgatcaacttggtggtggttccctgtaacgtggacattgccaccacggaggcgctgagcatggcccatgaggtggacccggaaggggacaggaccatcggtatcctgaccaaaccagatctaatggacaggggcactgagaaaagcgtcatgaatgtggtgcggaacctcacgtaccccctcaagaagggctacatgattgtgaagtgccggggccagcaggagatcacaaacaggctgagcttggcagaggcaaccaagaaagaaattacattctttcaaacacatccatatttcagagttctcctggaggaggggtcagccacggttccccgactggcagaaagacttaccactgaactcatcatgcatatccaaaaatcgctcccgttgttagaaggacaaataagggagagccaccagaaggcgaccgaggagctgcggcgttgcggggctgacatccccagccaggaggccgacaagatgttctttctaattgagaaaatcaagatgtttaatcaggacatcgaaaagttagtagaaggagaagaagttgtaagggagaatgagacccgtttatacaacaaaatcagagaggattttaaaaactgggtaggcatacttgcaactaatacccaaaaagttaaaaatattatccacgaagaagttgaaaaatatgaaaagcagtatcgaggcaaggagcttctgggatttgtcaactacaagacatttgagatcatcgtgcatcagtacatccagcagctggtggagcccgcccttagcatgctccagaaagccatggaaattatccagcaagctttcattaacgtggccaaaaaacattttggcgaatttttcaaccttaaccaaactgttcagagcacgattgaagacataaaagtgaaacacacagcaaaggcagaaaacatgatccaacttcagttcagaatggagcagatggttttttgtcaagatcagatttacagtgttgttctgaagaaagtccgagaagagatttttaaccctctggggacgccttcacagaatatgaagttgaactctcattttcccagtaatgagtcttcggtttcctcctttactgaaataggcatccacctgaatgcctacttcttggaaaccagcaaacgtctcgccaaccagatcccatttataattcagtattttatgctccgagagaatggtgactccttgcagaaagccatgatgcagatactacaggaaaaaaatcgctattcctggctgcttcaagagcagagtgagaccgctaccaagagaagaatccttaaggagagaatttaccggctcactcaggcgcgacacgcactctgtcaattctccagcaaagagatccactgaagggcggcgatgcctgtggttgttttcttgtgcgtactcattcattctaaggggagtcggtgcaggatgccgcttctgctttggggccaaactcttctgtcactatcagtgtccatctctactgtactccctcagcatcagagcatgcatcaggggtccacacaggctcagctctctccaccacccagctcttccctgaccttcacgaagggatggctctccagtccttgggtcccgtagcacacagttacagtgtcctaagatactgctatcattcttcgctaatttgtatttgtattcccttccccctacaagattatgagaccccagagggggaaggtctgggtcaaattcttcttttgtatgtccagtctcctgcacagcacctgcagcattgtaactgcttaataaatgacatctcactgaacgaatgagtgctgtgtaagtgatggagatacctgaggctattgctcaagcccaggccttggacatttagtgactgttagccggtccctttcagatccagtggccatgccccctgcttcccatggttcactgtcattgtgtttcccagcctctccactcccccgccagaaaggagcctgagtgattctcttttcttcttgtttccctgattatgatgagcttccattgttctgttaagtcttgaagaggaatttaataaagcaaagaaactttttaaaaacgt

SEQ ID NO: 16 - DNA, 3539 bp, Homo sapiens
caagagttggtaagctcgctgcagtgggtggagagaggcctctagacttcagtttcagtttcctggctctgggcagcagcaagaattcctctgcctcccatcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaaccgtactggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgttctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctctgcatcttctggaaggtcaattacaactttgaagatgagaccgtgaggaagtttctactgagccagttgcagaaaaccaggcctgtgatcttggacccagccgaacccacaggtgacgtgggtggaggggaccgttggtgttggcatcttctggcaaaagaagcaaaggaatggttatcctctccctgcttcaaggatgggactggaaacccaataccaccttggaaagtgccgacaatgcagacaccaggaagttgtggagctaggatccatcctattgtcaatgagatgttctcatccagaagccatagaatcctgaataataattctaaaagaaacttctagagatcatctggcaatcgcttttaaagactcggctcaccgtgagaaagagtcactcacatccattcttcccttgatggtccctattcctccttcccttgcttcttggacttcttgaaatcaatcaagactgcaaaccctttcataaagtcttgccttgctgaactccctctctgcaggcagcctgcctttaaaaatagttgctgtcatccactttatgtgcatcttatttctgtcaacttgtattttttttcttgtatttttccaattagctcctcctttttccttccagtctaaaaaaggaatcctctgtgtcttcaaagcaaagctctttactttccccttggttctcataactctgtgatcttgctctcggtgcttccaactcatccacgtcctgtctgtttcctctgtatacaaaaccctttctgcccctgctgacacagacatcctctatgccagcagccagccaaccctttcattagaacttcaagctctccaaaggctcagattataactgttgtcatatttatatgaggctgttgtcttttccttctgagcctgcctttctcccccccacccaggagtatcctcttgccaaatcaaaagactttttccttgggctttagccttaaagatacttgaaggtctaggtgctttaacctcacataccctcacttaaacttttatcactgttgcatataccagttgtgatacaataaagaatgtatctggattttgtgcctagttcctagcacacagcttcaaaaattctagagtttcctgataggagtgtcttttgtattcataacaagcccttttcacccatgcctgggtttatgctaacaaggttacccatggtgggcccttagtttcaaggaaggagttggccaagccagaaagaccaagcatgtggttaaagcattggaattttcagccccatcccacccccaatctccaaggaggtgatggggctggaaattgagttcaattttaacatggccagtgatttaagcaatgctgcctatgtaaagaaaccccaataaaaactctggacagtgaggcttggggagcttcctgattggcagacattccaatgtactaggaaggtagcgcatcttgattccacagggacaaaggctcctgagctctgggcccttccagtgcttgccaccctacatactctttgtctggctcttcatttgtattctttataataaaatggtgattgtaagtagagca

SEQ ID NO: 17 - DNA, 1815 bp, Homo sapiens
ggggagatgatccgagccgcgccgccgccgctgttcctgctgctgctgctgctgctgctgctagtgtcctgggcgtcccgaggcgaggcagcccccgaccaggacgagatccagcgcctccccgggctggccaagcagccgtctttccgccagtactccggctacctcaaaagctccggctccaagcacctccactactggtttgtggagtcccagaaggatcccgagaacagccctgtggtgctttggctcaatgggggtcccggctgcagctcactagatgggctcctcacagagcatggccccttcctggtccagccagatggtgtcaccctggagtacaacccctattcttggaatctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactgatgaccacagcaaccagctccacggcctgatgcagcccctcccagcctctcccgctaggagagtcctcttctaagcaaagtgcccctgcaggcgggttctgccgccaggactgcccccttcccagagccctgtacatcccagactgggcccagggtctcccatagacagcctgggggcaagttagcactttattcccgcagcagttcctgaatggggtggcctggccccttctctgcttaaagaatgccctttatgatgcactgattccatcccaggaacccaacagagctcaggacagcccacagggaggtggtggacggactgtaattgatagattgattatggaattaaattgggtacagcttc

SEQ ID NO: 18 - DNA, 836 bp, Homo sapiens
ccagccttcagccggagaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaacactt

SEQ ID NO: 19 - DNA, 2077 bp, Homo sapiens
ccgagcgccagcgcggggaaccgggaaaaggaaaccgtgttgtgtacgtaagattcaggaaacgaaaccaggagccgcgggtgttggcgcaaaggttactcccagacccttttccggctgacttctgagaaggttgcgcacagctgtgcccggcagtctagaggcgcagaagaggaagccatcgcctggccccggctctctggaccttgtctcgctcgggagcggaaacagcggcagccagagaactgttttaatcatggacaaacaaaactcacagatgaatgcttctcacccggaaacaaacttgccagttgggtatcctcctcagtatccaccgacagcattccaaggacctccaggatatagtggctaccctgggccccaggtcagctacccacccccaccagccggccattcaggtcctggcccagctggctttcctgtcccaaatcagccagtgtataatcagccagtatataatcagccagttggagctgcaggggtaccatggatgccagcgccacagcctccattaaactgtccacctggattagaatatttaagtcagatagatcagatactgattcatcagcaaattgaacttctggaagttttaacaggttttgaaactaataacaaatatgaaattaagaacagctttggacagagggtttactttgcagcggaagatactgattgctgtacccgaaattgctgtgggccatctagaccttttaccttgaggattattgataatatgggtcaagaagtcataactctggagagaccactaagatgtagcagctgttgttgtccctgctgccttcaggagatagaaatccaagctcctcctggtgtaccaataggttatgttattcagacttggcacccatgtctaccaaagtttacaattcaaaatgagaaaagagaggatgtactaaaaataagtggtccatgtgttgtgtgcagctgttgtggagatgttgattttgagattaaatctcttgatgaacagtgtgtggttggcaaaatttccaagcactggactggaattttgagagaggcatttacagacgctgataactttggaatccagttccctttagaccttgatgttaaaatgaaagctgtaatgattggtgcctgtttcctcattgacttcatgttttttgaaagcactggcagccaggaacaaaaatcaggagtgtggtagtggattagtgaaagtctcctcaggaaatctgaagtctgtatattgattgagactatctaaactcatacctgtatgaattaagctgtaaggcctgtagctctggttgtatacttttgcttttcaaattatagtttatcttctgtataactgatttataaaggtttttgtacattttttaatactcattgtcaatttgagaaaaaggacatatgagtttttgcatttattaatgaaacttcctttgaaaaactgctttgaattatgatctctgattcattgtccattttactaccaaatattaactaaggccttattaatttttatataaattatatcttgtcctattaaatctagttacaatttatttcatgcataagagctaatgttattttgcaaatgccatatattcaaaaaagctcaaagataattttctttactattatgttcaaataatattcaatatgcatattatctttaaaaagttaaatgtttttttaatcttcaagaaatcatgctacacttaacttctcctagaagctaatctataccataatattttcatattcacaagatattaaattaccaattttcaaattattgttagtaaagaacaaaatgattctctcccaaagaaagacacattttaaatactccttcactctaaaactctggtattataacttttgaaagttaatatttctacatgaaatgtttagctcttacactctatccttcctagaaaatggtaattgagattactcagatattaattaaatacaatatcatatatatattcacagagtataaacctaaataatgatctattagattcaaatatttgaaataaaaacttgatttttttgt

SEQ ID NO: 20 - DNA, 1640 bp, Homo sapiens
aatgccacctgcttgaaggctatatgtgacaagtcactagaggttcacctgcaggttgacgccatgtacacaaatgtcaaagtaactaatatttgctctgatgggacactctactgccaggtgccttgtaagggtctgaacaagctcagtgaccttctacgtaagatagaggactacttccattgcaagcacatgacctctgagtgctttgtttcattacccttctgtgggaaaatctgcctcttccattgcaaaggaaaatggttacgagtagagatcacaaatgttcacagcagccgggctcttgatgttcagttcctggactctggcactgtgacatctgtaaaagtgtcagagctcagggaaattccacctcggtttctacaagaaatgattgcaataccacctcaggccattaagtgctgtttagcagatcttccacaatctattggcatgtggacaccagatgcagtgctgtggttaagagattctgttttgaattgctcggactgtagcattaaggttacaaaagtggatgaaaccagagggatcgcacatgtttatttatttacccctaagaacttccctgaccctcatcgcagtattaatcgccagattacaaatgcagacttgtggaagcatcagaaggatgtgtttttgagtgccatatccagtggagctgactctcccaacagcaaaaatggcaacatgcccatgtcgggcaacactggagagaatttcagaaagaacctcacagatgtcatcaaaaagtccatggtggaccatacgagcgctttctccacagaggaactgccacctcctgtccacttatcaaagccaggggaacacatggatgtgtatgtgcctgtggcctgtcacccaggctacttcgtcatccagccttggcaggagatacataagttggaagttctgatggaagagatgattctatattacagcgtgtctgaagagcgccacatagcagtggagaaagaccaagtgtatgctgcaaaagtggaaaataagtggcacagggtgcttttaaaaggaatcctgaccaatggactggtatctgtgtatgagctggattatggcaaacacgaattagtcaacataagaaaagtacagcccctagtggacatgttccgaaagctgcccttccaagcagtcacagctcaacttgcaggagtgaagtgcaaccagtggtctgaggaggcttctatggtgtttcgaaatcatgtggagaagaaacctctggtggcactggtgcagacagtcattgaaaatgctaacccttgggaccggaaagtagtggtctacttagtggacacatcgttgccagacaccgatacctggattcatgattttatgtcagagtatctgatagagctttcaaaagttaattaatgactgcctctgaaaccttgacaactaattcagattttttagcaataacaaaatgtagtaggcttaaaaaaaatcttaactctgctacatggctctgactgctgtgggggattgaaaagaatatgcttatgtttgatgaaagatatttaacaagttttgttttaacagagttgacttttcaaagaaaattgtacttgaattattactataatattagaataaaaatgtttatc

SEQ ID NO: 21 - DNA, 591 bp, Homo sapiens
agaaaatgcttctatttttctttagcacctccatggttctcatatacccatgtctgtaaaaagtgacatgagaattttgttgggttacattttattgtatttattagattcgcttatatagatgacttaggcagaaataaagtcatgtctttagaaggtgaacaagccaacttgtgatggcctgccttttgcttttggcagttgggatgagaacaattgactctcccattggttgttagatagttgaaatggtgcgttggtggtcatacttagtgttctaggctgtgaaatcatggagttcttccacttccaagaatgactcatttgctgttggattctagtacagaatttagcagcctgatgtgtccccaaactgatttaatttctactgaagtgcccttgtgtacatttgttttgtaatttaccaaagtactacctgagtgtataatgactcctgcagtgagttaatgtaattgctgctttgaccattgttttaaatctgtgtactagagtaactgtgagcagaatgaaatcacattatctcagtgttcaaaatatcattctaataaagtacatgcattaaacaatttt

SEQ ID NO: 22 - DNA, 1098 bp, Homo sapiens
atgtcagccccactggatgccgccctccacgcccttcaggaggagcaggccagaccgccctccacgcccttcaggaggagcaggccagactcaagatgaggctgtgggacctgcagcagctgagaaaggagctcggggactcccccaaagacaaggtcccattttcagtgcccaagatccccctggtattccgaggacacacccagcaggacccggaagtgcctaagtctttagtttccaatttgcggatccactgccctctgcttgcgggctctgctctgatcacctttgatgaccccaaagtggctgagcaggtgctgcaacaaaaggagcacacgatcaacatggaggagtgccggctgcgggtgcaggtccagcccttggagctgcccatggtcaccaccatccaggtgatggtgtccagccagttgagtggccggagggtgttggtcactggatttcctgccagcctcaggctgagtgaggaggagctgctggacaagctagagatcttctttggcaagactaggaacggaggtggcgatgtggacgttcgggagctactgccagggagtgtcatgctggggtttgctagggatggagtggctcagcgtctgtgccaaatcggccagttcacagtgccactgggtgggcagcaagtccctctgagagtctctccgtatgtgaatggggagatccagaaggctgagatcaggtcgcagccagttccccgctcggtactggtgctcaacattcctgatatcttggatggcccggagctgcatgacgtcctggagatccacttccagaagcccacccgcgggggcggggaggtagaggccctgacagtcgtaccccaaggacagcagggcctagcagtcttcacctctgagtcaggctaggggcctccccttctcatcctccccacccccccgccaaggttctcacactggcctgggcttgggtgcccatataggaggtctgtatgttcaccaacagtgcggaggggtcacacattgcaaaacactgcccagaacagtaaaaagagcctgcatgccaaaaaaaaaaaaaaaaaaaaaaaaaaaaaaaaaaaaaaaa

SEQ ID NO: 23 - DNA, 2359 bp, Homo sapiens
gttttgcctgctagcatctccctgtaactctcccaatcttgaggagtgatccctgtcccagcccctggaaaggggcaggaacgacaaactcaaagtccaggatgttcaccatgacaagagccatggaagaggctctttttcagcacttcatgcaccagaagctggggatcgcctatgccatacacaagccatttcccttctttgaaggcctcctagacaactccatcatcactaagagaatgtacatggaatctctggaagcctgtagaaatttgatccctgtatccagagtggtgcacaacattctcacccaactggagaggacttttaacctgtctcttctggtgacattgttcagtcaaattaacctgcgtgaatatcccaatctggtgacgatttacagaagcttcaaacgtgttggtgcttcctatgaacggcagagcagagacacaccaatcctacttgaagccccaactggcctagcagaaggaagctccctccataccccactggcgctgcccccaccacaaccccctcaaccaagctgttcaccctgtgcgccaagagtcagtgagcctggaacatcctcccagcaaagcgatgagatcctgagtgagtcgcccagcccatctgaccctgtcctgcctctccctgcactcatccaggaaggaagaagcacttcagtgaccaatgacaagttaacatccaaaatgaatgcggaagaagactcagaagagatgcccagcctcctcactagcactgtgcaagtggccagtgacaacctgatcccccaaataagagataaagaagaccctcaagagatgccccactctcccttgggctctatgccagagataagagataattctccagaaccaaatgacccagaagagccccaggaggtgtccagcacaccttcagacaagaaaggaaagaaaagaaaaagatgtatctggtcaactccaaaaaggagacataagaaaaaaagcctcccaagagggacagcctcatctagacacggaatccaaaagaagctcaaaagggtggatcaggttcctcaaaagaaagatgactcaacttgtaactccacggtagagacaagggcccaaaaggcgagaactgaatgtgcccgaaagtcgagatcagaggagatcattgatggcacttcagaaatgaatgaaggaaagaggtcccagaagacgcctagtacaccacgaagggtcacacaaggggcagcctcacctgggcatggcatccaagagaagctccaagtggtggataaggtgactcaaaggaaagacgactcaacctggaactcagaggtcatgatgagggtccaaaaggcaagaactaaatgtgcccgaaagtccagatcgaaagaaaagaaaaaggagaaagatatctgttcaagctcaaaaaggagatttcagaaaaatattcaccgaagaggaaaacccaaaagtgacactgtggattttcactgttctaagctccccgtgacctgtggtgaggcgaaagggattttatataagaagaaaatgaaacacggatcctcagtgaagtgcattcggaatgaggatggaacttggttaacaccaaatgaatttgaagtcgaaggaaaaggaaggaacgcaaagaactggaaacggaatatacgttgtgaaggaatgaccctaggagagctgctgaagcggaaaaactcggatgaatgcgaggtgtgctgtcaagggggacaacttctctgctgcggtacttgtccacgagtcttccatgaggactgtcacatcccccctgtggaagccaagaggatgctgtggagttgcaccttctgcaggatgaagaggtcttcaggaagccaacagtgccatcatgtatctaagaccctggagaggcagatgcagcctcaggaccagctgattcgagattacggtgagccctttcaggaagcaatgtggttggacctggttaaggaaaggctgattacggaaatgtacacggtggcatggtttgtgcgagacatgcgcctgatgtttcgcaaccataaaacattttacaaggcttctgactttggccaggtaggacttgacttagaggcagaatttgaaaaagatctcaaagacgtgctcggttttcatgaagccaatgacggcggtttctggactcttccttgaccctgttctgtaaagactgaagcatccccacctcaggattcagctgatgggaccctggcttggactgttgattgccagtgagtctgggatgtaattggctgccctcaggacccaaacccagacacttcataggattatcacaccctccatctttattctttctttttacctttaaaagtctatatcta

SEQ ID NO: 24 - DNA, 3200 bp, Homo sapiens
caggaagggccatgaagattaataaagatttggactcagggcaaatatttacttagtagcaataactcaaagaattactgttgaataaataagccaattaagcagccaatcacgtactatgcggatgcacacaaatgaaaccctcacttcaacctgaagacattcgcacatgagttacgtagagggacctgcaggaagcggtagagaaaacataaggcttatgcgtttaatttccacaccaatttcaggatctttgtcactgacagcagcactaagacttgttaactttatatagttaagaagaacaaggctgagcgcgatgactcacgcctgtaagcctagaactttgggaggccaaagcaggcagactgcttgagcccaggagttccagaccagcctgggcaacatggcaacaccccatctctacaaaaaaatacaagaatcagctgggcgtggtgatgtgttcctgtaatctcagctactcgggaggcagaggcaggaggattgcttgaacccgggaggcagaggttgtagttagccgagatctcgccactgcactccagtctggacgacagagtgagactcagtctcaaataaataaataaatacataaatataaggaaaaaaataaagctgctttctcctcttcctcctctttggtctcatctggctctgctccaggcatctgccacaatgtgggtgcttacacctgctgcttttgctgggaagttcttgagtgtgttcaggcaacctctgagctctctgtggaggagcctggtcccgctgttctgctggctgagggcaaccttctggctgctagctaccaagaggagaaagcagcagctggtcctgagagggccagatgagaccaaagaggaggaagaggaccctcctctgcccaccaccccaaccagcgtcaactatcacttcactcgccagtgcaactacaaatgcggcttctgtttccacacagccaaaacatcctttgtgctgccccttgaggaagcaaagagaggattgcttttgcttaaggaagctggtatggagaagatcaacttttcaggtggagagccatttcttcaagaccggggagaatacctgggcaagttggtgaggttctgcaaagtagagttgcggctgcccagcgtgagcatcgtgagcaatggaagcctgatccgggagaggtggttccagaattatggtgagtatttggacattctcgctatctcctgtgacagctttgacgaggaagtcaatgtccttattggccgtggccaaggaaagaagaaccatgtggaaaaccttcaaaagctgaggaggtggtgtagggattatagaatccctttcaagataaattctgtcattaatcgtttcaacgtggaagaggacatgacggaacagatcaaagcactaaaccctgtccgctggaaagtgttccagtgcctcttaattgaaggtgagaattgtggagaagatgctctaagagaagcagaaagatttgttattggtgatgaagaatttgaaagattcttggagcgccacaaagaagtgtcctgcttggtgcctgaatctaaccagaagatgaaagactcctaccttattctggatgaatatatgcgctttctgaactgtagaaagggacggaaggacccttccaagtccatcctggatgttggtgtagaagaagctataaaattcagtggatttgatgaaaagatgtttctgaagcgaggaggaaaatacatatggagtaaggctgatctgaagctggattggtagagcggaaagtggaacgagacttcaacacaccagtgggaaaactcctagagtaactgccattgtctgcaatactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaagaaataagctctagtgatatctgtgggggcaatatttaatttggatttgattttttaaaacaatgtttactgcgatttctatatttccattttgaaactatttcttgttccaggtttgttcatttgacagagtcagtattttttgccaaatatccagataaccagttttcacatctgagacattacaaagtatctgcctcaattatttctgctggttataatgcttttttttttttttgcttttatgccattgcagtcttgtactttttactgtgatgtacagaaatagtcaacagatgtttccaagaacatatgatatgataatcctaccaattttcaagaagtctctagaaagagataacacatggaaagacggcgtggtgcagcccagcccacggtgcctgttccatgaatgctggctacctatgtgtgtggtacctgttgtgtccctttctcttcaaagatccctgagcaaaacaaagatacgctttccatttgatgatggagttgacatggaggcagtgcttgcattgctttgttcgcctatcatctggccacatgaggctgtcaagcaaaagaataggagtgtagttgagtagctggttggccctacatttctgagaagtgacgttacactgggttggcataagatatcctaaaatcacgctggaaccttgggcaaggaagaatgtgagcaagagtagagagagtgcctggatttcatgtcagtgaagccatgtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaataaaaggtcattttaatttaaaaaaaaaaaaaaaaaaaaaaaaaaaaaaaaggcca

SEQ ID NO: 25 - DNA, 656 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 26 - DNA, 4759 bp, Homo sapiens
tagttattaaagttcctatgcagctccgcctcgcgtccggcctcatttcctcggaaaatccctgctttccccgctcgccacgccctcctcctacccggctttaaagctagtgaggcacagcctgcggggaacgtagctagctgcaagcagaggccggcatgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatgatatcaggtcctcaatcttcagctacagggaatgagtaactttgagtggagaagaaacaaacatagtgggtataatcatggatcgcttgtacccctgtgaaaatatattttttaaaaatatctttagcagtttgtactatattatatatgcaaagcacaaatgagtgaatcacagcactgagtattttgtaggccaacagagctcatagtacttgggaaaaattaaaaagcctcatttctagccttctttttagagtcaactgccaacaaacacacagtaatcactctgtacacactgggatagatgaatgaatggaatgttgggaatttttatctccctttgtctccttaacctactgtaaactggcttttgcccttaacaatctactgaaattgttcttttgaaggttaccagtgactctggttgccaaatccactgggcacttcttaaccttctatttgacctctgcgcatttggccctgttgagcactcttcttgaagctctccctgggcttctctctcttctagttctattctagtctttttttattgagtcctcctctttgctgatcccttccaagggttcaatatatatacatgtatatactgtacatatgtatatgtaactaatatacatacatacaggtatgtatatgtaatggttatatgtactcatgttcctggtgtagcaacgtgtggtatggctacacagagaacatgagaacataaagccatttttatgcttactactaaaagctgtccactgtagagttgctgtatgtagcaatgtgtatccactctacagtggtcagcttttagtagagagcataaaaatgataaaatacttcttgaaaacttagtttactatacatcttgccctattaatatgttctcttaacgtgtgccattgttctctttgaccattttcctataatgatgttgatgttcaacacctggactgaatgtctgttctcagatcccttggatgttacagatgaggcagtctgactgtcctttctacttgaaagattagaatatgtatccaaatggcattcacgtgtcacttagcaaggtttgctgatgcttcaaagagcttagtttgcggtttcctggacgtggaaacaagtatctgagttccctggagatcaacgggatgaggtgttacagctgcctccctcttcatgcaatctggtgagcagtggtgcaggcggggagccagagaaacttgccagttatataacttctctttggcttttcttcatctgtaaaacaaggataatactgaactgtaagggttagtggagagtttttaattaaaagaatgtgtgaaaagtacatgacacagtagttgcttgataatagttactagtagtagtattcttactaagacccaatacaaatggattatttaaaccaagtttatgagttggttttttttcattttctatttgtattttattaagagtgtcttttcttatgtgattttttttaattgctatttgatatggtttggctatatgtccccacccaaatctcatcttgaattataatccccatgtgtcaagggagggacctgacgggaggtgattggatcacgggggcagttgtccccatgctgttcttgggatagtgagttagttctcatgagatctgatggttttataagtgtttgacaattcctcctttacacacactctctctctcatctgctgccatgtaagacttgcctgcttccccttctgccatgattgtaagtttcctgaggcctcctcagccatgtggaactgtgaatctattaagcctcttttctttataaatgaaaa

SEQ ID NO: 27 - DNA, 1714 bp, Homo sapiens
ggggcattttgtgcctgcctagctatccagacagagcagctaccctcagctctagctgatactacagacagtacaacagatcaagaagtatggcagtgacaactcgtttgacacggttgcacgaaaagatcctgcaaaatcattttggagggaagcggcttagccttctctataagggtagtgtccatggattccgtaatggagttttgcttgacagatgttgtaatcaagggcctactctaacagtgatttatagtgaagatcatattattggagcatatgcggaagagagttaccaggaaggaaagtatgcttccatcatcctttttgcacttcaagatactaaaatttcagaatggaaactaggactatgtacaccagaaacactgttttgttgtgatgttacaaaatataactccccaactaatttccagatagatggaagaaatagaaaagtgattatggacttaaagacaatggaaaatcttggacttgctcaaaattgtactatctctattcaggattatgaagtttttcgatgcgaagattcactggatgaaagaaagataaaaggggtcattgagctcaggaagagcttactgtctgccttgagaacttatgaaccatatggatccctggttcaacaaatacgaattctgctgctgggtccaattggagctgggaagtccagctttttcaactcagtgaggtctgttttccaagggcatgtaacgcatcaggctttggtgggcactaatacaactgggatatctgagaagtataggacatactctattagagacgggaaagatggcaaatacctgccgtttattctgtgtgactcactggggctgagtgagaaagaaggcggcctgtgcagggatgacatattctatatcttgaacggtaacattcgtgatagataccagtttaatcccatggaatcaatcaaattaaatcatcatgactacattgattccccatcgctgaaggacagaattcattgtgtggcatttgtatttgatgccagctctattcaatacttctcctctcagatgatagtaaagatcaaaagaattcgaagggagttggtaaacgctggtgtggtacatgtggctttgctcactcatgtggatagcatggatttgattacaaaaggtgaccttatagaaatagagagatgtgagcctgtgaggtccaagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaatagggaatctaagggaggaaattatcaactgtgcacaaggaaaaaaatagatatgtgaaaggttcacgtaaatttcctcacatcacagaagattaaaattcagaaaggagaaaacacagaccaaagagaagtatctaagaccaaagggatgtgttttattaatgtctaggatgaagaaatgcatagaacattgtagtacttgtaaataactagaaataacatgatttagtcataattgtgaaaaatagtaataatttttcttggatttatgttctgtatctgtgaaaaaataaatttcttataaaactcggaaaaaaaaaaaaaaaaaaaaaaaaaaaaa

SEQ ID NO: 28 - DNA, 2645 bp, Homo sapiens
gcaagttcctgagagccgggaagaactgtaggaatagtcacagcttgacaaccgaacacaacctgagtgtgctgagaactcatggcgttgaccacctgagctataatgagctatgccaactcttgtttcagaacgacccctggcttttgccagaaatttgccaacattacaacaaaggagatggaccccacggctcttgtgcctttcaaaagcagtgcatcaagctccatatctgccagtattttttacagggggaatgcaagtttggcactagctgtaagagatcccatgatttctctaattctgagaatctggaaaaattggagaagttgggtatgagctcagacctggtgagcaggctgcctaccatttatagaaatgcacatgacatcaagaataagagctctgcccccagcagagtgcctcctctttttgtcccacaggggacttctgaaagaaaagacagttcaggttctgtgtccccaaacactcttagccaggaggagggtgatcagatctgtttgtaccatatccggaaaagttgtagctttcaagataagtgccatagagttcatttccatttgccgtatcgatggcaattcttggatagaggcaaatgggaggatttggacaacatggaacttattgaagaggcatattgcaatcccaaaatagaaaggatcctgtgctctgagtcagccagtacctttcactctcattgtctgaactttaacgccatgacttacggtgctacccaggctcgccgcctctccacggcctcctctgtcaccaaacctccacacttcatcctcaccactgactggatttggtactggagtgatgagtttggttcttggcaggaatatggaagacagggcacggtgcaccctgtgaccactgtcagcagtagcgacgtggagaaggcctacctggcctactgtacaccggggtctgacggccaggcagccaccttgaagttccaggccggaaagcacaactacgagttagatttcaaagccttcgttcagaaaaacctggtctatggcacaactaaaaaggtttgccgcagacccaaatacgtgtctccccaggatgtgacgaccatgcaaacctgcaataccaagtttccaggcccgaagagcatcccagactattgggactcctctgccctgccagacccaggctttcagaagatcacccttagttcttcctcggaagagtatcagaaggtctggaacctctttaaccgcacgctgcctttctactttgttcagaagattgagcgagtacagaacctggccctctgggaagtctaccagtggcaaaaaggacagatgcagaagcagaacggagggaaggccgtggacgagcggcagctgttccacggcaccagcgccatttttgtggacgccatctgccagcagaactttgactggcgggtctgtggtgttcatggcacttcctacggcaaggggagctactttgcccgagatgctgcatattcccaccactacagcaaatccgacacgcagacccacacgatgttcctggcccgggtgctggtgggcgagttcgtcaggggcaatgcctcctttgtccgtccgccggccaaggagggctggagcaacgccttctatgatagctgcgtgaacagtgtgtccgacccctccatctttgtgatctttgagaaacaccaggtctacccagagtatgtcatccagtacaccacctcctccaagccctcggtcacaccctccatcctgctggccttgggctccctgttcagcagccgacagtgagcgcacaggagtgttccaggcctttcacctgctctgccttgaaatggctatttgggcctttccttttctttttaaacagaaacttttaatgaactgttctcttaacattgacctctcaatgaagttatgttcttaatctcttgctaataatgatttttacttttaagtcacttttgggttcactagtggattaaccagaagtgattgtagttgagtccagttttgctttttaataatgtgttgaagttttagtttttactctttgttgactttgctgcttattggcaccagggacagagtttctagatacaattttatggattggttttaatttttatgagtttgtctctgcagtgattcggtttctcagagtctcatggcatcatagtttttccagaatgacacagtagccaccggtggatgacagcccacgggcggcacagtcacttctgcctgttgctctgacaccaacccaggcagctctgctgtggcttctcctgggctctggcattagttggtctgtgtcacattgtcagaacaggtggctgctgtgtggtgccatcgagtccctgctggttccccttgtcctgggagggtcacccattgcccaaggaagtgcatccacctggcaggtgacctggaggagtagcttccccgaggacccccaggcttggcctgtgattgcgcaaacccacatttcctaagcacactggacacccttcgagtgtgggttttaacatccctgtgagattgaatacttgtgccacacatgtcacaaaagagtatggaaataaaagaaaatttatccgaaaaaaaaaaaaaaaaaaaaaaaaaaaaaaaaaaaaa

SEQ ID NO: 29 - DNA, 2058 bp, Homo sapiens
gcacgaggaagccacagatctcttaagaactttctgtctccaaaccgtggctgctcgataaatcagacagaacagttaatcctcaatttaagcctgatctaacccctagaaacagatatagaacaatggaagtgacaacaagattgacatggaatgatgaaaatcatctgcgcaactgcttggaaatgtttctttgagtcttctctataagtctagtgttcatggaggtagcattgaagatatggttgaaagatgcagccgtcagggatgtactataacaatggcttacattgattacaatatgattgtagcctttatgcttggaaattatattaatttacgtgaaagttctacagagccaaatgattccctatggttttcacttcaaaagaaaaatgacaccactgaaatagaaactttactcttaaatacagcaccaaaaattattgatgagcaactggtgtgtcgtttatcgaaaacggatattttcattatatgtcgagataataaaatttatctagataaaatgataacaagaaacttgaaactaaggttttatggccaccgtcagtatttggaatgtgaagtttttcgagttgaaggaattaaggataacctagacgacataaagaggataattaaagccagagagcacagaaataggcttctagcagacatcagagactataggccctatgcagacttggtttcagaaattcgtattcttttggtgggtccagttgggtctggaaagtccagttttttcaattcagtcaagtctatttttcatggccatgtgactggccaagccgtagtggggtctgataccaccagcataaccgagcggtataggatatattctgttaaagatggaaaaaatggaaaatctctgccatttatgttgtgtgacactatggggctagatggggcagaaggagcaggactgtgcatggatgacattccccacatcttaaaaggttgtatgccagacagatatcagtttaattcccgtaaaccaattacacctgagcattctacttttatcacctctccatctctgaaggacaggattcactgtgtggcttatgtcttagacatcaactctattgacaatctctactctaaaatgttggcaaaagtgaagcaagttcacaaagaagtattaaactgtggtatagcatatgtggccttgcttactaaagtggatgattgcagtgaggttcttcaagacaactttttaaacatgagtagatctatgacttctcaaagccgggtcatgaatgtccataaaatgctaggcattcctatttccaatattttgatggttggaaattatgcttcagatttggaactggaccccatgaaggatattctcatcctctctgcactgaggcagatgctgcgggctgcagatgattttttagaagatttgcctcttgaggaaactggtgcaattgagagagcgttacagccctgcatttgagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagcacagtgcctggtaatgagcaagcatacttgccattacttttccttcccactctctccaacatcacattcactttaaatttttctgtatatagaaaggaaaactagcctgggcaacatgatgaaaccccatctccactgc

SEQ ID NO: 30 - DNA, 860 bp, Homo sapiens
ggatggcaaccttcagctagactgcctggctcaagggtggaagcaataccaacagagagcatttggctggttccggtgttcctcctgccagcgaagttgggcttccgccaagtgcagattctgtgccacacgtactgggagcactggacatcccagggtcaggtgcgtatgaggctctttggccaaaggtgccagaagtgctcctggtcccaatatgagatgcctgagttctcctcggatagcaccatgaggattctgagcaacctggtgcagcatatactgaagaaatactatggaaatggcatgaggaagtctccagaaatgccagtaatcctggaagtgtccctggaaggatcccatgacacagccaattgtgaggcatgcactttgggcatatgtggacagggcttaaaaagctacatgacaaagccgtccaaatccctactcccccacctaaagactgggaattcctcacctggaattggtgctgtgtacctcgcaaaccaagccaagaaccagtcagatgaggcaaaagaggctaaggggagtgggtatgagaaattagggcccagtcgagacccagatccactgaacatctgtgtctttattttgctgcttgtatttattgtagtcaaatgctttacatcagaatgatgaaaataggcttgccactttctcttattttaattccatggtagtcaatgaactggctgccactttaatataactgaaaattcattttgagaccaagcaggatcaagtttgtagaataaacactggtttcctagccatcctctgaaaacagtatgaaacatgaccaagtacataatggatttagtaataaatattgtcgaattgct

SEQ ID NO: 31 - DNA, 449 bp, Homo sapiens
caggccaaaaagtggtcccgcgtgcccttctccgtgcctgactttgacttcctgcagcattgtgccgagaacttgtcggacctctccctggactgaccacctcattgctgcagtgcccggtttgggctgtagggggcgggagagtctgcagcagactccaggcccctccttcctgaatcatcagctgtgggcatcaggcccaccagccacacaggagtcctgggcaccctggcttaggctcccgcaatgggaaaacaaccggagggccagagcttagtccagacctaccttgtacgcacatagacattttcatatgcactggatggagttagggaaactgaggcaaaagaatttgccatactgtactcagaatcacgacattccttccctaccaaggccacttctattttttgaggctcctcataaaaataaatgaaaaaatgggatag

SEQ ID NO: 32 - DNA, 3638 bp, Homo sapiens
ggtagatgcggctgtgacagcagcaaagaatgacggccaagggcgacagcaggggctggccatgctgtaaaggggcttcttgggagggtccagcctcaggaatcaaggggaactcctgagccgagaattctgaagatctcctccctccctgaagctgtgggctgggccatcggaaaactttcagttttgtttccttgcctgcaagaaacgaaactcaaccgaaagcctgcagagagcagaacatggaaggagacttctcggtgtgcaggaactgtaaaagacatgtagtctctgccaacttcaccctccatgaggcttactgcctgcggttcctggtcctgtgtccggagtgtgaggagcctgtccccaaggaaaccatggaggagcactgcaagcttgagcaccagcaggttgggtgtacgatgtgtcagcagagcatgcagaagtcctcgctggagtttcataaggccaatgagtgccaggagcgccctgttgagtgtaagttctgcaaactggacatgcagctcagcaagctggagctccacgagtcctactgtggcagccggacagagctctgccaaggctgtggccagttcatcatgcaccgcatgctcgcccagcacagagatgtctgtcgcagtgaacaggcccagctcgggaaaggggaaagaatttcagctcctgaaagggaaatctactgtcattattgcaaccaaatgattccagaaaataagtatttccaccatatgggtaaatgttgtccagactcagagtttaagaaacactttcctgttggaaatccagaaattcttccttcatctcttccaagtcaagctgctgaaaatcaaacttccacgatggagaaagatgttcgtccaaagacaagaagtataaacagatttcctcttcattctgaaagttcatcaaagaaagcaccaagaagcaaaaacaaaaccttggatccacttttgatgtcagagcccaagcccaggaccagctcccctagaggagataaagcagcctatgacattctgaggagatgttctcagtgtggcatcctgcttcccctgccgatcctaaatcaacatcaggagaaatgccggtggttagcttcatcaaaaggaaaacaagtgagaaatttcagctagatttggaaaaggaaaggtactacaaattcaaaagatttcacttttaacactggcattcctgcctacttgctgtggtggtcttgtgaaaggtgatgggttttattcgttgggctttaaaagaaaaggtttggcagaactaaaaacaaaactcacgtatcatctcaatagatacagaaaaggcttttgataaaattcaacttgacttcatgttaaaaaccctcaacaaaccaggcgtcgaaggaacatacctcaaaataataagagccatctatgacaaaaccacagccaacatcatactgaatgagcaaaagctggagcattactcttgagaagtagaacaaggcacttcagtcctattcaacatagtactggaagtcctcgccacagcaatcaggcaagagaaagaaataaaaggcaaccaaaaagaaaggaagtcgaagtatctctgtttgcagacgatatgattctatatctagaaaaccccatgatcttggcccaaaagctcctagatctgataaacaacttcagctaactttcaggagacaaaatcaatatacaaaatatggtagcatttttatacaccaacgacatccaagctgagagccaaatcaagaatgcaatcctattcacaattgccacaaaaagaataaaatacctaggaatacagctaaccagggagatgaaagatctctacaacaaaaattacaaaacactgctgaaagaaatcagagatgacacaaatggaaaaacattccatacttatggataggaagaatcaatattgttaaaatggccatactacccaaagcaatttatagattcaatgctattcctatcaaactaccaataacattcttcacagaatcagaaaaaaaaagcattaaaatttatttgaaaccaaaaaagagcccaaaaagccaaagcaatcctaagcaaaaagaacaaagctggaggcatcgcattacccaacttcaaactatactacagggctacagtaaccaaaactgcatgatactggtacaaaagcatggtgctggtacaaaagcagacacatagatcaatggaacagaatagagggcccagaaataaagctacacacctacaaccatctaatctttgacaaagttgacaaaaatacgcaatggggaaagaattccccattcagtaagtggtactgggataactagctagccatatgcagaggattgaaactgaaccacttccttacaccatatgcaaaaatcaactcaagatggattaaagacttaaatgtaaaaccccaaactataaaaactctggaagataacctaggcaataccattctggacataggaacggaaaaagatttcatgacaaagatcccaaaaataattgtaacgaaagcaaaaattgacaaatgggacatgattaaacagaattaccatttgactcagcaatcccattattggttatatacccaaaggaatctaaatcattctgtcataaagacatatatacacaaatgttcacggcagcactatacacaatcgcaaagtcagggaatcaaactaaatgtccatcagtggtagaaaggataaagaaaatgtggtggcagggagtggtggctcatgtctgtaatcccagcactttgggaggctgaggcgggtggttcacctgaggtcaggagtttgagaccagcctggccaacatggcgaaactccgtctccgctaaaaatacgaaaattagccaggcgtggtggcgagcacctgtcatcccagctacttgggaggcctaggcgtgagaatcgcttgaacctggaaggtggtggttgcagtgagccgagatcctgccactgcactccagcctgggcaaccaagcgagactctgccttaaaaaaaaaaaaaagaaaatgtggcacatatacaccatggaatactatgcagccataaaaaagaatgggatcatgtcctgtgcagcaacgtggatggagctggaagccattatcctaaatgaactcactcagaaacagaaaaccaaataccacatgttctcacttataagtagaagctaaacattgagtacacatggatacaaagaagggaaccgcagacactggggcctacctgaggtcggagcatggaaggagggtgaggatcaaaaaactacctatctggtactatgctttttatctggatgatgaaataatctgtacaacaaaccctggtgacatgcaatttacctatatagcaagcctacacatgtgcccctgaacctaaaaaaaaagttaaaagaaaaacgtttggattattttccctctttcgaacaaagacattggtttgcccaaggactacaaataaaccaacgggaaaaaagaaaggttccagttttgtctgaaaattctgattaagcctctgggccctacagcctggagaacctggagaatcctacacccacagaacccggctttgtccccaaagaataaaaacacctctctaaaaaaaaaaaaaaaa

SEQ ID NO: 33 - DNA, 1673 bp, Homo sapiens
tcccttctgaggaaacgaaaccaacagcagtccaagctcagtcagcagaagagataaaagcaaacaggtctgggaggcagttctgttgccactctctctcctgtcaatgatggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctggctgaaagcaacagtgcagacgatgagaccgacgatcccaggaggtatcagaaatatggttacattggaacacatgagtaccctcatttctctcatagacccagcacactccaggcagcatccaccccacaggcagaagaggactggacctgcaccatcctctgaatgccagtgcatcttgggggaaagggctccagtgttatctggaccagttccttcattttcaggtgggactcttgatccagagaggacaaagctcctcagtgagctggtgtataatccaggacagaacccaggtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccatttaaaaaaaaaaa

SEQ ID NO: 34 - DNA, 6031 bp, Homo sapiens
gctgggtcctaggccaggtctggggtaacctggaacttccacctgggctctgcgctaggtctctgtttcactccctccccgcggggcgcgcagctcgcgggtctttggacaccaccggtcctgagtccgcggactgccattttcattaagaactgccacttagaggtaccaaaataaagggtatttgctacctttaatacttgccagttcaggttggaggcacaggcagcagcaagaatggaaagaaatgttcttacaacattttcacaggaaatgtcccagttaattttgaatgaaatgccaaaagctgaatattccagtttattcaatgattttgttgaatctgaattttttttgattgatggggattcattacttatcacatgtatctgtgagatatcatttaagcctgggcagaacctccatttcttctatctggttgaacgctatcttgtggatcttattagcaaaggaggacaattcaccatagttttcttcaaggatgccgagtatgcgtatttcaacttccctgaacttctttctttgagaactgctttaattcttcatcttcagaagaataccaccattgatgttcgaacaacattttcgagatgcttatcaaaagagtggggaagtttcttggaagagagttacccatatttcctgatagttgcagacgaaggcctgaacgatctacaaacacagcttttcaactttttaatcattcattcttgggcaaggaaggtcaacgttgtactttcctcagggcaagaatctgatgttctttgcctttatgcataccttcttccaagcatgtacagacaccagattttttcctggaagaataagcagaacattaaagatgcttatacaaccctgcttaaccagttggaaagatttaagctttcagcattagcacctctttttggaagtttaaaatggaataatattacggaagaggcacacaagactgtatctctgcttacacaagtctggccagaaggatctgacattcggcgtgtcttttgtgttacttcatgctcattatctttgagaatgtaccatcgctttttaggaaacagagagccctcctctggtcaggaaactgagatccaacaggtgaacagtaattgcttaaccctgcaggagatggaagatttgtgtaaactgcattgtctcactgtggtttttctactccatctgcctctttctcaaagagcttgtgctagagtcatcacttcacattgggctgaggacatgaagcctttattacaaatgaaaaagtggtgtgaatatttcatcttaagaaatatacatacttttgaattttggaatctgaatttaattcacctttctgacttaaatgatgagcttttgttgaagaatattgctttttactatgaaaatgaaaatgtaaaaggcctacatttgaatttgggagataccattatgaaagattatgaatatctctggaataccgtatcaaagttggtcagagactttgaggttggacagccatttcctctgagaacaacaaaagtttgttttcttggaaagaaaccatcaccaatcaaagacagctccaatgaaatggtgcccaatttgggttttattccaacgtcatcttttgtggttgataaatttgctggagatattttgaaagatttgccttttctaaagagtgatgatcctattgttacttcactggttaaacaaaaggaatttgatgaacttgtgcactggcattctcataaacccctgagtgatgattatgacaggtccaggtgtcagtttgatgaaaaatctagagaccctcgtgttcttagatctgtgcaaaagtatcatgttttccaacggttttatgggaattcattagaaacagtctcttcgaaaatcatcgtgactcaaactattaagtcaaagaaggattttagtgggcccaagagcaaaaaggcacacgagaccaaggctgaaataattgctagagagaataagaaaaggttatttgccagggaagaacaaaaggaagagcaaaagtggaatgctttgtcattttctattgaagagcaattgaaagaaaatttacactctggaataaagagcctggaagattttttgaaatcctgtaaaagtagctgtgtgaaacttcaggttgaaatggtggggttaactgcttgcttgaaagcctggaaagaacattgccgaagtgaagaaggtaaaaccacgaaagatttaagtatagctgttcaggtgatgaaaaggatccactccttgatggaaaaatactcagaacttttacaagaagatgatcggcaactcatagccagatgccttaagtatttaggatttgatgagttggcaagttctttacatccagcccaggatgcagaaaatgatgtaaaagtgaagaaaaggaataaatattcaattggcattgggccagctcggttccaactgcaatacatgggccattatttgatacgagatgagagaaaagacccagatcccagggtccaggattttattcccgacacatggcagcgagagctccttgatgttgtggataagaatgagtcagcagtgattgttgccccaacgtcctcaggcaaaacatatgcctcctactactgtatggagaaagtgctgaaggagagcgacgacggggtggtcgtgtacgttgcacccacaaaggcccttgttaatcaagtggcagcaactgttcagaatcgttttacgaaaaatctgccaagtggtgaagttctctgtggtgttttcaccagggagtatcgtcatgatgccttaaactgtcaggtacttattacagtgcctgcctgctttgaaattctgctgcttgctcctcatcgccaaaactgggtgaaaaagatcagatatgttatatttgatgaggttcattgtcttggtggagaaattggagcagaaatctgggaacatctccttgtcatgatccgatgtccctttttggctctttcagctaccataagtaatcctgaacatctcaccgagtggctacaatcggtaaaatggtactggaaacaagaagacaaaataattgaaaataataccgcttctaaaagacatgtgggtcgtcaggccggctttcccaaagactacttgcaagtaaaacaatcgtataaagttagacttgtgctctatggagagaggtataatgatctagagaagcatgtatgttcaataaaacatggtgacattcattttgatcattttcacccatgtgctgcactaacaacagatcatattgaaaggtatggattccctcctgatcttaccctttcacctcgagaaagcatccagctgtatgatgccatgtttcaaatttggaaaagttggcctcgggcccaggaactgtgcccagaaaacttcattcattttaacaataaattagtcattaaaaagatggatgctaggaaatatgaagagagtctaaaggcagaattaacaagttggattaaaaatggcaacgtagagcaggccagaatggtacttcagaatcttagtcctgaagcagatttgagtccagaaaacatgatcaccatgtttccacttctagttgaaaaactaaggaaaatggagaagttacctgcactattttttttattcaagttaggagctgtagaaaacgcagctgaaagtgtgagcactttcctaaagaaaaagcaggagacaaaaaggcctcccaaagctgataaagaagcccatgtcatggctaacaaacttcgaaaagttaaaaaatccatagagaaacaaaagatcatagatgaaaagagccagaaaaaaaccagaaatgtggatcaaagcctaatacatgaagctgaacatgataatctagtgaagtgtctagagaagaacctggaaatcccacaggactgcacatatgctgatcaaaaagcagtggacactgagactttgcagaaggtatttggtcgagtaaaatttgaaagaaaaggtgaagaattgaaagccttggcagaaaggggtattggatatcatcacagtgctatgagtttcaaagaaaaacaattagttgaaatcctctttagaaaaggatatcttagggtggtgacagctactggaacacttgctttaggtgtcaacatgccttgtaaatctgtggtttttgctcaaaactcagtctatctggatgcgttgaattatagacagatgtctggccgtgctggaagaagaggtcaagacctgatgggagatgtatatttctttgatattccattccccaaaataggaaaactcataaaatccaatgttcctgagctgagaggacacttccctctcagcataaccctggtcctgcgactcatgctgctggcttccaagggagatgacccagaggataccaaggcaaaggtgctatcagtgctaaagcattcattgctgtccttcaagcaacccagagtcatggacatgttaaaactttacttcctgttttctttgcagttcctggtgaaagagggctatttagatcaagaaggtaatcctatggggtttgctggacttgtatcacatttgcattatcatgaaccttctaatcttgtttttgtcagttttcttgtaaatggactcttccatgatctctgtcagccaaccaggaaaggctcaaaacatttttctcaagacgttatggaaaagctagtattagtattggcacatctctttggaagaagatattttccaccaaagttccaagatgcacacttcgagttttatcaatcaaaggtgttccttgatgatctccctgaggattttagtgatgctttagatgaatataacatgaaaattatggaggactttaccactttcctacgaattgtttccaaactggctgatatgaatcaggaatatcaactcccattgtcaaaaatcaaattcacaggtaaagaatgtgaagactctcaactcgtatctcatttgatgagctgcaaggaaggaagagtagcaatttcaccatttgtttgtctgtctgggaactttgatgatgatttgcttcgactagaaactccaaaccatgttactctaggcacaatcggtgtcaatcgctctcaggctccagtgctgttgtcacagaaatttgataaccgaggaaggaaaatgtcgcttaatgcctatgcactggatttctacaaacatggttccttgataggattagtccaggataacaggatgaatgaaggagatgcttattatttgttgaaggattttgcactcaccattaaatctatcagtgtttccttgcgtgagctatgtgaaaatgaagacgacaacgttgtcttagcctttgaacaactgagtacaactttttgggaaaagttaaacaaagtctaaaaacaaagtctatgcaaaccacttaaaaataattccatagtagtttttcaggtcacgtttttgattcttatgcttcttgccagaaatacattatgataaagtggaaatacattacgatgaagtggaaagagcaaacactttggaatcaaacagagttgcaatcaaacctgcaatgttctgtcatgaatactcacaaattatttagtatacctgaatcttggtttctttttataactgagtaataatggttacatctcaggtagtttgaggattgactaaaaaaatgcgagaatgttgtatgtgactgaataacaatttttactctgcgaagccaaagtaaatataatattatcagtaactttatccccagtgtcagtatttataaaatgtttattaaggctagaaaaaatgaatacaatatcctgaaggtgaaatatattctcttcaattagcataaatatgatttacataagttagctatacagctattgagatagtactttctagtaaacttaaactactttttaaacatacattttgtgatgatttaacaaaaatatagagaatgatttgctttattgtaattgtatataagtgactggaaaagcacaaagaaataaagtgggttcgatctgttaaataaaaaaaaaaaaaa

SEQ ID NO: 35 - DNA, 632 bp, Homo sapiens
gccatctagtctgtggttttctgttgaagcagtctgaattgactaaaacagtcacttggagtagttataaaccactttcctgttgaaagcagaacatgctgattcaactgttttgttcaatagcaatgatagattttgtttaagtcccctacactttcttatttctaaatgatcaagagtacacttcctggcagtgattaaggagtgtgtatctaacagaaaaaatatatataccctgtgaacccgaatatggaattcagattgtttctgccctcagtatcatacttaaaaaacaagcatacaaacaaacataagggaacaaacagcaaccataacaaaaacaaacctttaaaggtgggtttttgctgtgataaatgaatacggtactctgaaggagaaaaaagtttctcaaatgagcttaaactgcaagtgatttaaaaattagagaatataattcttaaagctattgaaagtttcaaccagaaaacctcaagtgaattttgtatgtaaatgaaatcttgaatgtaagttctgtgattctttaagcaaacaattagctgaaaacttggtattgttgtagtttatgtagtaagtgacttggcacccatcagaaaataaagggcattaaattg

SEQ ID NO: 36 - DNA, 409 bp, Homo sapiens
aaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgtt

SEQ ID NO: 37 - DNA, 3903 bp, Homo sapiens
agcacttgaagttcaggcagcgagagttgacatggggccagggctgcgcccctggggcgggttgaagacagggtgagtctcttgatattcaggaaatcatcgcgcacccagtcaccagcgttcgggagcctgtcgcagcgggaccgacggaatccggagcaggcgacagggcgcagaagcgggatgtacttctgttggggcgccgactccagggagctgcagcgccggaggacggcgggcagccccggggctgagctactgcaggcggccagcggggagcgccactctctgctgctgctgaccaaccacagggtcctctcgtgcggagacaacagcaggggtcagctgggccgcaggggcgcgcagcgcggggagctgccagaaccaattcaggcattggaaaccctaattgttgatctcgtgagctgcgggaaggagcactccctggctgtgtgccacaaaggaagggtcttcgcatggggagctggttctgaagggcagctggggattggagaattcaaggaaataagtttcacacctaagaaaataatgactctgaatgatataaaaataatacaagtttcctgtggacactaccactccctggcattatcaaaagatagccaagtgttttcgtggggaaagaacagccatgggcagctgggcttggggaaggagttcccctcccaagccagcccgcagagggtgaggtccctggaggggatcccactggctcaggtggctgccggaggggctcacagctttgccctgtctctctgtgggacttcgtttggctggggaagtaacagtgccgggcagctggccctcagtgggcgtaatgtcccagtgcaaagcaacaagcctctctcagtcggtgcactgaagaatctaggtgtggtttatatcagctgtggtgatgcacacactgcggtgcttacccaggacgggaaagtgttcacatttggagacaatcgctctggacagctgggatacagccccactcctgagaagagaggtccacaacttgtggaaagaattgatggcctagtttcgcagatagattgtggaagttatcacaccctggcatatgtgcacaccactggtcaggtggtatcttttggtcatggaccaagtgacacaagcaagccaactcatccggaggccctgacagagaactttgacattagctgcctgatttctgctgaagacttcgtggatgttcaagtcaaacacatttttgctggaacatatgccaactttgtgacaactcatcaggatactagttccacacgtgctcccgggaaaaccctgccagaaataagccgaattagccagtccatggcagaaaaatggatagcagtgaaaagaagaagtactgaacatgaaatggctaaaagtgaaattagaatgatattttcatctcctgcttgtctgactgcaagttttttaaagaaaagaggaactggagaaacgacttccattgatgtggacttagaaatggcaagagataccttcaagaagttaacaaaaaaggaatggatttcttccatgataactacgtgtctcgaggatgatctgctcagagctcttccatgccattctccacaccaagaagctttatcagttttcctcctgctcccagaatgtcctgtgatgcatgattctaagaactggaagaacctggtggttccatttgcaaaggctgtgtgtgaaatgagtaaacaatctttgcaagtcctaaagaagtgttgggcatttttgcaagaatcttctctgaatccgctgatccagatgcttaaagcagccatcatctctcagctgcttcatcagactaaaaccgaacaggatcactgtaatgttaaagctcttttaggaatgatgaaagaactgcataaggtaaacaaagctaactgtcgactaccagaaaatactttcaacataaatgaactctccaacttattaaacttttatatagatagaggaagacagctctttcgggataaccacctgatacctgcagaaacccccagtcctgttattttcagtgattttccatttatctttaattcgctatccaaaattaaattattgcaagctgattcacatataaagatgcagatgtcagaaaagaaagcatacatgcttatgcatgaaacaattctgcaaaaaaaggatgaatttcctccatcacccagatttatacttagagtcagacgaagtcgcctggttaaagatgctctgcgtcaattaagtcaagctgaagctactgacttctgcaaagtattagtggttgaatttattaatgaaatttgtcctgagtctggaggggttagttcagagttcttccactgtatgtttgaagagatgaccaagccagaatatggaatgttcatgtatcctgaaatgggttcctgcatgtggtttcctgccaagcctaaacctgagaagaaaagatatttcctctttggaatgctgtgtggactctccttattcaatttaaatgttgctaaccttcctttcccactggctctgtataaaaaacttctggaccaaaagccatcattggaagatttaaaagaactcagtcctcggttggggaagagtttgcaagaagttctagatgatgctgctgatgacattggagatgcgctctgcatacgcttttctatacactgggaccaaaatgatgttgacttaattccaaatgggatctccatacctgtggaccaaaccaacaagagagactatgtttctaagtatattgattacattttcaacgtctctgtaaaagcagtttatgaggaatttcagagaggattttatagagtctgtgagaaggagatacttagacatttctaccctgaagaactaatgacagcaatcattggaaatactgattatgactggaaacagtttgaacagaattcaaagtatgagcaaggataccaaaaatcacatcctactatacagttgttttggaaggctttccacaaactaaccttggatgaaaagaaaaaattcctctttttccttacaggacgtgataggctgcatgcaagaggcatacagaaaatggaaatagtatttcgctgtcctgaaactttcagtgaaagagatcacccaacatcaataacttgtcataatattctctccctccctaagtattctacaatggaaagaatggaggaagcacttcaagtagccatcaacaacaacagaggatttgtctcacccatgctcacacagtcataatcacctctgagagactcagggtgggctttctcacacttggatccttctgttcttccttacacctaaataatacaagagattaatgaatagtggttagaagtagttgagggagagattgggggaatggggagatgatgatgatggtcaaagggtgcaaaatctcacacaagactgaggcaggagaatagggtacagagatagggatctaaggatgacttggacacactccctggcactgaagagtctgaacactggcctgtgattggtccattccaggaccttcatttgcataaggtatcaaaccacatcagcctctgattggccatgggccagacctgcactctggccaatgattggttcattccaggacattcatttgcataaggagtcaaaccacaccagtcttggattggctgtgagccaattcacctcagtctctaattggctgtgagtcagtctttcatttacatagggtgtaaccatcaagaaacctctacagggtacttaagccccagaagattttgctaccagggctcttgagccacttgctctagcccactcccaccctgtggaatgtactttcacttttgctgcttcactgccttgtgctccaataaatccactccttcaccacccaaaaaaaaaaaaaaa

SEQ ID NO: 38 - DNA, 1775 bp, Homo sapiens
gtaactgaaaatccacaagacagaatagccagatctcagaggagcctggctaagcaaaaccctgcagaacggctgcctaatttacagcaaccatgaggccacttaaggatgcagcaagaaggagccatctgcaatccaggaagaaattccttgccaggaaccaaattggttgtcaccttcatctaggacttctagcctcgagaacttacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaagaattatgaacgggccaaggcctgctttgaaaaggtgcttgaagtggaccctgaaaacccggaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaa

SEQ ID NO: 39 - DNA, 1977 bp, Homo sapiens
ggcagacaggaagacttctgaagaacaaatcagcctggtcaccagcttttcggaacagcagagacacagagggcagtcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatccgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggaggataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgccccctgcactccagcctgggcttcagagcaagactcgg